SEQUENCE LISTING (102 sequences)

SEQ ID NO: 1 - protein, 137 aa, Artificial Sequence
  RQR8
ACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 2 - protein, 431 aa, Artificial Sequence
  FOXP3, UniProtKB accession Q9BZS1
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGP

SEQ ID NO: 3 - protein, 431 aa, Artificial Sequence
  FOXP3, aa418 mutant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPSRCSNPTPGP

SEQ ID NO: 4 - protein, 431 aa, Artificial Sequence
  FOXP3, aa422 mutant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPARCSNPTPGP

SEQ ID NO: 5 - protein, 431 aa, Artificial Sequence
  FOXP3, aa 418 and 422 mutant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKREQRPARCSNPTPGP

SEQ ID NO: 6 - protein, 362 aa, Artificial Sequence
  FOXP3, truncated variant
GGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQQLVLEKEKLSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEF

SEQ ID NO: 7 - protein, 441 aa, Artificial Sequence
  FOXP3, illustrative variant
MPNPRPGKPSAPSLALGPSPGASPSWRAAPKASDLLGARGPGGTFQGRDLRGGAHASSSSLNPMPPSQLQLPTLPLVMVAPSGARLGPLPHLQALLQDRPHFMHQLSTVDAHARTPVLQVHPLESPAMISLTPPTTATGVFSLKARPGLPPGINVASLEWVSREPALLCTFPNPSAPRKDSTLSAVPQSSYPLLANGVCKWPGCEKVFEEPEDFLKHCQADHLLDEKGRAQCLLQREMVQSLEQVEELSAMQAHLAGKMALTKASSVASSDKGSCCIVAAGSQGPVVPAWSGPREAPDSLFAVRRHLWGSHGNSTFPEFLHNMDYFKFHNMRPPFTYATLIRWAILEAPEKQRTLNEIYHWFTRMFAFFRNHPATWKNAIRHNLSLHKCFVRVESEKGAVWTVDELEFRKKRSQRPSRCSNPTPGPEGRGSLLTCGDVEEN

SEQ ID NO: 8 - DNA, 1296 bp, Artificial Sequence
  FOXP3, Illustrative FOXP3 polynucleotide
atgcccaaccccaggcctggcaagccctcggccccttccttggcccttggcccatccccaggagcctcgcccagctggagggctgcacccaaagcctcagacctgctgggggcccggggcccagggggaaccttccagggccgagatcttcgaggcggggcccatgcctcctcttcttccttgaaccccatgccaccatcgcagctgcagctgcccacactgcccctagtcatggtggcaccctccggggcacggctgggccccttgccccacttacaggcactcctccaggacaggccacatttcatgcaccagctctcaacggtggatgcccacgcccggacccctgtgctgcaggtgcaccccctggagagcccagccatgatcagcctcacaccacccaccaccgccactggggtcttctccctcaaggcccggcctggcctcccacctgggatcaacgtggccagcctggaatgggtgtccagggagccggcactgctctgcaccttcccaaatcccagtgcacccaggaaggacagcaccctttcggctgtgccccagagctcctacccactgctggcaaatggtgtctgcaagtggcccggatgtgagaaggtcttcgaagagccagaggacttcctcaagcactgccaggcggaccatcttctggatgagaagggcagggcacaatgtctcctccagagagagatggtacagtctctggagcagcagctggtgctggagaaggagaagctgagtgccatgcaggcccacctggctgggaaaatggcactgaccaaggcttcatctgtggcatcatccgacaagggctcctgctgcatcgtagctgctggcagccaaggccctgtcgtcccagcctggtctggcccccgggaggcccctgacagcctgtttgctgtccggaggcacctgtggggtagccatggaaacagcacattcccagagttcctccacaacatggactacttcaagttccacaacatgcgaccccctttcacctacgccacgctcatccgctgggccatcctggaggctccagagaagcagcggacactcaatgagatctaccactggttcacacgcatgtttgccttcttcagaaaccatcctgccacctggaagaacgccatccgccacaacctgagtctgcacaagtgctttgtgcgggtggagagcgagaagggggctgtgtggaccgtggatgagctggagttccgcaagaaacggagccagaggcccagcaggtgttccaaccctacacctggcccctga

SEQ ID NO: 9 - DNA, 1352 bp, Artificial Sequence
  FOXP3, Illustrative FOXP3 polynucleotide
gaattcgtcgacatgcccaaccccagacccggcaagccttctgccccttctctggccctgggaccatctcctggcgcctccccatcttggagagccgcccctaaagccagcgatctgctgggagctagaggccctggcggcacattccagggcagagatctgagaggcggagcccacgcctctagcagcagcctgaatcccatgccccctagccagctgcagctgcctacactgcctctcgtgatggtggcccctagcggagctagactgggccctctgcctcatctgcaggctctgctgcaggaccggccccactttatgcaccagctgagcaccgtggacgcccacgccagaacacctgtgctgcaggtgcaccccctggaaagccctgccatgatcagcctgacccctccaaccacagccaccggcgtgttcagcctgaaggccagacctggactgccccctggcatcaatgtggccagcctggaatgggtgtcccgcgaacctgccctgctgtgcaccttccccaatcctagcgcccccagaaaggacagcacactgtctgccgtgccccagagcagctatcccctgctggctaacggcgtgtgcaagtggcctggctgcgagaaggtgttcgaggaacccgaggacttcctgaagcactgccaggccgaccatctgctggacgagaaaggcagagcccagtgcctgctgcagcgcgagatggtgcagtccctggaacagcagctggtgctggaaaaagaaaagctgagcgccatgcaggcccacctggccggaaagatggccctgacaaaagccagcagcgtggccagctccgacaagggcagctgttgtatcgtggccgctggcagccagggacctgtggtgcctgcttggagcggacctagagaggcccccgatagcctgtttgccgtgcggagacacctgtggggcagccacggcaactctaccttccccgagttcctgcacaacatggactacttcaagttccacaacatgaggccccccttcacctacgccaccctgatcagatgggccattctggaagcccccgagaagcagcggaccctgaacgagatctaccactggtttacccggatgttcgccttcttccggaaccaccccgccacctggaagaacgccatccggcacaatctgagcctgcacaagtgcttcgtgcgggtggaaagcgagaagggcgccgtgtggacagtggacgagctggaatttcggaagaagcggtcccagaggcccagccggtgtagcaatcctacacctggccctgagggcagaggaagtctgctaacatgcggtgacgtcgaggagaatcc

SEQ ID NO: 10 - protein, 157 aa, Artificial Sequence
  RQR8 with signal peptide
MGTSLLCWMALCLLGADHADACPYSNPSLCSGGGGSELPTQGTFSNVSTNVSPAKPTTTACPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 11 - protein, 5 aa, Artificial Sequence
  3PB2 VH CDR1
DYGMH

SEQ ID NO: 12 - protein, 17 aa, Artificial Sequence
  3PB2 VH CDR2
FIRNDGSDKYYADSVKG

SEQ ID NO: 13 - protein, 14 aa, Artificial Sequence
  3PB2 VH CDR3
NGESGPLDYWYLDL

SEQ ID NO: 14 - protein, 11 aa, Artificial Sequence
  3PB2 VL CDR1
QSSLDISHYLN

SEQ ID NO: 15 - protein, 7 aa, Artificial Sequence
  3PB2 VL CDR2
DASNLET

SEQ ID NO: 16 - protein, 9 aa, Artificial Sequence
  3PB2 VH CDR3
QQYDNLPLT

SEQ ID NO: 17 - protein, 117 aa, Artificial Sequence
  VH 3PB2
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGT

SEQ ID NO: 18 - protein, 107 aa, Artificial Sequence
  VL 3PB2
DVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 19 - protein, 249 aa, Artificial Sequence
  ScFv 3PB2
  VARIANT (118)..(142): Xaa can be any amino acid
  MISC_FEATURE (133)..(142): Any one or all of amino acids 133-142 can either be present or absent
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTXXXXXXXXXXXXXXXXXXXXXXXXXDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 20 - protein, 5 aa, Artificial Sequence
  3PF12 VH CDR1
DYGMH

SEQ ID NO: 21 - protein, 17 aa, Artificial Sequence
  3PF12 VH CDR2
FIRNDGSDKYYADSVKG

SEQ ID NO: 22 - protein, 14 aa, Artificial Sequence
  3PF12 VH CDR3
NGESGPLDYWYFDL

SEQ ID NO: 23 - protein, 11 aa, Artificial Sequence
  B11 VL CDR1
QASQDISNYLN

SEQ ID NO: 24 - protein, 7 aa, Artificial Sequence
  B11 VL CDR2
DASNLET

SEQ ID NO: 25 - protein, 9 aa, Artificial Sequence
  B11 VH CDR3
QQYDNLPPT

SEQ ID NO: 26 - protein, 117 aa, Artificial Sequence
  VH 3PF12
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGT

SEQ ID NO: 27 - protein, 108 aa, Artificial Sequence
  VL B11
DVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLG

SEQ ID NO: 28 - protein, 246 aa, Artificial Sequence
  ScFv
QVQLVQSGGGVVQPGGSLRVSCAASGVTLSDYGMHWVRQAPGKGLEWMAFIRNDGSDKYYADSVKGRFTISRDNSKKTVSLQMSSLRAEDTAVYYCAKNGESGPLDYWYFDLWGRGTLVTVSSGGGGSGGGGSGGGGSDVVMTQSPSSLSASVGDRVTITCQASQDISNYLNWYQQKPGKAPKLLIYDASNLETGVPSRFSGSGSGTDFTFTISSLQPEDIATYYCQQYDNLPPTFGGGTKLTVLG

SEQ ID NO: 29 - protein, 19 aa, Artificial Sequence
  P2A peptide - cleavage domain
ATNFSLLKQAGDVEENPGP

SEQ ID NO: 30 - protein, 18 aa, Artificial Sequence
  T2A  peptide - cleavage domain
EGRGSLLTCGDVEENPGP

SEQ ID NO: 31 - protein, 20 aa, Artificial Sequence
  E2A peptide - cleavage domain
QCTNYALLKLAGDVESNPGP

SEQ ID NO: 32 - protein, 22 aa, Artificial Sequence
  F2A peptide - cleavage domain
VKQTLNFDLLKLAGDVESNPGP

SEQ ID NO: 33 - protein, 17 aa, Artificial Sequence
  Rituximab binding epitope from CD20
CEPANPSEKNSPSTQYC

SEQ ID NO: 34 - protein, 10 aa, Artificial Sequence
  Rituximab mimotope
ACPYANPSLC

SEQ ID NO: 35 - protein, 10 aa, Artificial Sequence
  Rituximab mimotope
ACPYSNPSLC

SEQ ID NO: 36 - protein, 10 aa, Artificial Sequence
  Rituximab mimotope
ACPFANPSTC

SEQ ID NO: 37 - protein, 10 aa, Artificial Sequence
  Rituximab mimotope
ACNFSNPSLC

SEQ ID NO: 38 - protein, 10 aa, Artificial Sequence
  Rituximab mimotope
ACPFSNPSMC

SEQ ID NO: 39 - protein, 10 aa, Artificial Sequence
  Rituximab mimotope
ACSWANPSQC

SEQ ID NO: 40 - protein, 10 aa, Artificial Sequence
  Rituximab mimotope
ACMFSNPSLC

SEQ ID NO: 41 - protein, 10 aa, Artificial Sequence
  Rituximab mimotope
ACPFANPSMC

SEQ ID NO: 42 - protein, 10 aa, Artificial Sequence
  Rituximab mimotope
ACWASNPSLC

SEQ ID NO: 43 - protein, 10 aa, Artificial Sequence
  Rituximab mimotope
ACEHSNPSLC

SEQ ID NO: 44 - protein, 10 aa, Artificial Sequence
  Rituximab mimotope
ACWAANPSMC

SEQ ID NO: 45 - protein, 9 aa, Artificial Sequence
  Rituximab mimotope
CPYANPSLC

SEQ ID NO: 46 - protein, 9 aa, Artificial Sequence
  Rituximab mimotope
CPYSNPSLC

SEQ ID NO: 47 - protein, 9 aa, Artificial Sequence
  Rituximab mimotope
CPFANPSTC

SEQ ID NO: 48 - protein, 9 aa, Artificial Sequence
  Rituximab mimotope
CNFSNPSLC

SEQ ID NO: 49 - protein, 9 aa, Artificial Sequence
  Rituximab mimotope
CPFSNPSMC

SEQ ID NO: 50 - protein, 9 aa, Artificial Sequence
  Rituximab mimotope
CSWANPSQC

SEQ ID NO: 51 - protein, 9 aa, Artificial Sequence
  Rituximab mimotope
CMFSNPSLC

SEQ ID NO: 52 - protein, 9 aa, Artificial Sequence
  Rituximab mimotope
CPFANPSMC

SEQ ID NO: 53 - protein, 9 aa, Artificial Sequence
  Rituximab mimotope
CWASNPSLC

SEQ ID NO: 54 - protein, 9 aa, Artificial Sequence
  Rituximab mimotope
CEHSNPSLC

SEQ ID NO: 55 - protein, 9 aa, Artificial Sequence
  Rituximab mimotope
CWAANPSMC

SEQ ID NO: 56 - protein, 10 aa, Artificial Sequence
  Circular Rituximab mimotope consensus sequence
  VARIANT (1)..(1): Xaa can be Ala or absent
  VARIANT (3)..(3): Xaa can be any amino acid (More particularly, Xaa may be an amino acid selected from  Pro, Asn, Ser, Met, Trp or Glu)
  VARIANT (4)..(4): Xaa can be any amino acid (More particularly, Xaa may be an amino acid selected from Tyr, Phe, Trp, Ala or His)
  VARIANT (5)..(5): Xaa can be Ala or Ser
  VARIANT (9)..(9): Xaa can be any amino acid (More particularly, Xaa may be an amino acid selected from Leu, Thr, Met or Gln)
XCXXXNPSXC

SEQ ID NO: 57 - protein, 12 aa, Artificial Sequence
  Non-circular Rituximab mimotope
QDKLTQWPKWLE

SEQ ID NO: 58 - protein, 16 aa, Artificial Sequence
  QBend epitope sequence
ELPTQGTFSNVSTNVS

SEQ ID NO: 59 - protein, 42 aa, Artificial Sequence
  CD8 extracellular Stalk sequence
PAPRPPTPAPTIASQPLSLRPEACRPAAGGAVHTRGLDFACD

SEQ ID NO: 60 - protein, 82 aa, Artificial Sequence
  CD8 stalk, extracellular, TM and intracellular
PAPRPPTPAPTIASQPLSLRPEACRPAAGGAVHTRGLDFACDIYIWAPLAGTCGVLLLSLVITLYCNHRNRRRVCKCPRPVV

SEQ ID NO: 61 - protein, 6 aa, Artificial Sequence
  linker sequence
SGGGGS

SEQ ID NO: 62 - protein, 5 aa, Artificial Sequence
  linker sequence
SGGGS

SEQ ID NO: 63 - protein, 5 aa, Artificial Sequence
  linker domain
GGGGS

SEQ ID NO: 64 - protein, 30 aa, Artificial Sequence
  S1-Q-S2 linker sequence from RQR8
SGGGGSELPTQGTFSNVSTNVSPAKPTTTA

SEQ ID NO: 65 - protein, 20 aa, Artificial Sequence
  signal peptide (safety switch polypeptide)
MGTSLLCWMALCLLGADHAD

SEQ ID NO: 66 - protein, 21 aa, Artificial Sequence
  leader/signal sequence for CAR (modified CD8a leader)
MALPVTALLLPLALLLHAAAP

SEQ ID NO: 67 - protein, 21 aa, Artificial Sequence
  CD8a TM domain
IYIWAPLAGTCGVLLLSLVIT

SEQ ID NO: 68 - protein, 81 aa, Artificial Sequence
  modified CD8a hinge plus CD8a TM domain for CAR
FVPVFLPAKPTTTPAPRPPTPAPTIASQPLSLRPEASRPAAGGAVHTRGLDFADIYIWAPLAGTCGVLLLSLVITLYCNHR

SEQ ID NO: 69 - protein, 54 aa, Artificial Sequence
  modified CD8a hinge
FVPVFLPAKPTTTPAPRPPTPAPTIASQPLSLRPEASRPAAGGAVHTRGLDFAD

SEQ ID NO: 70 - protein, 82 aa, Artificial Sequence
  wild type CD8a hinge
FVPVFLPAKPTTTPAPRPPTPAPTIASQPLSLRPEACRPAAGGAVHTRGLDFACDIYIWAPLAGTCGVLLLSLVITLYCNHR

SEQ ID NO: 71 - protein, 43 aa, Artificial Sequence
  CD28 endodomain (2AA Transmembrane  - WV)
WVRSKRSRLLHSDYMNMTPRRPGPTRKHYQPYAPPRDFAAYRS

SEQ ID NO: 72 - protein, 115 aa, Artificial Sequence
  CD3? endodomain 1AA deletion compared to WT
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRSQL

SEQ ID NO: 73 - protein, 27 aa, Artificial Sequence
  CD28 Transmembrane domain
FWVLVVVGGVLACYSLLVTVAFIIFWV

SEQ ID NO: 74 - protein, 66 aa, Artificial Sequence
  CD28 hinge and transmembrane
IEVMYPPPYLDNEKSNGTIIHVKGKHLCPSPLFPGPSKPFWVLVVVGGVLACYSLLVTVAFIIFWV

SEQ ID NO: 75 - protein, 21 aa, Artificial Sequence
  wild type CD8a leader/signal sequence
MALPVTALLLPLALLLHAARP

SEQ ID NO: 76 - protein, 113 aa, Artificial Sequence
  WT hCD3? endodomain
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 77 - protein, 41 aa, Artificial Sequence
  WT CD28 endodomain
RSKRSRLLHSDYMNMTPRRPGPTRKHYQPYAPPRDFAAYRS

SEQ ID NO: 78 - protein, 42 aa, Artificial Sequence
  OX40 signalling domain
ALYLLRRDQRLPPDAHKPPGGGSFRTPIQEEQADAHSTLAKI

SEQ ID NO: 79 - protein, 42 aa, Artificial Sequence
  41BB signalling domain
KRGRKKLLYIFKQPFMRPVQTTQEEDGCSCRFPEEEEGGCEL

SEQ ID NO: 80 - protein, 38 aa, Artificial Sequence
  ICOS signalling domain
CWLTKKKYSSSVHDPNGEYMFMRAVNTAKKSRLTDVTL

SEQ ID NO: 81 - protein, 197 aa, Artificial Sequence
  TNFRSF25 signalling domain
TYTYRHCWPHKPLVTADEAGMEALTPPPATHLSPLDSAHTLLAPPDSSEKICTVQLVGNSWTPGYPETQEALCPQVTWSWDQLPSRALGPAAAPTLSPESPAGSPAMMLQPGPQLYDVMDAVPARRWKEFVRTLGLREAEIEAVEVEIGRFRDQQYEMLKRWRQQQPAGLGAVYAALERMGLDGCVEDLRSRLQRGP

SEQ ID NO: 82 - protein, 4 aa, Artificial Sequence
  Furin cleavage site
  misc_feature (2)..(3): Xaa can be any naturally occurring amino acid
RXXR

SEQ ID NO: 83 - protein, 4 aa, Artificial Sequence
  Furin cleavage site
RRKR

SEQ ID NO: 84 - protein, 286 aa, Artificial Sequence
  IL2RB
NCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 85 - protein, 94 aa, Artificial Sequence
  IL2RB truncated - Y510
NCRNTGPWLKKVLKCNTPDPSKFFSQLSSEHGGDVQKWLSSPFPSSSFSPGGLAPEISPLEVLERDKVTQLLPLNTDAYLSLQELQGQDPTHLV

SEQ ID NO: 86 - protein, 208 aa, Artificial Sequence
  IL2RB truncated - Y510 & Y392
NCRNTGPWLKKVLKCNTPDPSKFFSQLSSEHGGDVQKWLSSPFPSSSFSPGGLAPEISPLEVLERDKVTQLLDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 87 - protein, 407 aa, Artificial Sequence
  SFFV promoter
GTAACGCCATTTTGCAAGGCATGGAAAAATACCAAACCAAGAATAGAGAAGTTCAGATCAAGGGCGGGTACATGAAAATAGCTAACGTTGGGCCAAACAGGATATCTGCGGTGAGCAGTTTCGGCCCCGGCCCGGGGCCAAGAACAGATGGTCACCGCAGTTTCGGCCCCGGCCCGAGGCCAAGAACAGATGGTCCCCAGATATGGCCCAACCCTCAGCAGTTTCTTAAGACCCATCAGATGTTTCCAGGCTCCCCCAAGGACCTGAAATGACCCTGCGCCTTATTTGAATTAACCAATCAGCCTGCTTCTCGCTTCTGTTCGCGCGCTTCTGCTTCCCGAGCTCTATAAAAGAGCTCACAACCCCTCACTCGGCGCGCCAGTCCTCCGACAGACTGAGTCGGCCGG

SEQ ID NO: 88 - protein, 246 aa, Artificial Sequence
  3PB2 scFv
QVQLVQSGGGVVQPGGSLRVSCAASGVTLSDYGMHWVRQAPGKGLEWVAFIRNDGSDKYYADSVKGRFTISRDNSEKTVSLQMSSLRAEDTAVYYCAKNGESGPLDYWYLDLWGRGTLVTVSSGGGGSGGGGSGGGGSTDVVMTQSPSSLSASVGDRVTITCQSSLDISHYLNWYQQKPGKAPKLLIYDASNLETGVPSRFSGSGSGTHFTFTISSLQPEDFATYYCQQYDNLPLTFGGGTKLEIK

SEQ ID NO: 89 - protein, 22 aa, Artificial Sequence
  Linker sequence
LVTVSSGGGGSGGGGSGGGGST

SEQ ID NO: 90 - protein, 10 aa, Artificial Sequence
  Small SGGGGS linker
ETSGGGGSRL

SEQ ID NO: 91 - protein, 16 aa, Artificial Sequence
  Big SGGGGS linker
SGGGGSGGGGSGGGGS

SEQ ID NO: 92 - protein, 117 aa, Artificial Sequence
  Safety switch polypeptide
ACPYSNPSLCETSGGGGSRLCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 93 - protein, 123 aa, Artificial Sequence
  Safety switch polypeptide
ACPYSNPSLCSGGGGSGGGGSGGGGSCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 94 - protein, 137 aa, Artificial Sequence
  Polypeptide comprising a signal peptide and the amino acid sequence of SEQ ID NO. 92. (1xSGGGGS with leader)
MGTSLLCWMALCLLGADHADACPYSNPSLCETSGGGGSRLCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 95 - protein, 143 aa, Artificial Sequence
  Polypeptide comprising a signal peptide and the amino acid sequence of SEQ ID NO. 93. (3xSGGGGS with leader)
MGTSLLCWMALCLLGADHADACPYSNPSLCSGGGGSGGGGSGGGGSCPYSNPSLCSGGGGSPAPRPPTPAPTIASQPLSLRPEACRPAAGGAVHTRGLDFACDIYIWAPLAGTCGVLLLSLVITLYCNHRNRRRVCKCPRPVV

SEQ ID NO: 96 - protein, 10 aa, Artificial Sequence
  Spacer sequence
  MISC_FEATURE (4)..(9): Any one or all of amino acids 4-9 can either be present or absent
SGGGGGGGGS

SEQ ID NO: 97 - protein, 6 aa, Artificial Sequence
  Formula
  MISC_FEATURE (2)..(6): The amino acid residues 2-6 can be repeated one to eight times
SGGGGS

SEQ ID NO: 98 - protein, 30 aa, Artificial Sequence
  Linker
  MISC_FEATURE (2)..(30): Any one or all of amino acids 2-30 can either be present or absent
GGGGGGGGGGGGGGGGGGGGGGGGGGGGGG

SEQ ID NO: 99 - protein, 20 aa, Artificial Sequence
  Formula
  MISC_FEATURE (1)..(1): Ser may be absent
  MISC_FEATURE (2)..(17): The amino acid residues 2-17 can be repeated one to eight times
  MISC_FEATURE (3)..(9): Any one or all of amino acids 3-9 can either be present or absent
  MISC_FEATURE (11)..(17): Any one or all of amino acids 11-17 can either be present or absent
  MISC_FEATURE (18)..(20): Any one or all of amino acids 18-20 can either be present or absent
SGGGGGGGGSSSSSSSSGGG

SEQ ID NO: 100 - protein, 10 aa, Artificial Sequence
  Formula
  MISC_FEATURE (2)..(10): The amino acid residues 2-10 can be repeated one to eight times
  MISC_FEATURE (4)..(9): Any one or all of amino acids 4-9 can either be present or absent
SGGGGGGGGS

SEQ ID NO: 101 - protein, 9 aa, Artificial Sequence
  Formula
  MISC_FEATURE (1)..(9): The entire sequence of amino acids 1-9 can be repeated one to eight times
  MISC_FEATURE (3)..(8): Any one or all of amino acids 3-8 can either be present or absent
GGGGGGGGS

SEQ ID NO: 102 - protein, 12 aa, Artificial Sequence
  Formula
  MISC_FEATURE (1)..(9): The amino acid residues 1-9 can be repeated one to eight times
  MISC_FEATURE (3)..(8): Any one or all of amino acids 3-8 can either be present or absent
  MISC_FEATURE (10)..(12): Any one or all of amino acids 10-12 can either be present or absent
GGGGGGGGSGGG

(19) United States
(12) Patent Application Publication
(10) Pub. No.: US 2025/0144137 A1
(43) Pub. Date: May 8, 2025

(54) NUCLEIC ACID CONSTRUCTS FOR EXPRESSING POLYPEPTIDES IN CELLS

(71) Applicant: QUELL THERAPEUTICS LIMITED, London Greater London (GB)

(72) Inventor: Jenny MCGOVERN, London Greater London (GB)

(21) Appl. No.: 18/042,927

(22) PCT Filed: Aug. 27, 2021
(86) PCT No.: PCT/EP2021/073714

§ 371 Date: Feb. 24, 2023

(30) Foreign Application Priority Data
Aug. 27, 2020 (GB) 2013477.1

(51) Int. Cl. A61K 35/17; A61K 40/11; A61K 40/31; A61K 40/42; C07K 14/705; C07K 14/725; C07K 16/28; C12N 5/0783; C12N 15/86

(52) U.S. Cl. CPC A61K 35/17; A61K 40/11; A61K 40/31; A61K 40/421; C07K 14/7051; C07K 14/70517; C07K 14/70521; C07K 14/70596; C07K 16/2833; C12N 5/0637; C12N 15/86; A61K 2239/13; A61K 2239/17; A61K 2239/21; A61K 2239/25; C07K 2317/53; C07K 2317/565; C07K 2317/622; C07K 2319/02; C07K 2319/03; C12N 2510/00; C12N 2740/15043

(57) ABSTRACT

Provided herein is a nucleic acid molecule comprising 5′ to 3′ a first nucleotide sequence encoding a safety switch polypeptide comprising a suicide moiety; a second nucleotide sequence encoding FOXP3; and a third nucleotide sequence encoding a chimeric antigen receptor (CAR); particularly wherein said first, second, and third nucleotide sequences are separated by nucleotide sequences encoding self-cleavage sequences. Also provided are constructs, vectors and cells comprising the nucleic acid molecule, and methods and uses for expressing the encoded polypeptides in cells, particularly in immune cells useful in adoptive cell therapy (ACT).

### SEQUENCE LISTING

A Sequence Listing conforming to the rules of WIPO Standard ST.26 is hereby incorporated by reference. Said Sequence Listing has been filed as an electronic document via PatentCenter in ASCII TXT format. The electronic document, created on Sep. 12, 2023, is entitled “10414-101US1_ST25”, and is 81,378 bytes in size.

### FIELD

The present disclosure, and invention, relates to nucleic acids, including molecules and constructs, useful to express polypeptides in cells, particularly in immune cells useful in adoptive cell therapy (ACT). The nucleic acid encodes, in a single molecule, three components, namely a suicide moiety, the transcription factor FOXP3, and a chimeric antigen receptor (CAR), such that when the nucleic acid is expressed in a cell, the suicide moiety, the FOXP3 and the CAR are separately expressed as individual polypeptides in or on the cell.

### BACKGROUND

Adoptive cell therapy (ACT) has increasingly been used and tested in clinical application against a range of different conditions, including most notably malignant and infectious diseases where cytotoxic cells have been administered, and more recently regulatory T cell (Tregs) have been proposed for use to control unwanted immune responses based on their immunosuppressive effects.

T cells genetically engineered to recognise CD19 have been used to treat follicular lymphoma and ACT using autologous lymphocytes genetically-modified to express anti-tumour T cell receptors has been used to treat metastatic melanoma. The success of ACT in melanoma and EBV-associated malignancies spurred efforts to retarget effector T cells to treat other tumours, and T cells have been engineered to express T cell receptors (TCRs) or Chimeric antigen receptors (CARs) with new specificities.

CAR-modified T lymphocytes have been reported for immunotherapy of B-lineage malignancies, adult lymphoma, and disseminated melanoma.

Other types of immune cells are also being used or proposed for use in ACT, including for example, NK cells, including NK cells engineered to express CARs. More recently, regulatory T cells (Tregs) have been developed for ACT. Tregs have immunosuppressive function. They act to control cytopathic immune responses and are essential for the maintenance of immunological tolerance. The suppressive properties of Tregs can be exploited therapeutically, for example to improve and/or prevent immune-mediated organ damage in inflammatory disorders, autoimmune or allergic diseases or conditions, and in transplantation. As for effector immune cells, Tregs may analogously be genetically engineered to target a pre-determined molecule expressed on a cell, for example an antigen targeted via a CAR. Indeed, antigen specific Tregs offer the advantage of providing targeted immune suppression, and several groups have reported an increased suppressive ability as compared to polyclonal Tregs.

Thus, for ACT it is in many cases desirable to express a heterologous receptor, or targeting molecule, in a cell, particularly a chimeric receptor or CAR.

However, increasing efficacy of adoptive immunotherapy has been associated with reports of serious adverse events. Acute adverse events, such as cytokine storms, have been reported after infusion of engineered effector T-cells. In addition, chronic adverse events have occurred and others have been predicted by animal models. For example, effector T-cells re-directed to carbonic anhydrase IX (CAIX), an antigen expressed by renal carcinoma, produced hepatotoxicity in several patients due to unexpected CAIX expression on biliary epithelium. Native T-cell receptor transfer studies against melanoma have resulted in vitiligo and iritis in patients due to expression of target antigen on skin and iris. A graft-versus host disease (GvHD)-like syndrome due to TOR cross-pairing has been reported in mice after native TCR transfer. A lymphoproliferative disorder has been reported in an animal model after adoptive transfer with some CARs which incorporate co-stimulation. Finally, the risk of vector insertional mutagenesis is always present. While acute toxicities can be addressed by cautious dosing, chronic toxicities are likely to be cell dose independent.

Engineered and administered T cells can expand and persist for years after administration, and further Treg cells may in certain environments lose their suppressor phenotype and become T effector cells. In view of this, and the ever present risk of an adverse event after patient administration of any immunotherapy, it may in some cases, and for particular disease conditions, be desirable to include a safety mechanism to allow selective deletion of adoptively infused T-cells and other immune cells in the face of toxicity, or indeed after they have exerted their therapeutic effect.

Suicide genes enable selective deletion of transduced cells in vivo. Typically, a suicide gene encodes a suicide moiety which enables a cell to be eliminated, or targeted for elimination, as a safety measure, and thereby acts as a safety switch for the cell. Two suicide genes/moieties have been subjected to clinical testing: HSV-TK and iCasp9. Herpes Simplex Virus Thymidine kinase (HSV-TK) expression in T-cells confers susceptibility to ganciclovir. HSV-TK use is limited to clinical settings of profound immunosuppression such as haploidentical bone marrow transplantation as this viral protein is highly immunogenic. Further, it precludes the use of ganciclovir for cytomegalovirus treatment. Inducible Caspase 9 (iCasp9) can be activated by administration of a small molecule pharmaceutical (AP20187). Use of iCasp9 depends on availability of clinical grade AP20187. In addition, the use of an experimental small molecule in addition to a genetically engineered cell product may cause regulatory issues.

Other safety switches are being developed and WO2013/15339 has reported a polypeptide construct based on a minimal epitope from the antigen CD20, which is recognised by the lytic antibody Rituximab. Rituximab is an immunotherapeutic chimeric monoclonal antibody against the protein CD20, which is primarily found on the surface of B cells. When Ritiximab binds to CD20 it triggers cell death and thus it may be used to target and kill cells expressing CD20 on their cell surface. Peptides which mimic the epitope recognised by Rituximab (so-called mimotopes) have been developed, and these were used in WO2013/15339 as a suicide moiety in a combined suicide-marker polypeptide construct also comprising a CD34 minimal epitope as the marker moiety. Specifically, WO2013/15339 discloses a polypeptide termed RQR8, having the sequence set forth in SEQ ID NO. 1, which comprises two CD20 minimal epitopes, separated from one another by spacer sequences and an intervening CD34 marker sequence, and further linked to a stalk sequence which allows the polypeptide to project from the surface of a cell on which it is expressed.

It is thus further often desirable to include a safety switch in a cell which is engineered to express a CAR for ACT.

As noted above, Treg cells represent a category of cells useful for ACT in view of their suppressor function. Treg cells express FOXP3 and conventional T cells (Tcon cells) can be differentiated towards a regulatory phenotype ex vivo by expressing FOXP3 in those cells. Loss of FOXP3 expression is associated with a loss of suppressive function in regulatory T cells and a potential return to an effector phenotype. FOXP3 expression thus appears to be important for Treg function, and for maintenance of a Treg phenotype, and it is proposed herein further to express FOXP3 in immune cells, particularly Treg cells, intended for ACT.

### SUMMARY

The present inventors have developed nucleic acid molecules and constructs for co-expression of a CAR, a safety switch, and FOXP3 in cells, particularly in immune cells for ACT, more particularly Treg cells or precursors therefor. As noted above, it is proposed to express FOXP3 alongside the CAR and safety switch in order particularly to provide Treg cells with a stable Treg phenotype or to confer a Treg phenotype on the cell.

The nucleic acid molecules and constructs are designed to encode these three components within a single nucleic acid molecule, or construct, such that the encoded polypeptides may be produced in the cell as individual components i.e. as discrete entities. Thus, the three expressed components encoded by the nucleic acid molecule may be separately located, in or on the cell, as separate and distinct, or discrete, functional polypeptides. This may be achieved by encoding cleavage sequences in the nucleic acid molecule, in particular self-cleaving sequences, in between the nucleotide sequences encoding the respective components.

The inventors have surprisingly found that the order in which the three components are encoded in the nucleic acid molecule is important. In particular, it has been found that expression of the CAR, FOXP3, and safety switch as separate and individual polypeptides may be improved by encoding them in the following order 5′ to 3′ in the nucleic acid construct: safety switch polypeptide-FOXP3 polypeptide-CAR. When the components are encoded in a different order in comparative nucleic acid constructs, it has been found that the levels of the individual components are reduced as compared to the order of safety switch-FOXP3-CAR. Most notably, the level of FOXP3 production was unexpectedly and unpredictably very high with the construct safety switch-FOXP3-CAR. As shown in the Examples below, the construct herein enhanced the level of FOXP3 protein in transduced cells significantly more than did a comparable or control construct. The transduced FOXP3 conferred stability on Treg cells, the cells maintained their Treg phenotype, and further the elevated FOXP3 level did not negatively affect expansion or cell survival. Still further, the extremely high level of FOXP3 production observed was shown to be obtainable with different viral vectors.

Accordingly, in a first aspect, provided herein is a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR).

Particularly, the safety switch polypeptide, FOXP3 and the CAR may be expressed from the nucleic acid molecule as separate polypeptide entities.

In this respect, there is provided herein a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR);
   wherein said first, second, and third nucleotide sequences are separated by nucleotide sequences encoding self-cleavage sequences.

The self-cleavage sequences allow the expression, or production, of the safety switch polypeptide, the FOXP3 and the CAR as separate polypeptides.

In an embodiment the self-cleaving sequence, which may alternatively be referred to as a self-cleaving peptide, is a 2A or 2A-like cleavage sequence (or in other words, a 2A peptide, or 2A-like peptide).

In an embodiment the safety switch is the polypeptide RQR8, having an amino acid sequence as set forth in SEQ ID NO. 1, or a variant thereof, or similar polypeptide, as described further below.

The CAR may be directed to any desired target molecule, particularly to a target molecule expressed on a target cell. Suitable CARs are discussed below. In an embodiment the CAR is directed against an HLA antigen, for example HLA-A2. In a further embodiment, the CAR may not be directed against MHC II.

The nucleic acid molecule may be viewed as a nucleic acid construct which comprises the first, second and third coding nucleotide sequences. In a particular embodiment, the nucleic acid molecule may not encode any other polypeptide sequences (i.e. in an embodiment it does not comprise any coding nucleotide sequences other than the first, second and third coding nucleotide sequences of parts (i), (ii) and (iii) above. In other words, in an embodiment, the nucleotide sequences of (i), (ii) and (iiii) are the only, or the sole, coding sequences present in the nucleic acid molecule. Thus, alternatively viewed, the nucleic acid molecule may only encode a safety switch polypeptide, FOXP3 and a CAR. The nucleic acid molecule may be operably linked to a promoter to allow expression of the encoded polypeptides. Thus, the first, second and third nucleotide sequences may be operably linked to the same promoter. In this way, the three encoded polypeptide components may be expressed, as individual and separate components, from the same promoter. Particularly, the promoter may be a SFFV promoter.

Thus, in a further aspect, provided herein is an expression construct comprising the nucleic acid molecule as defined herein, wherein said first, second and third polynucleotide sequences are operably linked to the same promoter.

Alternatively viewed, the expression construct may be defined as comprising first, second and third nucleotide sequences as defined above, wherein said first, second, and third nucleotide sequences are separated by nucleotide sequences encoding cleavage sequences and wherein said first, second and third polynucleotide sequences are operably linked to the same promoter. The cleavage sequences allow expression of a safety switch polypeptide, FOXP3, and a CAR as separate polypeptides.

A third aspect provides a vector comprising the nucleic acid molecule or the expression construct as defined herein.

In an embodiment, the vector is a viral vector.

In a fourth aspect, provided herein is a cell comprising the nucleic acid molecule, the expression construct, or the vector as defined herein.

The cell is thus an engineered cell, or alternatively viewed, a cell which has been modified to introduce the nucleic acid molecule, construct or vector as defined herein. The cell recombinantly expresses the nucleic acid molecule. The cell may be a cell for production of the polypeptide, or for production of the nucleic acid molecule, construct or vector, for example for producing the vector. Accordingly, the cell may be a production host cell. Alternatively, the cell may be a cell intended for use in therapy, notably ACT. Thus, the cell may be an immune cell, particularly a T-cell and more particularly a Treg cell, e.g., a CD45RA+ Treg cell, or a progenitor or precursor thereof, including for example a stem cell, e.g. an induced pluripotent stem cell (iPSC). The CAR is expressed/localised on the surface of a cell intended for therapeutic use. The safety switch polypeptide may be expressed/localised in a cell, or on the surface of the cell, depending on the nature and type of the safety switch polypeptide. In an embodiment, the safety switch polypeptide is expressed/localised on the surface of the cell (separately to the CAR). FOXP3 is expressed/localised inside the cell. In particular, the FOXP3 is expressed in the nucleus and/or cytoplasm.

The invention further provides a cell population comprising a cell as defined herein.

In a fifth aspect provided herein is a pharmaceutical composition comprising a cell, cell population or a vector as defined herein. In an embodiment the cell expresses the CAR and the safety switch on its cell surface. The cell, cell population and the pharmaceutical composition as defined herein may be used in therapy, particularly ACT or gene therapy (where the composition comprises a vector).

Accordingly, a sixth aspect provided a cell, cell population or a pharmaceutical composition as defined herein for use in therapy.

A seventh aspect provides a cell, cell population or a pharmaceutical composition as defined herein for use in adoptive cell transfer therapy (ACT) or gene therapy.

An eighth aspect provides a cell, cell population or a pharmaceutical composition as defined herein for use in treating an infectious, neurodegenerative or inflammatory disease, or for inducing immunosuppression. The use, or immunosuppression, may be to suppress an unwanted or deleterious immune response. The use may be to treat inflammation, or an inflammatory condition, or condition involving or associated with inflammation.

A ninth aspect provides use of a cell or cell population as defined herein for the manufacture of a medicament for use in adoptive cell transfer therapy (ACT) or for use in treating an infectious, neurodegenerative or inflammatory disease, or for inducing immunosuppression.

A tenth aspect provides a method of therapy, particularly ACT, and more particularly for treating an infectious, neurodegenerative or inflammatory disease, or for inducing immunosuppression, wherein said method comprises administering to a subject, particularly a subject in need thereof, a cell or cell population as defined herein, particularly administering a therapeutically effective amount thereof.

In an embodiment, the cell, cell population or pharmaceutical composition, is provided herein for use in induction of tolerance to a transplant; treating and/or preventing graft-versus-host disease (GvHD), an autoimmune or allergic disease; to promote tissue repair and/or tissue regeneration; or to ameliorate inflammation. In particular, in such an embodiment, the cell may be a Treg cell.

Analogously, also provided is a method of inducing tolerance to a transplant; treating and/or preventing graft-versus-host disease (GvHD), an autoimmune or allergic disease; or to promote tissue repair and/or tissue regeneration; or to ameliorate inflammation, which method comprises the step of administering to said subject a cell, e.g. a Treg, a cell population, or a pharmaceutical composition as defined herein comprising a cell e.g. a Treg cell.

Such a method may comprise:
(i) isolation or provision of a Treg-enriched cell sample from a subject;
(ii) introduction into the Treg cells of a nucleic acid molecule, an expression construct or a vector as defined herein; and
(iii) administering the Treg cells from (ii) to the subject.

Further also provided herein is use of a cell, e.g. a Treg cell or cell population as defined herein in the manufacture of a medicament for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation.

For treatment of such conditions and disorders the CAR may be directed against an HLA antigen, e.g. HLA-A2.

An eleventh aspect provides a method of making a cell as defined herein which comprises the step of introducing into the cell (e.g. transducing or transfecting a cell with) a nucleic acid molecule, expression construct, or vector as defined herein, e.g. a Treg cell.

Such a method may further comprise a preceding step of isolating, enriching providing or generating a cell to be used in the method. Further, a cell may be isolated or enriched or generated after the step of introducing the nucleic acid molecule. For example, the nucleic acid molecule may be introduced into a precursor or progenitor cell, e.g. a stem cell, and the cell may then be induced or caused to differentiate, or change, into a desired cell type. For example, an iPSC cell may be differentiated into an immune effector cell (e.g. a Treg or other T cell) or a Tcon cell may be converted into a Treg cell, etc.

In a twelfth aspect, there is provided a method for deleting a cell according to the fourth aspect, which comprises the step of exposing the cells to an antibody having the binding specificity of Rituximab. In one aspect, the method may be an in vitro method.

Alternatively viewed, this aspect may comprise an antibody having the binding specificity of Rituximab for use in treating a subject to whom a cell of the fourth aspect as defined herein has been administered, to delete the cell.

Still further according to this aspect there is provided a kit, or combination product, comprising (a) a nucleic acid molecule, construct, vector or cell as defined herein and (b) an antibody having the binding specificity of Rituximab. The kit or product may be for use in ACT or gene therapy. In particular, the kit or product may be for use in treating a subject by ACT using the cell or manufacturing a cell of the invention for use, and thereafter deleting the cell from the subject. Alternatively, the kit may be for gene therapy using a vector to express the encoded polypeptides in vivo in cells of the subject. The antibody may be administered to the subject following administration of the cell or vector, for example after a period of time, or if the subject exhibits an unwanted or deleterious symptom or effect of the cell or gene therapy.

In an embodiment the antibody is Rituximab.

The invention may also provide a method for increasing the stability and/or suppressive function of a cell comprising the step of introducing a nucleic acid molecule, an expression construct or vector as provided herein into the cell.

In a further aspect, the invention provides a method of enhancing the expression of FOXP3 from a nucleic acid molecule encoding a chimeric antigen receptor (CAR), a safety switch and FOXP3 in a cell, comprising selecting a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a CAR,
and introducing said nucleic acid molecule into said cell.

As discussed above, expression of FOXP3 from a nucleic acid molecule encoding FOXP3, CAR and safety switch can be enhanced by positioning the encoding nucleotide sequences in the order of safety switch, FOXP3 and CAR within the nucleic acid molecule. Particularly, the expression of FOXP3 from the nucleic acid molecule may be enhanced as compared to expression from a nucleic acid molecule encoding a CAR, a safety switch and FOXP3 (preferably the same CAR, safety switch and FOXP3), wherein (i), (ii) and (iii) as defined above are positioned in a different order, for example as compared to a nucleic acid molecule comprising 5′ to 3′ (iii), (ii), (i), or comprising 5′ to 3′ (ii), (iii), (i).

In a further aspect, the present invention additionally provides a method of enhancing the expression of FOXP3 and of a chimeric antigen receptor from a nucleic acid molecule encoding a chimeric antigen receptor (CAR), a safety switch and FOXP3 in a cell, comprising selecting a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a CAR,
and introducing said expression construct into said cell.

In connection with this aspect, the expression of FOXP3 and CAR from the nucleic acid molecule is enhanced as compared to expression from a nucleic acid molecule encoding a CAR, a safety switch and FOXP3 (preferably, the same CAR, safety switch and FOXP3), wherein (i), (ii) and (iii) as defined above are positioned in a different order, for example as compared to a nucleic acid molecule comprising 5′ to 3′ (iii), (ii), (i), or comprising 5′ to 3′ (ii), (iii), (i).

In an additional aspect, the present invention provides the use of a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR) for expression of a suicide moiety, FOXP3 and a CAR within a cell, wherein expression of FOXP3 is enhanced.

In connection with this aspect, the present invention further provides the use of a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR) for expression of a suicide moiety, FOXP3 and a CAR within a cell, wherein expression of FOXP3 and the CAR is enhanced.

Alternatively viewed, the invention provides the use of a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR) for expression of a suicide moiety and a CAR and for enhanced expression of FOXP3 within a cell.

In a particular embodiment, the expression of the CAR is additionally enhanced.

### DESCRIPTION OF THE FIGURES

FIG. 1 shows the design of nucleic acid constructs made and tested, as described in the Examples below. Constructs I-VI are experimental constructs, wherein construct I is a construct according to the present invention and disclosure and constructs II-VI are comparative constructs, and constructs VII to X are control constructs comprising only 2 of the 3 components of the experimental (test) constructs. R=Safety Switch polypeptide; F=FOXP3; C=CAR.

FIG. 2 shows expression of experimental constructs and control constructs in T-effectors (Teff). Non-transduced cells (mock) and transduced cells were collected and stained for flow cytometry with fixable viability dye and antibodies against CD4, CD3, Dextramer, RQR8 and FOXP3. Top panel gated on Live/CD3+CD4+ cells. Panels show expression of Dextramer (x-axis). Bottom panels gated on Live/CD3+CD4+Dextramer+ cells and show expression of RQR8 (y-axis) and FOXP3 (x-axis). Lower panels represent expression in transduced cells only. Construct name references gene order, R=RQR8, F=FOXP3, C=CAR.

FIGS. 3A and B shows expression of experimental constructs in Tregs transduced with gamma retrovirus. After 15 days of expansion transduced cells were collected and stained for flow cytometry with fixable viability dye and antibodies against CD4, CD3, Dextramer, RQR8 and FOXP3. FACS plots in FIG. 3A show representative staining profile. Top panel shows Live/CD3+CD4+ cells. Panels show expression of Dextramer (x-axis). Bottom panels gated on Live/CD3+CD4+Dextramer+ cells and show expression of RQR8 (y-axis) and FOXP3 (x-axis). The graph in FIG. 3B is the result of Boolean analysis for each construct performed using Flowjo software depicting the proportion of cells expressing CAR, FOXP3 and RQR8, CAR and FOXP3 but not RQR8, CAR and RQR8 but not FOXP3 and CAR but not FOXP3 or RQR8. Representative of 3 independent donors.

FIG. 4 shows module expression in cells transduced with a lentiviral vector. Tregs were activated for 48 hours and transduced with unconcentrated lentiviral supernatant. 4 days later transduced cells and activated non-transduced cells (mock) were collected and stained for flow cytometry with fixable viability dye and antibodies against CD4, CD3, Dextramer, RQR8 and FOXP3. Upper panel is gated on Live/CD3+CD4+ cells and shows expression of Dextramer (x-axis). Lower panel is gated on Live/CD3+CD4+Dextramer+ cells and shows expression of RQR8 (y-axis) and FOXP3 (x-axis). Representative of n=3-4 donors. Lower panels represent expression in transduced cells only. Construct name references gene order, R=RQR8, F=FOXP3, C=CAR

FIG. 5 shows in A and B direct comparison of transduction with lentiviral or retroviral vectors for constructs I (FIG. 5A) and IV (FIG. 5B). Tregs from the same donor were activated for 48 hours and transduced with lentiviral or retroviral supernatant packaged with construct I. 4 days later cells were collected and stained for flow cytometry with fixable viability dye and antibodies against CD4, CD3, Dextramer, RQR8 and FOXP3. Table shows proportion of transgene expressing cells within the Live CD4+ population and mean fluorescence intensity (MFI). MFI of Dextramer and FOXP3 is taken from Dextramer+ cells. MFI of RQR8 is taken from RQR8+ cells.

FIG. 6 shows expression of FOXP3 in CAR negative cells (pale grey-non-transduced) and CAR+ cells (darker grey-transduced) for experimental constructs containing FOXP3 (construct I and VI) and control construct without FOXP3 (CVIII). FIG. 6A shows FACS plots and FIG. 6B presents histograms showing FOXP3 expression. For the construct I and VI histograms, the pale grey peaks can be visualized as the left hand peaks, and the darker grey peaks can be visualized as the right hand peaks. For construct VIII, the peaks are over laid.

FIG. 7 shows that exogenous FOXP3 expression prevents accumulation of FOXP3− CAR+ Tregs. Tregs were transduced with retroviral supernatant for indicated constructs. Tregs were collected and stained for flow cytometry with fixable viability dye and antibodies against CD4, CD3, Dextramer, RQR8 and FOXP3. Top panel shows FOXP3 (x-axis) and RQR8 (y-axis) expression on Dextramer+ cells 7 days after transduction. Lower panel shows FOXP3 (x-axis) and RQR8 (y-axis) expression on Dextramer+ cells 11 days after transduction following restimulation with anti-CD3/28 beads.

FIG. 8 shows Tregs activated for 48 hours and transduced with unconcentrated retroviral supernatant. 7 days later transduced cells and activated non-transduced cells (mock) were collected and stained for flow cytometry with fixable viability dye and antibodies against CD4, CD3, Dextramer, RQR8 and FOXP3. Upper panels gated on Live/CD3+CD4+ cells show expression of Dextramer (x-axis). Lower panels gated on Live/CD3+CD4+Dextramer+ cells. Plots show expression of RQR8 (y-axis) and FOXP3 (x-axis). Representative of n=7 donors. Construct name references gene order, R=RQR8, F=FOXP3, C=CAR

FIG. 9 shows Tregs activated for 48 hours and transduced with concentrated lentiviral supernatant. 7 days later transduced (Td) cells and activated non-transduced (ntd) cells (mock) were collected and stained for flow cytometry with fixable viability dye and antibodies against CD4, CD3, Dextramer, RQR8 and FOXP3. Gated on Live/CD3+CD4+ cells. Top panels show expression of Dextramer (x-axis), middle panels show expression of FOXP3 (x-axis) and RQR8 (x-axis) as indicated. Histograms show FOXP3 expression of non-transduced Tregs (pale grey) and Dextramer+ Tregs (darker grey) in the same sample. The pale grey peaks can be seen as the left hand peaks of each histogram and the darker grey peaks can be seen as the right hand peaks of each histogram, with the exception of the first histogram for mock cells which only has a pale grey peak.

FIGS. 10 A and B shows Treg cultures containing cells transduced with CAR+FOXP3 or CAR alone have similar overall expansion and survival. CD45RA+ Tregs were activated, transduced with retrovirus or lentivirus and grown following the protocol outlined in materials and methods. At days 5, 7, 9, 12 and 14 cells were counted using a multi-well disposable hemocytometer. The graph in FIG. 10A shows the fold expansion from day 0 of non-transduced Treg cell cultures (mock) and those transduced with constructs with (CI and CVI) and without (CVIII and CX) FOXP3. n=3-4. Error bars show standard deviation. On day 16 of culture RQR8+ cells were enriched using magnetic bead separation and cultured 1:1 with beads in the presence of decreasing concentrations of IL-2 for 7 days. Cells were stained with fixable viability dye and Dextramer. The graphs in FIG. 10B show Tregs transduced with (CI) and without (CVIII) FOXP3. The left y-axis indicates percentage of live cells (blue line with circular marked values) as determined by viability dye and the right Y axis indicates percentage of Dextramer+ cells (black line with square marked values). Representative of 3 experiments from Tregs transduced with retrovirus.

FIG. 11 shows transduced Tregs maintain a Treg phenotype. Exogenous transduced bulk and CD45RA+ Tregs were assayed by flow cytometry for indicated marker expression. In each sample transduced (TD) cells were identified as Dextramer+ and the remaining cells were considered non-transduced (NTD); mean fluorescence intensity (MFI) for each marker was determined in the TD and NTD cells. 2⁰ represents no change, 2¹ represents two fold increased expression Bars on graph represent the mean and error bars show standard deviation (n=3-4).

FIG. 12 shows transduction efficiency of Tregs when transduced with CI or with CVIII where either EFS or SFFV is used as the promoter. The cells were stained for Qbend (RQR8) and for Dextramer (CAR). Both EFS and SFFV resulted in similar transduction efficiency. Higher levels of transduction were seen in cells transduced with CVIII rather than CI, showing that transduction efficiency is more dependent on the construct size (2 genes rather than 3 genes).

FIG. 13 shows an activation assay for CAR Tregs, where activation of the cells using K562 cells expressing either HLA A1 or HLA A2, or using anti-CD3/CD28 beads was investigated. K562 cells expressing HLA A2 (i.e. expressing antigen recognized by the CAR used in this experiment) is capable of activating the CAR in cells transduced with both CI and CVIII (measured by CD69), when either the EFS or SFFV promoter is used. However, levels of activation are higher with the SFFV promoter. All cells can be activated using CD3/CD28 beads, which act as a positive control and activate cells using the endogenous TCR.

FIG. 14 shows CAR activation and proliferation of Tregs transduced with CI where either a EFS or SFFV promoter is used to control expression. Activation is investigated by incubation of cells with either K562 A1, K562 A2 or anti CD3/CD28 beads. The data shows that CAR-Tregs can proliferate after antigen specific stimulation and that SFFV enhances the proliferation rates after CAR activation.

FIG. 15 shows an in vivo assessment of Tregs transduced with CI and the ability of rituximab, when administered to the mouse model to deplete cells expressing an RQR8 safety switch in the liver. At day 15, transduced cells can be clearly seen in mice in the blood, spleen and liver in the absence of Rituximab treatment. However, in mice treated with Rituximab, cells are efficiently depleted.

FIG. 16 shows a Rituximab titration experiment where transduced Tregs were incubated with rabbit serum and increasing doses of Rituximab for 4 hours. The promoter used for expression of RQR8 was either EFS or SFFV. The data show that Rituximab can trigger cell death of CAR Tregs in a dose dependent manner and that SFFV provides increased sensitivity to Rituximab mediated depletion

### DETAILED DESCRIPTION

The present invention provides a nucleic acid molecule, or construct, which encodes polypeptides useful for expression in cells for ACT, specifically in the context of modifying a cell to express a CAR, and which allows the polypeptides to be encoded by a single nucleic acid molecule which may further encode self-cleavage sequences allowing the polypeptides to be expressed and/or produced as separate, or discrete components. By this it is meant that, although the polypeptides are encoded by a single nucleic acid molecule, through “cleavage” during or after translation at the encoded cleavage sites, they may be expressed or produced as separate polypeptides, and thus at the end of the protein production process in the cell, they may be present in the cell as separate entities, or separate polypeptide chains. As will be described further below, self-cleaving peptide sequences may be used, including particularly 2A and 2A-like peptides. Although described as “self-cleaving”, such peptides are believed to function by allowing ribosome skipping such that a peptide bond is not formed (skipped) at the C-terminus of the 2A sequence, leading to separation of the 2A sequence and the next polypeptide downstream of it. The term “cleavage” as used herein thus includes the skipping of peptide bond formation.

By “discrete” or “separate” polypeptides it is meant that the polypeptides are not linked to one another and are physically distinct. In other words they are expressed or produced in the cell as separate entities. Indeed, following expression, they are located in different, or separate cellular locations. The CAR, FOXP3 and safety switch polypeptide are thus ultimately expressed as single and separate components. The CAR is expressed as a cell surface molecule. The safety switch polypeptide may be expressed inside a cell, or on the cell surface. In a particular embodiment, the safety switch polypeptide and the CAR are expressed on the surface of a cell which is intended for ACT. The FOXP3 is expressed inside the cell, where it can exert its effect as a transcription factor to regulate cell development and/or activity, as described further below.

The safety switch polypeptide provides a cell in or on which it is expressed with a suicide moiety. This is useful as a safety mechanism which allows a cell which has been administered to a subject to be deleted should the need arise, or indeed more generally, according to desire or need, for example once a cell has performed or completed its therapeutic effect.

A suicide moiety possesses an inducible capacity to lead to cellular death, or more generally to elimination or deletion of a cell. An example of a suicide moiety is a suicide protein, encoded by a suicide gene, which may be expressed in or on a cell alongside a desired transgene, in this case the CAR, which when expressed allows the cell to be deleted to turn off expression of the transgene (CAR). A suicide moiety herein is a suicide polypeptide that is a polypeptide that under permissive conditions, namely conditions that are induced or turned on, is able to cause the cell to be deleted.

The suicide moiety may be a polypeptide, or amino acid sequence, which may be activated to perform a cell-deleting activity by an activating agent which is administered to the subject, or which is active to perform a cell-deleting activity in the presence of a substrate which may be administered to a subject. In a particular embodiment, the suicide moiety may represent a target for a separate cell-deleting agent which is administered to the subject. By binding to the suicide moiety, the cell-deleting agent may be targeted to the cell to be deleted. In particular, the suicide moiety may be recognised by an antibody, and binding of the antibody to the safety switch polypeptide, when expressed on the surface of a cell, causes the cell to be eliminated, or deleted.

The suicide moiety may be HSV-TK or iCasp9 as discussed above. However, it is preferred for the suicide moiety to be, or to comprise, an epitope which is recognised by a cell-deleting antibody or other binding molecule capable of eliciting deletion of the cell. In such an embodiment the safety switch polypeptide is expressed on the surface of a cell.

The term “delete” as used herein in the context of cell deletion is synonymous with “remove” or “ablate” or “eliminate” The term is used to encompass cell killing, or inhibition of cell proliferation, such that the number of cells in the subject may be reduced. 100% complete removal may be desirable but may not necessarily be achieved. Reducing the number of cells, or inhibiting their proliferation, in the subject may be sufficient to have a beneficial effect.

In particular, the suicide moiety may be a CD20 epitope which is recognised by the antibody Rituximab. Thus, in the safety switch polypeptide the suicide moiety may comprise a minimal epitope based on the epitope from CD20 that is recognised by the antibody Rituximab. More particularly, the polypeptide may comprise two CD20 epitopes R1 and R2 that are spaced apart by a linker L.

Accordingly, in an embodiment the safety switch polypeptide comprises a sequence having the formula;

R1-L-R2-St
wherein R¹ and R2 are Rituximab-binding epitopes;
St is a stalk sequence which, when the polypeptide is expressed at the surface of a cell, causes the R1 and R2 epitopes to be projected from the cell surface; and
L is a linker sequence.

Cells expressing a safety switch polypeptide comprising this sequence can be selectively killed using the antibody Rituximab, or an antibody having the binding specificity of Rituximab. The safety switch polypeptide is expressed on the cell surface and when the expressed polypeptide is exposed to or contacted with Rituximab, or an antibody with the same binding specificity, death of the cell ensues.

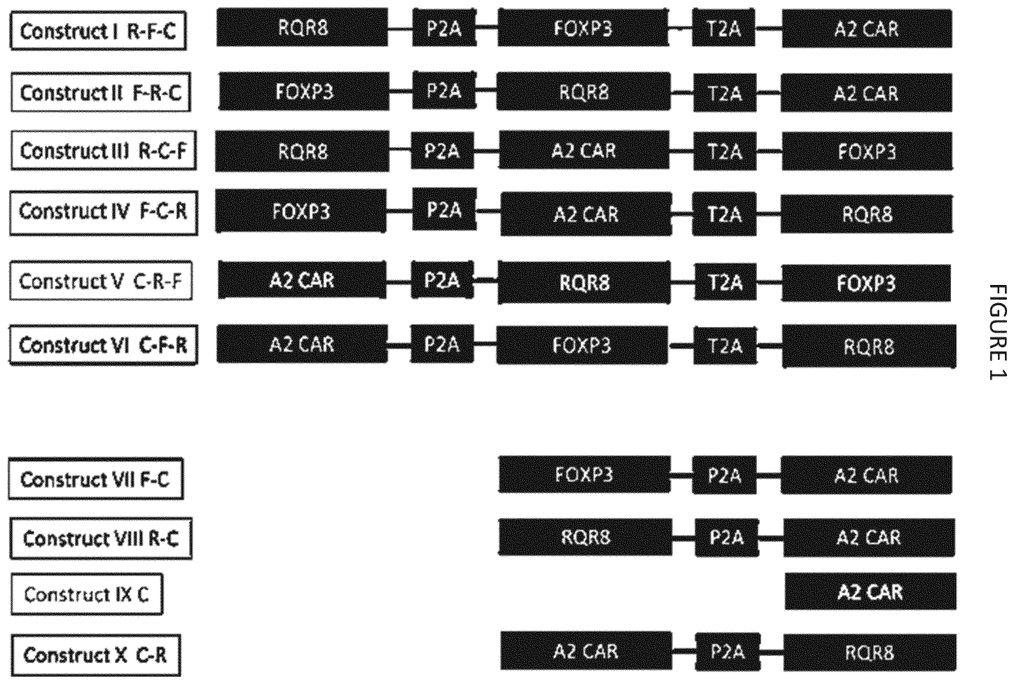

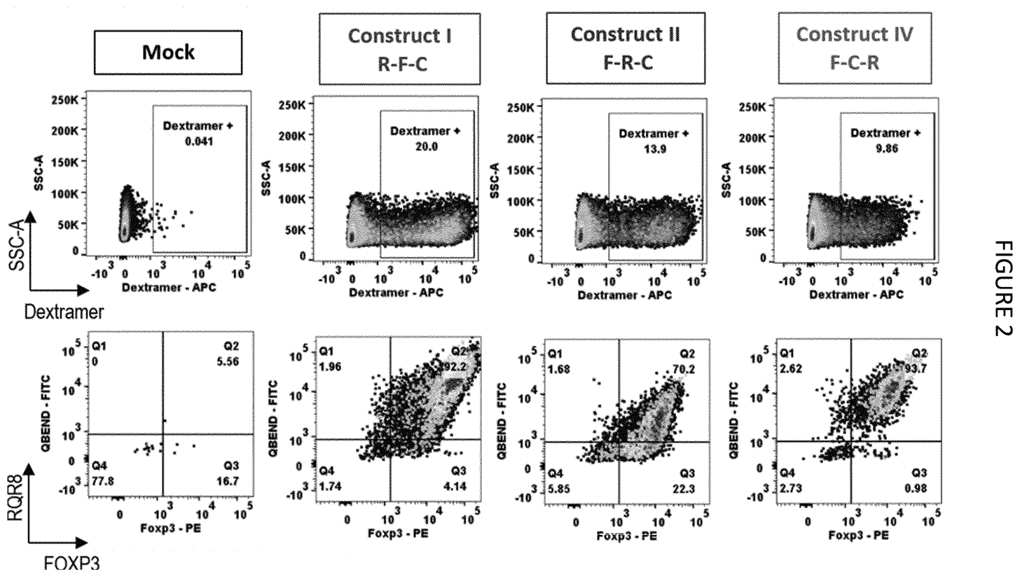

A Rituximab-binding epitope is an amino acid sequence which binds to the antibody Rituximab, or an antibody which has the binding specificity of Rituximab, in other words an antibody which binds to the same natural epitope as does Rituximab. Rituximab is a chimeric mouse/human monoclonal kappa IgG1 antibody which binds human CD20. The Rituximab-binding epitope sequence from CD20 is CEPANPSEKNSPSTQYC (SEQ ID NO. 33). Rituximab was first described in EP0669836 (hybridoma) and the heavy and light chain sequences are given in EP2000149 (see also Wang et al., Analyst, 2013, 138, 3058, which gives the heavy and light chain sequences in FIG. 1 thereof and Rituximab-CAS 17422-31-7, catolog number: B0084-061043, BOC Sciences). Reference may also be made to US 2009/0285795 A1, EP 1633398 A2, and WO 2005/000898. Rituximab and biosimilars thereof are widely available from various commercial sources around the world.

R1 and R2 may thus be any peptide which binds to, or in other words which is capable of binding to, Rituximab. As well as the natural epitope in the context of CD20, various peptides are known, and have been reported, which bind to Rituximab, or more particularly which mimic the natural epitope. R1 and R2 may accordingly be a mimotope of the Rituximab epitope.

Such mimotopes are described for example in Perosa et al (2007, J. Immunol 179:7967-7974) which discloses a series of cysteine-constrained 7-mer cyclic peptides, which bear the antigenic motif recognised by Rituximab but have different motif-surrounding amino acids. Perosa describe eleven peptides with SEQ ID NOs. 34 to 44 as shown in Table 1 below. In the Table the amino acids flanking the motif are shown in lower case, and the motif is shown in upper case. It has been determined that the initial amino acid “a” may be removed from the peptide and a functional epitope (or mimotope) may be retained. Peptides of SEQ ID NOs. 45 to 55 lacking the initial “a” are also shown in Table 1.

TABLE 1

| Perosa peptide | | |
|---|---|---|
| designation | Sequence | Modified sequence |
| R15-C | acPYANPSLc (SEQ ID No. 34) | cPYANPSLc (SEQ ID No. 45) |
| R3-C | acPYSNPSLc (SEQ ID No. 35 | cPYSNPSLc (SEQ ID No. 46) |
| R7-C | acPFANPSTc (SEQ ID No. 36) | CPFANPSTc (SEQ ID No. 47 |
| R8-, R12-, | acNFSNPSLc (SEQ ID No. 37 | cNFSNPSLc (SEQ ID No. 48) |
| R18-C | | |
| R14-C | acPFSNPSMc (SEQ ID No. 38) | CPFSNPSMc (SEQ ID No. 49) |
| R16-C | acSWANPSQc (SEQ ID No. 39) | cSWANPSQc (SEQ ID No. 50) |
| R17-C | acMFSNPSLc (SEQ ID No. 40) | CMFSNPSLc (SEQ ID No. 51) |
| R19-C | acPFANPSMc (SEQ ID No. 41) | CPFANPSMc (SEQ ID No. 52) |
| R2-C | acWASNPSLc (SEQ ID NO. 42) | cWASNPSLc (SEQ ID NO. 53) |
| R10-C | acEHSNPSLc (SEQ ID No. 43) | CEHSNPSLc (SEQ ID No. 54) |
| R13-C | acWAANPSMc (SEQ ID No. 44) | cWAANPSMc (SEQ ID No. 55) |

A circular (or cyclic) mimotope of the Rituximab epitope which may be used as R1 and/or R2 according to the invention may be represented by the consensus amino acid sequence of SEQ ID NO. 56:

X1-C-X2-X3-(A/S)-N-P-S-X4-C

wherein X1 is A or absent, and X2, X3 and X4 are any amino acid.
More particularly, X2 may be an amino acid selected from P, N, S, M, W or E; X3 may be an amino acid selected from Y, F, W, A, or H; and X4 may be an amino acid selected from L, T, M or Q.

Non-circular (or non-cyclic) peptide mimotopes of the Rituximab epitope have also been developed. Li et al (2006 Cell Immunol 239:136-43) also describe mimotopes of Rituximab, including a peptide with the sequence QDKLTQWPKWLE (SEQ ID NO. 57).

The polypeptide may comprise Rituximab-binding epitopes R1 and R2 which each independently comprise an amino acid sequence selected from the group consisting of SEQ ID NO. 34 to 57, or a variant thereof which retains Rituximab-binding activity.

The two epitopes R1 and R2 may be the same or different, but in one preferred embodiment they are the same.

In an embodiment, R1 and R2 each consist essentially of, or alternatively each consist, of an amino acid sequence selected from the group consisting of SEQ ID NO. 33 to 57, or a variant thereof which retains Rituximab-binding activity.

In a representative embodiment, the polypeptide may comprise Rituximab-binding epitopes R1 and R2 comprising, consisting essentially of, or consisting of, the amino acid sequence shown as SEQ ID NO. 46 or a variant thereof which retains Rituximab-binding activity.

A variant Rituximab-binding epitope may be based on the sequence selected from the group consisting of SEQ ID NOs. 33-55 or 57 but comprises one or more amino acid mutations, such as amino acid insertions, substitutions or deletions, relative to the sequence, provided that the epitope retains Rituximab-binding activity. In particular, the sequence may be truncated at one or both terminal ends by, for example, one or two amino acids.

Deliberate amino acid substitutions may be made on the basis of similarity in polarity, charge, solubility, hydrophobicity, hydrophilicity, and/or the amphipathic nature of the residues as long as Rituximab-binding activity of the epitope is retained. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to Table 2 below:

TABLE 2

| | | |
|---|---|---|
| ALIPHATIC | Non-polar | G A P |
| | | I L V |
| | Polar-uncharged | C S T M |
| | | N Q |
| | Polar-charged | D E |
| | | K R |
| AROMATIC | | H F W Y |

Amino acids in the same block in the second column and in the same line in the third column may be substituted for each other:

The Rituximab-binding epitope may, for example, contain 3 or fewer, 2 or fewer or 1 amino acid mutation(s) compared to the sequence selected from the group consisting of SEQ ID NOs. 33-57.

A variant of a Rituximab-binding epitope may comprise or consist of an amino acid sequence having at least 75% sequence identity to any one of SEQ ID NOs. 33 to 55 or 57, more particularly at least 80, 85, 90, 95, 96, 97, 98 or 99% sequence identity thereto.

Where two identical (or similar) Rituximab-binding amino acid sequences are used, it may be advantageous to use different nucleotide sequences to encode the two R epitopes. In many expression systems, homologous sequences can result in undesired recombination events. Using the degeneracy of the genetic code, alternative codons may be used to achieve nucleotide sequence variation without altering the protein sequence thereby preventing homologous recombination events.

The linker sequence L may be any amino acid sequence which functions to link, or connect, the two “R” epitopes together, such that they may be recognised and bound by the antibody. In particular, the R epitopes are spaced apart so that they are each bound by a separate antibody molecule. Thus, the linker sequence L is of a length which does not permit the two R epitopes to bind to the same antibody molecule at the same time, or put another way, the linker L is too long for the safety switch polypeptide to bind to both antigen-binding sites of a Rituximab molecule simultaneously. The linker L is of a length such that the two R epitopes can each bind to a separate Rituximab molecule.

The linker sequence may perform simply the function of connecting and spacing apart the R epitopes. Alternatively, or additionally, the linker sequence may comprise further functional domains or sequences. For example, it may contain a marker sequence. The marker sequence may be an epitope recognised by an antibody (in this context it will be understood that this is a different epitope to that recognised by Rituximab). The marker sequence may in itself function as a spacer sequence, and so separate spacer sequences in addition to the marker sequence may not be required.

As noted above, the suicide constructs of WO2013/15339 comprise minimal CD20 epitopes (specifically based on SEQ ID NOs. 34-44 and 57 as set out above) spaced apart by a linker which comprises a CD34 marker sequence, further linked to a stalk sequence. Particularly, the suicide polypeptides of WO2013/15339 are defined therein as having the general formula St-R1-S1-Q-S2-R2 wherein R¹ and R2 are Rituximab-binding epitopes, St, is a stalk sequence, S1 and S2 are spacer sequences; and Q is a CD34 epitope having the sequence of SEQ ID NO. 58 (ELPTQGTFSNVSTNVS) or a variant thereof.

The epitope of SEQ ID NO. 58 is recognised by the monoclonal antibody QBEnd10. This is important as the QBEnd10 antibody is used in the Miltenyi CliniMACS magnetic cell selection system, which is widely used for isolation of cells in clinical settings. Accordingly, the inclusion of the Q epitope as a marker allows cells which have been modified to express this polypeptide readily to be selected using a commonly available selection system.

In an embodiment the safety switch polypeptide may consist of or comprise a polypeptide as disclosed in WO2013/15339. In such an embodiment in the formula R1-L-R2-St above L may be defined as:

S1-Q-S2,
wherein Q comprises (e.g. is) a CD34 epitope having the sequence of SEQ ID NO. 58, or a variant thereof which has QBEnd10 binding activity; and
S1 and S2 are optional spacer sequences which may be the same or different.

As noted above, the linker L functions to space apart the two R epitopes so that they can bind Rituximab in a manner which enables it to elicit its effect as a cell-deleting agent, i.e. they can each bind to a separate Rituximab molecule. Thus, the length of S1-Q-S2, or the distance between R1 and R2, is such the safety switch polypeptide cannot bind to both antigen-binding sites of a Rituximab molecule simultaneously.

The spacer sequences S1 and S2 may have a combined length of at least about 10 amino acids.

The distance between R1 and R2 may be more than 76.57A. For example, the length and configuration of the spacer sequences may be such that the distance between R1 and R2 is at least 78, 80 or 85 Å. For the purposes of this calculation, the molecular distance between separate amino acids in a linear back bone can be assumed to be approximately 3A per amino acid. The spacer sequence(s) may be substantially linear. A spacer sequence may have the sequence of a flexible linker sequence as defined below, particularly a sequence which comprises or consists of serine and glycine residues, as described further below. The spacer sequence(s) may have the general formula S-(G)n-S (SEQ ID NO: 96) where S is serine, G is Glycine and n is a number between 2 and 8. The, or each, linker may comprise or consist of the sequence SGGGS (SEQ ID NO. 62). However, other spacer sequences may also be used. The amino acid sequence of the spacer is not critical. The combined length of the Q epitope and spacer(s) (i.e. the length of the S1-Q-S2 linker sequence) may be at least 28 amino acids.

The variant of SEQ ID NO. 58 which retains QBEnd10 binding activity may be an amino acid sequence having at least 80% sequence identity to SEQ ID NO. 58, for example at least 85, 90, or 95, 96, 97, 98 or 99% sequence identity to SEQ ID NO. 58.

The QBEnd10-binding variant of SEQ ID NO. 58 may contain one or more amino acid sequence modifications, e.g. amino acid substitutions, additions, deletions or insertions, including conservative substitutions as discussed above.

Antibody QBEnd10 is available from various sources including Abcam, ThermoFisher, Santa Cruz Biotechnology and Bio-Rad. Details of the antibody are available in EP3243838A1 and Chia-Yu Fan et al. Biochem Biophys Rep. 2017 March; 9:51-60. Methods for determining binding activity to QBEnd10 may readily be performed according to techniques well known in the art.

In other embodiments the linker sequence L does not comprise a marker sequence. In an embodiment it does not comprise a QBEnd10-binding epitope as defined and described above.

In such embodiments, the linker sequence may, as noted above, be any amino acid sequence which serves the function of spacing apart the R epitopes, allowing them each to bind to a separate Rituximab antibody molecule. The nature of the sequence, in terms of its amino acid composition and/or sequence of amino acids may be varied and is not limited. However, in an embodiment the linker may be a flexible linker. It may thus comprise or consist of amino acids known to confer a flexible character to the linker (as opposed to a rigid linker).

Flexible linkers are a category of linker sequences well known and described in the art. Linker sequences are generally known as sequences which may be used to link, or join together, proteins or protein domains, to create for example fusion proteins or chimeric proteins, or multifunctional proteins or polypeptides. They can have different characteristics, and for example may be flexible, rigid or cleavable. Protein linkers are reviewed for example in Chen et al., 2013, Advanced Drug Delivery Reviews 65, 1357-1369, which compares the category of flexible linkers with those of rigid and cleavable linkers. Flexible linkers are also described in Klein et al., 2014, Protein Engineering Design and Selection, 27 (10), 325-330; van Rosmalen et al., 2017, Biochemistry, 56, 6565-6574; and Chichili et al., 2013, Protein Science, 22, 153-167.

A flexible linker is a linker which allows a degree of movement between the domains, or components, which are linked. They are generally composed of small non-polar (e.g. Gly) or polar (e.g. Ser or Thr) amino acid residues. The small size of the amino acids provides flexibility and allows for mobility of the connected parts (domains or components). The incorporation of polar amino acids can maintain the stability of the linker in aqueous environments by forming hydrogen bonds with water molecules.

The most commonly used flexible linkers have sequences primarily composed of Ser and Gly residues (so-called “GS linkers”). However, many other flexible linkers have also been described (see Chen et al, 2013, supra, for example), which may contain additional amino acids such as Thr and/or Ala, and/or Lys and/or Glu which may improve solubility. Any flexible linker known and reported in the art may be used.

The use of GS linkers, or more particularly GS (“Gly-Ser”) domains in linkers, may allow the length of the linker readily to be varied by varying the number of GS domain repeats, and so such linkers represent one suitable class of linkers. However, flexible linkers are not limited to those based on “GS” repeats, and other linkers comprising Ser and Gly residues dispersed throughout the linker sequence have been reported, including in Chen et al., supra.

In one embodiment, the linker sequence comprises at least one Gly-Ser domain composed solely of Ser and Gly residues. In such an embodiment, the linker may contain no more than 15 other amino acid residues, preferably no more than 14, 13, 12, 11, 10, 9, 8, 6, 7, 5, or 4 other amino acid residues.

The Gly-Ser domain may have the formula:

(S)q-[(G)m-(S)m]n-(G)p (SEQ ID NO: 99)
wherein q is 0 or 1; m is an integer from 1-8; n is an integer of at least 1 (e.g. from 1 to 8, or more particularly 1 to 6); and p is 0 or an integer from 1 to 3.

More particularly, the Gly-Ser domain may have the formula;

(i) S-[(G)m-S]n (SEQ ID NO: 100);

(ii) [(G)m-S]n (SEQ ID NO: 101); or

(iii) [(G)m-S]n-(G)p (SEQ ID NO: 102)
wherein m is an integer from 2-8 (for example 3-4); n is an integer of at least 1 (for example from 1 to 8, or more particularly 1 to 6); and p is 0 or an integer from 1 to 3.

In a representative example, the Gly-Ser domain may have the formula:

(SEQ ID NO: 97)
S-[G-G-G-G-S]n

wherein n is an integer of at least one (preferably 1 to 8, or 1-6, 1-5, 1-4, or 1-3). In the formula above, the sequence GGGGS is SEQ ID NO. 63.

Representative exemplary linker sequences are listed below:

(SEQ ID NO. 90)
ETSGGGGSRL
((SEQ ID NO. 91)
SGGGGSGGGGSGGGGS
S(GGGGS)₁₋₅
(where GGGGS is SEQ ID NO. 63)

In other embodiments the linker sequence is not a flexible linker sequence.

In the safety switch polypeptide, the function of the linker L is to connect R1 to R2. The linker may connect R1 and R2 directly, that is the C-terminus of R1 to the N-terminus of R2. Thus, the polypeptide may not contain any other component or sequence between R1 and R2 other than the linker sequence L. It will be understood that since the polypeptide is to be expressed on the surface of the cell and since R1 is connected to R2 such that both R1 and R2 are to be expressed on the surface of the cell, the linker L is not a cleavable linker.

Although the length of the linker is not critical, it may in some embodiments be desirable to have a shorter linker sequence. For example, the linker sequence may have a length of no more than 25, preferably no more than 24, 23, 22 or 21 amino acids.

In other embodiments, a longer linker sequence may be desired, for example composed of, or comprising, multiple repeats of a GS domain, and/or comprising a marker sequence, such as an epitope as discussed above.

In some embodiments the linker may be from any one of 2, 3, 4, 5 or 6 to any one of 24, 23, 22 or 21 amino acids in length. In other embodiments it may be from any one of 2, 3, 4, 5 or 6 to any one of 21, 20, 19, 18, 17, 16, or 15 amino acids in length. In other embodiments it may be intermediate between these ranges, from example from 6 to 21, 6 to 20, 7 to 20, 8-20, 9-20, 10-20, 8-18, 9-18, 10-18, 9-17, 10-17, 9-16, 10-16 etc. It may accordingly be within a range made up from any of the integers listed above.

The safety switch polypeptide may comprise a stalk sequence (St) which, when the polypeptide is expressed at the surface of a target cell, causes the R epitopes to be projected away from the surface of the target cell.

The stalk sequence causes the R epitopes to be sufficiently distanced from the cell surface to facilitate binding of, for example, Rituximab or an equivalent antibody. The stalk sequence elevates the epitopes from the cell surface.

The stalk sequence may be a substantially linear amino acid sequence. The stalk sequence may be sufficiently long to distance the R epitopes from the surface of the target cell but not so long that its encoding sequence compromises vector packaging and transduction efficiency. The stalk sequence may, for example be between 30 and 100 amino acids in length.

The stalk sequence may be approximately 40-50 amino acids in length. The stalk sequence may be highly glycosylated.

The stalk sequence may comprise a linker sequence which links or connects it to the epitope R2 in the formula above.

A wide range of proteins are known which are expressed on the surface of mammalian cells and which can be used to provide, or as the basis for, a stalk sequence herein. Such surface-expressed proteins comprise natural sequences which can be used as, or to derive, a stalk sequence. For example, the extracellular domain (ECD) of such a protein may be used as a stalk sequence, or the extracellular and transmembrane (TM) domains, or the extracellular and transmembrane domains (ECD and TMD) with an intracellular domain (ICD) which may serve as an intracellular anchor to hold the stalk in the membrane and allow it to project from the cell surface.

Such proteins include CD27, CD28, CD3 epsilon, CD3z, CD45, CD4, CD5, CD8, CD9, CD16, CD18, CD22, CD33, CD37, CD64, CD80, CD86, CD134, CD137, CD152, CD154, CD278, CD279, IgG1, or IgG2.

Thus, the stalk sequence St may comprise an optional linker sequence which connects it to R2, an extracellular domain, an optional transmembrane domain, and an optional intracellular domain.

In an embodiment the stalk sequence may comprise a linker sequence which connects it to R2, an extracellular domain, a transmembrane domain, and an intracellular domain.

The stalk sequence, or the extracellular domain thereof, may comprise or be approximately equivalent in length to the sequence:

(SEQ ID NO. 59)
PAPRPPTPAPTIASQPLSLRPEACRPAAGGAVHTRGLDFACD,

which is the extracellular sequence from CD8.

As noted above, the stalk sequence may additionally comprise a transmembrane domain, optionally together with an intracellular domain, which may serve as an intracellular anchor sequence. The transmembrane domain and intracellular domain may be derived from the same protein as the extracellular domain or it/they may be derived from a different protein. The transmembrane domain and intracellular domain may be derivable from CD8.

The stalk sequence St may comprise an extracellular stalk sequence, a transmembrane domain, and an intracellular domain derived from CD8.

A CD8 stalk sequence which comprises a transmembrane domain and an intracellular anchor may have the following sequence:

(SEQ ID NO. 60)
PAPRPPTPAPTIASQPLSLRPEACRPAAGGAVHTRGLDFACDIYIWAPL
AGTCGVLLL SLVITLYCNHRNRRRVCKCPRPVV,

or a sequence which hast at least 75%, particularly at least 80, 85, 90, 95, 96, 97, 98 or 99%, sequence identity thereto.

Within this sequence, the underlined portion corresponds to the extracellular CD8a stalk; the central portion corresponds to the transmembrane domain; and the portion in bold corresponds to the intracellular domain.

The linker sequence in a stalk sequence may be a linker as described above. In particular, it may be a linker sequence which comprises or consists of Ser(S) and/or Gly (G) residues. The linker sequence(s) may be substantially linear. In the context of the stalk, the linker sequence may be a shorter sequence. For example, the linker sequence(s) may have the general formula:

S-(G)n-S (SEQ ID NO: 96)

where n is a number between 2 and 8.

The linker may comprise or consist of the sequence SGGGGS (SEQ ID NO. 61).

Representative exemplary embodiments of the safety switch polypeptide include polypeptides comprising Rituximab binding epitopes of SEQ ID NO. 46 and/or SEQ ID NO. 35 or a sequence having at least 80% sequence identity therewith, linked via a linker to a stalk sequence comprising extracellular, transmembrane and intracellular sequences derived from CD8. In particular, the stalk sequence may have the sequence of SEQ ID NO. 60, or sequence with at least 80% sequence identity therewith. The linker L between R1 and R2 may be any of the linkers discussed above. For example, it may be flexible linker as described above, or it may be a linker comprising a QBEnd epitope as discussed above. In an embodiment the linker may have the sequence:

(SEQ ID NO. 63)
SGGGGSELPTQGTFSNVSTNVSPAKPTTTA,

or a sequence with at least 80% sequence identity therewith. The epitope Q is indicated in bold in the above sequence, and the flanking sequences represent the spacers S1 and S2, respectively. The stalk sequence may comprise a linker sequence which connects to R2. The linker sequence in the stalk may be SGGGS (SEQ ID NO. 62).

Accordingly, the safety switch polypeptide may comprise or consist of the amino acid sequence shown as SEQ ID NO. 1, or a sequence having at least 75%, particularly at least 80, 85, 90, 95, 96, 97, 98 or 99% sequence identity thereto.

In other embodiments the safety switch polypeptide may comprise or consist of the amino acid sequence set out in SEQ ID NO. 92 or SEQ ID NO. 93, or a sequence having at least 75%, particularly at least 80, 85, 90, 95, 96, 97, 98 or 99% sequence identity thereto.

(SEQ ID NO. 92)
ACPYSNPSLCETSGGGGSRLCPYSNPSLCSGGGGSPAPRPPTPAPTIASQ
PLSLRPEACRPAAGGAVHTRGLDFACDIYIWAPLAGTCGVLLLSLVITLY
CNHRNRRRVCKCPRPVV
(SEQ ID NO. 93)
ACPYSNPSLCSGGGGSGGGGSGGGGSCPYSNPSLCSGGGGSPAPRPPTPA
PTIASQPLSLRPEACRPAAGGAVHTRGLDFACDIYIWAPLAGTCGVLLLS
LVITLYCNHRNRRRVCKCPRPVV

SEQ ID NOs. 92 and 93 each respectively comprise a Rituximab-binding epitope of SEQ ID NO. 35 linked via a linker of SEQ ID NO. 90 or 91 respectively to a Rituximab binding epitope of SEQ ID NO. 46.

The polypeptide may also comprise, or be expressed with, a signal peptide at the amino terminus (otherwise known as a leader sequence). A number of different signal sequences are known and reported in the art and it would be a matter of routine to select a signal peptide. The signal peptide may, for example, comprise or consist of the sequence shown as SEQ ID NO. 65 (MGTSLLCWMALCLLGADHAD).

A polypeptide comprising such a signal peptide and the amino acid sequence of SEQ ID NO. 1 is represented by SEQ ID NO. 10. A polypeptide comprising such a signal peptide and the amino acid sequence of SEQ ID NO. 92 is represented by SEQ ID NO. 94. A polypeptide comprising such a signal peptide and the amino acid sequence of SEQ ID NO. 93 is represented by SEQ ID NO. 95.

Once the polypeptide is expressed by the target cell (that is the cell into which a nucleic acid molecule comprising a nucleotide sequence encoding the polypeptide is introduced), it is processed in the cell for translocation to the cell surface, and the signal peptide is cleaved, resulting in the mature safety switch polypeptide product.

The safety switch polypeptide may comprise or consist of a variant of the sequence shown as SEQ ID NO. 1, 92 or 93 which has at least 75% (e.g. at least 80% or 90%) identity with the sequence shown as SEQ ID NO. 1, 92 or 93 as long as it retains the functional activity of the SEQ ID NO. 1, 92 or 93 polypeptide. For example, the variant sequence should (i) bind Rituximab and (ii) when expressed on the surface of a cell, induce killing of the cell in the presence of Rituximab. Further, in the case of a variant of SEQ ID NO. 1, the variant sequence should bind to the antibody QBEnd10.

Sequence identity comparisons may be conducted by eye or with the aid of readily available sequence comparison programs, such as the GCG Wisconsin Bestfit package.

In an embodiment the safety switch polypeptide consists only of the elements R1, L, R2 and St as set out and described above. However, in other embodiments the polypeptide may additionally comprise a further sequence, or domain. For example, if the linker L does not contain a marker sequence or if more than one marker is desirable, then a separate maker sequence may be included elsewhere, for example a marker sequence may be included the stalk sequence, or may be introduced between the stalk and R2.

Safety switch polypeptides are described in PCT/EP2021/064053, the contents of which are incorporated herein by reference. Any of the safety switch polypeptides described in that document may be used herein.

The nucleic acid molecule is designed to increase FOXP3 expression in cells (e.g. Tregs) by introducing into the cells a nucleotide sequence encoding FOXP3 (the second nucleotide sequence), which term is synonymous with the term “a FOXP3 polypeptide”. The nucleic acid molecule, and constructs and vectors containing it, thus provide a means for increasing FOXP3 in a cell, e.g. in a Treg or a CD4+ cell. Further, as described previously, the order of the nucleotide sequences encoding the safety switch, FOXP3 and the CAR within the nucleic acid molecule of the invention additionally results in an enhanced expression of FOXP3 within a cell from the nucleic acid molecule. The nucleic acid molecules of the invention therefore provide for expression of FOXP3 (and of the safety switch and CAR) and thus result in an increased expression level of FOXP3 within the cell, but the order of the nucleotide sequences further results in an enhancement of FOXP3 expression over other nucleic acid molecules encoding the same three polypeptide products but from nucleotide sequences presented in a different order within the nucleic acid molecule. Thus, the order of the nucleotides sequences encoding the safety switch, FOXP3 and the CAR within the nucleic acid molecule of the invention provides an optimally increased expression level of FOXP3 within a cell into which the nucleic acid molecule is introduced.

“FOXP3” is the abbreviated name of the forkhead box P3 protein. FOXP3 is a member of the FOX protein family of transcription factors and functions as a master regulator of the regulatory pathway in the development and function of regulatory T cells. “FOXP3” as used herein encompasses variants, isoforms, and functional fragments of FOXP3.

“Increasing FOXP3 expression” means to increase the levels of FOXP3 mRNA and/or protein in a cell (or population of cells) in comparison to a corresponding cell which has not been modified (or population of cells) by introduction of the nucleic acid molecule, construct or vector. For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has not been modified according to the present invention (or population of such cells). Preferably the cell is a Treg or the population of cells is a population of Tregs. Suitably, the level of FOXP3 mRNA and/or protein in a modified cell (or a population of such cells) may be increased to at least 1.5-fold greater, 2-fold greater, or 5-fold greater than the level in a corresponding cell which has not been so modified (or population of such cells). Preferably the cell is a Treg or the population of cells is a population of Tregs.

“Enhancing (or alternatively viewed, increasing or improving) FOXP3 expression from a nucleic acid molecule” means to increase the levels of FOXP3 mRNA and/or protein in a cell (or population of cells) (e.g. a Treg or a CD45RA+ Treg) comprising the nucleic acid molecule (an exogenous nucleic acid molecule) (or construct or vector) (i.e. a nucleic acid molecule according to the invention) in comparison to a corresponding cell (or population of cells) which has been modified by introduction of a nucleic acid molecule, construct or vector comprising (i) a nucleotide sequence encoding a safety switch polypeptide comprising a suicide moiety;
(ii) a nucleotide sequence encoding FOXP3; and
(iii) a nucleotide sequence encoding a chimeric antigen receptor (CAR), wherein (i), (ii) and (iii) are not positioned 5′ to 3′ in the order of (i), (ii) and (iii).

As previously discussed, the enhanced, increased or improved expression of FOXP3 from the nucleic acid molecule relates to the ordering of the nucleotide sequences encoding the safety switch polypeptide, the FOXP3 and the CAR within the nucleic acid molecule, and a thus a cell comprising a nucleic acid molecule of the invention has an enhanced level of FOXP3 mRNA and/or protein as compares to the same cell type comprising a nucleic acid molecule wherein the nucleotide sequences are placed within a different order within the nucleic acid molecule.

Particularly, the nucleic acid, construct or vector introduced to the comparative corresponding cell may comprise 5′ to 3′:
1) (ii) a nucleotide sequence encoding FOXP3, (i) a nucleotide sequence encoding a safety switch polypeptide comprising a suicide moiety and (iii) a nucleotide sequence encoding a CAR
2) (ii) a nucleotide sequence encoding FOXP3, (iii) a nucleotide sequence encoding a CAR and (i) a nucleotide sequence encoding a safety switch polypeptide comprising a suicide moiety
3) (i) a nucleotide sequence encoding a safety switch polypeptide comprising a suicide moiety, (iii) a nucleotide sequence encoding a CAR and (ii) a nucleotide sequence encoding FOXP3
4) (iii) a nucleotide sequence encoding a CAR, (ii) a nucleotide sequence encoding FOXP3 and (i) a nucleotide sequence encoding a safety switch polypeptide comprising a suicide moiety
5) (iii) a nucleotide sequence encoding a CAR, (i) a nucleotide sequence encoding a safety switch polypeptide comprising a suicide moiety and (ii) a nucleotide sequence encoding FOXP3

Particularly, the nucleic acid molecule, construct or vector introduced to the comparative corresponding cell may encode the same safety switch, FOXP3 and CAR polypeptides as a nucleic acid molecule, construct or vector introduced into a cell of the invention.

For example, the level of FOXP3 mRNA and/or protein in a cell modified according to the present invention (or a population of such cells) may be increased to at least 1.5-fold, at least 2-fold, at least 5-fold, at least 10-fold, at least 50-fold, at least 100-fold, at least 150-fold greater than the level in a corresponding cell which has been modified by introduction of a nucleic acid molecule, construct or vector comprising (i) a nucleotide sequence encoding a safety switch polypeptide comprising a suicide moiety;
(ii) a nucleotide sequence encoding FOXP3; and
(iii) a nucleotide sequence encoding a chimeric antigen receptor (CAR), wherein (i), (ii) and (iii) are not positioned 5′ to 3′ in the order of (i), (ii) and (iii) (or population of such cells). Preferably the cell is a Treg or the population of cells is a population of Tregs. Suitably, the level of FOXP3 mRNA and/or protein in a modified cell (or a population of such cells) may be increased to at least 1.5-fold greater, 2-fold greater, or 5-fold greater than the level in a corresponding cell which has been modified by introduction of a nucleic acid molecule, construct or vector comprising (i) a nucleotide sequence encoding a safety switch polypeptide comprising a suicide moiety;
(ii) a nucleotide sequence encoding FOXP3; and
(iii) a nucleotide sequence encoding a chimeric antigen receptor (CAR), wherein (i), (ii) and (iii) are not positioned 5′ to 3′ in the order of (i), (ii) and (iii) (or population of such cells). Preferably the cell is a Treg or the population of cells is a population of Tregs.

Techniques for measuring the levels of specific mRNA and protein are well known in the art. mRNA levels in a population of cells, such as Tregs, may be measured by techniques such as the Affymetrix ebioscience prime flow RNA assay, Northern blotting, serial analysis of gene expression (SAGE) or quantitative polymerase chain reaction (qPCR). Protein levels in a population of cells may be measured by techniques such as flow cytometry, high-performance liquid chromatography (HPLC), liquid chromatography-mass spectrometry (LC/MS), Western blotting or enzyme-linked immunosorbent assay (ELISA).

A “FOXP3 polypeptide” is a polypeptide having FOXP3 activity i.e. a polypeptide able to bind FOXP3 target DNA and function as a transcription factor regulating development and function of Tregs. Particularly, a FOXP3 polypeptide may have the same or similar activity to wildtype FOXP3 (SEQ ID NO. 2), e.g. may have at least 40, 50, 60, 70, 80, 90, 95, 100, 110, 120, 130, 140 or 150% of the activity of the wildtype FOXP3 polypeptide. Thus, a FOXP3 polypeptide encoded by the second nucleotide sequence in the nucleic acid, construct or vector described herein may have increased or decreased activity compared to wildtype FOXP3. Techniques for measuring transcription factor activity are well known in the art. For example, transcription factor DNA-binding activity may be measured by ChIP. The transcription regulatory activity of a transcription factor may be measured by quantifying the level of expression of genes which it regulates. Gene expression may be quantified by measuring the levels of mRNA and/or protein produced from the gene using techniques such as Northern blotting, SAGE, qPCR, HPLC, LC/MS, Western blotting or ELISA. Genes regulated by FOXP3 include cytokines such as IL-2, IL-4 and IFN-γ (Ziegler et al. Annu. Rev. Immunol. 2006, 24:209-26, incorporated herein by reference). As discussed in detail below, FOXP3 or a FOXP3 polypeptide includes functional fragments, variants, and isoforms thereof, e.g. of SEQ ID NO. 2.

A “functional fragment of FOXP3” may refer to a portion or region of a FOXP3 polypeptide or a polynucleotide (i.e. nucleotide sequence) encoding a FOXP3 polypeptide that has the same or similar activity to the full-length FOXP3 polypeptide or polynucleotide. The functional fragment may have at least 40%, at least 50%, at least 60%, at least 70%, at least 80%, at least 90%, at least 95%, or 100% of the activity of the full-length FOXP3 polypeptide or polynucleotide. A person skilled in the art would be able to generate functional fragments based on the known structural and functional features of FOXP3. These are described, for instance, in Song, X., et al., 2012. Cell reports, 1 (6), pp. 665-675; Lopes, J. E., et al., 2006. The Journal of Immunology, 177 (5), pp. 3133-3142; and Lozano, T., et al, 2013. Frontiers in oncology, 3, p. 294. Further, a N and C terminally truncated FOXP3 fragment is described within WO2019/241549 (incorporated herein by reference), for example, having the sequence SEQ ID NO. 6 as discussed below.

A “FOXP3 variant” may include an amino acid sequence or a nucleotide sequence which may be at least 50%, at least 55%, at least 65%, at least 70%, at least 75%, at least 80%, at least 85% or at least 90% identical, preferably at least 95% or at least 97% or at least 99% identical to a FOXP3 polypeptide or a polynucleotide encoding a FOXP3 polypeptide, e.g. to SEQ ID NO. 2. FOXP3 variants may have the same or similar activity to a wildtype FOXP3 polypeptide or polynucleotide, e.g. may have at least 40, 50, 60, 70, 80, 90, 95, 100, 110, 120, 130, 140 or 150% of the activity of a wildtype FOXP3 polypeptide or polynucleotide. A person skilled in the art would be able to generate FOXP3 variants based on the known structural and functional features of FOXP3 and/or using conservative substitutions. FOXP3 variants may have similar or the same turnover time (or degradation rate) within a Treg cell as compared to wildtype FOXP3, e.g. at least 40, 50, 60, 70, 80, 90, 95, 99 or 100% of the turnover time (or degradation rate) of wildtype FOXP3 in a Treg. Some FOXP3 variants may have a reduced turnover time (or degradation rate) as compared to wildtype FOXP3, for example, FOXP3 variants having amino acid substitutions at amino acid 418 and/or 422 of SEQ ID NO. 2, for example S418E and/or S422A, as described in WO2019/241549 (incorporated herein by reference) and are set out in SEQ ID NOs. 3 to 5, which represent the aa418, aa422 and aa418 and aa422 mutants respectively.

Suitably, the FOXP3 polypeptide encoded by a nucleic acid molecule, construct or vector as described herein may comprise or consist of the polypeptide sequence of a human FOXP3, such as UniProtKB accession Q9BZS1 (SEQ ID NO. 2), or a functional fragment or variant thereof.

In some embodiments of the invention, the FOXP3 polypeptide comprises or consists of an amino acid sequence which is at least 70% identical to SEQ ID NO. 2 or a functional fragment thereof. Suitably, the FOXP3 polypeptide comprises or consists of an amino acid sequence which is at least 80%, at least 85%, at least 90%, at least 95%, at least 98% or at least 99% identical to SEQ ID NO. 2 or a functional fragment thereof. In some embodiments, the FOXP3 polypeptide comprises or consists of SEQ ID NO. 2 or a functional fragment thereof.

In some embodiments, as discussed above, the FOXP3 polypeptide may comprise mutations at residues 418 and/or 422 of SEQ ID NO. 2, as set out in SEQ ID NO. 3, SEQ ID NO. 4, or SEQ ID NO. 5.

In some embodiments of the invention, the FOXP3 polypeptide may be truncated at the N and/or C terminal ends, resulting in the production of a functional fragment. Particularly, an N and C terminally truncated functional fragment of FOXP3 may comprise or consist of an amino acid sequence of SEQ ID NO. 6 or a functional variant thereof having at least 80, 85, 90, 95 or 99% identity thereto.

Suitably, the FOXP3 polypeptide may be a variant of SEQ ID NO. 2, for example a natural variant. Suitably, the FOXP3 polypeptide is an isoform of SEQ ID NO. 2. For example, the FOXP3 polypeptide may comprise a deletion of amino acid positions 72-106 relative to SEQ ID NO. 2. Alternatively, the FOXP3 polypeptide may comprise a deletion of amino acid positions 246-272 relative to SEQ ID NO. 2.

Suitably, the FOXP3 polypeptide comprises SEQ ID NO. 7 or a functional fragment thereof. SEQ ID NO. 7 represents an Illustrative FOXP3 polypeptide.

Suitably the FOXP3 polypeptide comprises or consists of an amino acid sequence which is at least 70% identical to SEQ ID NO. 7 or a functional fragment thereof. Suitably, the FOXP3 polypeptide comprises an amino acid sequence which is at least 80%, at least 85%, at least 90%, at least 95%, at least 98% or at least 99% identical to SEQ ID NO. 7 or a functional fragment thereof. In some embodiments, the FOXP3 polypeptide comprises or consists of SEQ ID NO. 7 or a functional fragment thereof.

Suitably, the FOXP3 polypeptide may be a variant of SEQ ID NO. 7, for example a natural variant. Suitably, the FOXP3 polypeptide is an isoform of SEQ ID NO. 7 or a functional fragment thereof. For example, the FOXP3 polypeptide may comprise a deletion of amino acid positions 72-106 relative to SEQ ID NO. 7. Alternatively, the FOXP3 polypeptide may comprise a deletion of amino acid positions 246-272 relative to SEQ ID NO. 7.

Suitably, the polynucleotide encoding a FOXP3 polypeptide comprises or consists of a nucleotide sequence set forth in SEQ ID NO. 8, which represents an illustrative FOXP3 nucleotide sequence.

In some embodiments of the invention, the polynucleotide encoding the FOXP3 polypeptide or variant comprises nucleotide sequence which is at least 70% identical to SEQ ID NO. 8 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO. 8 or a fragment thereof which encodes a functional FOXP3 polypeptide. In some embodiments of the invention, the polynucleotide encoding the FOXP3 polypeptide or variant comprises or consists of SEQ ID NO. 8 or a fragment thereof which encodes a functional FOXP3 polypeptide.

Suitably, the polynucleotide encoding a FOXP3 polypeptide comprises or consists of a polynucleotide sequence set forth in SEQ ID NO. 9, which represents another illustrative FOXP3 nucleotide.

In some embodiments of the invention, the polynucleotide encoding the FOXP3 polypeptide or variant comprises a nucleotide sequence which is at least 70% identical to SEQ ID NO. 9 or a fragment thereof which encodes a functional FOXP3 polypeptide. Suitably, the polynucleotide encoding the FOXP3 polypeptide or variant comprises a polynucleotide sequence which is at least 80%, at least 85%, at least 90%, at least 95%, at least 98% or at least 99% identical to SEQ ID NO. 9 or a fragment thereof which encodes a functional FOXP3 polypeptide. In some embodiments of the invention, the polynucleotide encoding the FOXP3 polypeptide or variant comprises or consists of SEQ ID NO. 9 or a fragment thereof which encodes a functional FOXP3 polypeptide.

Suitably, the polynucleotide encoding the FOXP3 polypeptide or functional fragment or variant thereof may be codon optimised. Suitably, the polynucleotide encoding the FOXP3 polypeptide or functional fragment or variant thereof may be codon optimised for expression in a human cell.

The third component, encoded by the third nucleotide sequence, is a CAR. The term “chimeric antigen receptor” or “CAR” as used herein refers to engineered receptors which can confer an antigen specificity onto cells (for example Tregs). CARs are also known as artificial T-cell receptors, chimeric T-cell receptors or chimeric immunoreceptors. A CAR typically comprises an extracellular domain comprising an antigen-specific targeting region, termed herein an antigen-binding domain, a transmembrane domain, and an endodomain comprising optionally one or more co-stimulatory domains, and an intracellular signaling domain. The antigen-binding domain is typically joined to the transmembrane domain by a hinge domain. The design of CARs, and the various domains that they may contain, is well known in the art.

When the CAR binds its target antigen, this results in the transmission of an activating signal to the cell in which it is expressed. Thus, the CAR directs the specificity of the engineered cells towards the target antigen, particularly towards cells expressing the targeted antigen.

The antigen-binding domain of a CAR may be derived or obtained from any protein or polypeptide which binds (i.e. has affinity for) a desired target antigen, or more generally a desired target molecule. This may be for example, a ligand or receptor, or a physiological binding protein for the target molecule, or a part thereof, or a synthetic or derivative protein. The target molecule may commonly be expressed on the surface of a cell, for example a target cell, or a cell in the vicinity of a target cell (for a bystander effect), but need not be. Depending on the nature and specificity of the antigen binding domain, the CAR may recognise a soluble molecule, for example where the antigen-binding domain is based on, or derived from, a cellular receptor.

The antigen-binding domain is most commonly derived from antibody variable chains (for example it commonly takes the form of a scFv), but may also be generated from T-cell receptor variable domains or, as mentioned above, other molecules, such as receptors for ligands or other binding molecules.

The CAR is typically expressed as a polypeptide also comprising a signal sequence (also known as a leader sequence)), and in particular a signal sequence which targets the CAR to the plasma membrane of the cell. This will generally be positioned next to or close to the antigen-binding domain, generally upstream of the antigen-binding domain. The extracellular domain, or ectodomain, of the CAR may thus comprise a signal sequence and an antigen-binding domain.

The antigen-binding domain provides the CAR with the ability to bind a predetermined antigen of interest. The antigen-binding domain preferably targets an antigen of clinical interest or an antigen at a site of disease.

As noted above, the antigen-binding domain may be any protein or peptide that possesses the ability to specifically recognize and bind to a biological molecule (e.g., a cell surface receptor or a component thereof). The antigen-binding domain includes any naturally occurring, synthetic, semi-synthetic, or recombinantly produced binding partner for a biological molecule of interest. Illustrative antigen-specific targeting domains include antibodies or antibody fragments or derivatives, extracellular domains of receptors, ligands for cell surface molecules/receptors, or receptor binding domains thereof, and tumor binding proteins. Although as discussed below, the antigen-specific targeting domain may preferably be an antibody or derived from an antibody, other antigen-specific targeting domains are encompassed, e.g. antigen-specific targeting domains formed from an antigenic peptide/MHC or HLA combination which is capable of binding to the TCRs of Tcon cells active at a site of transplantation, inflammation or disease.

In an embodiment the antigen binding domain is, or is derived from, an antibody. An antibody-derived binding domain can be a fragment of an antibody or a genetically engineered product of one or more fragments of the antibody, which fragment is involved in binding with the antigen. Examples include a variable region (Fv), a complementarity determining region (CDR), Fab or F (ab′) 2, or the light and heavy chain variable regions can be joined together in a single chain (e.g. as a ScFv) and in either orientation (e.g. VL-VH or VH-VL). The VL and/or VH sequences may be modified. In particular the framework regions may be modified (e.g, substituted, for example to humanise the antigen-binding domain). Other examples include a heavy chain variable region (VH), a light chain variable region (VL) a camelid antibody (VHH) and a single domain antibody (sAb).

In a preferred embodiment, the binding domain is a single chain antibody (scFv). The scFv may be murine, human or humanized scFv.

“Complementarity determining region” or “CDR” with regard to an antibody or antigen-binding fragment thereof refers to a highly variable loop in the variable region of the heavy chain or the light chain of an antibody. CDRs can interact with the antigen conformation and largely determine binding to the antigen (although some framework regions are known to be involved in binding). The heavy chain variable region and the light chain variable region each contain 3 CDRs. “Heavy chain variable region” or “VH” refers to the fragment of the heavy chain of an antibody that contains three CDRs interposed between flanking stretches known as framework regions, which are more highly conserved than the CDRs and form a scaffold to support the CDRs. “Light chain variable region” or “VL” refers to the fragment of the light chain of an antibody that contains three CDRs interposed between framework regions.

“Fv” refers to the smallest fragment of an antibody to bear the complete antigen binding site. An Fv fragment consists of the variable region of a single light chain bound to the variable region of a single heavy chain. “Single-chain Fv antibody” or “scFv” refers to an engineered antibody consisting of a light chain variable region and a heavy chain variable region connected to one another, in either orientation, directly or via a peptide linker sequence.

Antibodies that specifically bind a predetermined antigen can be prepared using methods well known in the art. Such methods include phage display, methods to generate human or humanized antibodies, or methods using a transgenic animal or plant engineered to produce human antibodies. Phage display libraries of partially or fully synthetic antibodies are available and can be screened for an antibody or fragment thereof that can bind to the target molecule. Phage display libraries of human antibodies are also available. Once identified, the amino acid sequence or polynucleotide sequence coding for the antibody can be isolated and/or determined.

The CAR may be directed towards any desired target antigen or molecule. This may be selected according to the intended therapy, and the condition it is desired to treat. It may for example be an antigen or molecule associated with a particular condition, or an antigen or molecule associated with a cell it is desired to target to treat the condition. Typically, the antigen or molecule is a cell-surface antigen or molecule.

The term “directed against” is synonymous with “specific for” or “anti”. Put another way, the CAR recognises a target molecule. Accordingly, it is meant that the CAR is capable of binding specifically to a specified or given antigen, or target. In particular, the antigen-binding domain of the CAR is capable of binding specifically to the target molecule or antigen (more particularly when the CAR is expressed on the surface of a cell, notably an immune effector cell). Specific binding may be distinguished from non-specific binding to a non-target molecule or antigen. Thus, a cell expressing the CAR is directed, or re-directed, to bind specifically to a target cell, expressing the target molecule or antigen, particularly a target cell expressing the target antigen or molecule on its cell surface.

Antigens which may be targeted by the present CAR include, but are not limited to, antigens expressed on cells associated with transplanted organs, autoimmune diseases, allergic diseases and inflammatory diseases (e.g. neurodegenerative disease). It will be understood by a skilled person that where the cell engineered to express the nucleic acid molecule is a Treg cell, or a precursor therefor, due to the bystander effect of Treg cells, the antigen may be simply present and/or expressed at the site of transplantation, inflammation or disease.

Antigens expressed on cells associated with neurodegenerative disease include those presented on glial cells, e.g. MOG.

Antigens associated with organ transplants and/or cells associated with transplanted organs include, but are not limited to, a HLA antigen present in the transplanted organ but not in the patient, or an antigen whose expression is up-regulated during transplant rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

In an embodiment the CAR is directed against an HLA antigen, and in particular an HLA-A2 antigen.

In an embodiment, the CAR is not directed against MHC II.

Antibodies against such antigens and are known in the art, and conveniently a scFv may be obtained or generated bases on a known or available antibody. In this regard VH and VL, and CDR sequences are publically available to aid the preparation of such an antibody-binding domain, for example in WO 2020/044055, the disclosure of which is herein incorporated by reference. Any of the antigen binding domains, or CDR, VH, and/or VL sequences disclosed in WO 2020/044055 may be used.

By way of example, the CAR may comprise an antigen binding domain which is capable of binding HLA-A2 (HLA-A2 may also be referred to herein as HLA-A*02, HLA-A02, and HLA-A*2). HLA-A*02 is one particular class I major histocompatibility complex (MHC) allele group at the HLA-A locus.

The antigen recognition domain may bind, suitably specifically bind, one or more regions or epitopes within HLA-A2. An epitope, also known as antigenic determinant, is the part of an antigen that is recognised by an antigen recognition domain (e.g. an antibody). In other words, the epitope is the specific piece of the antigen to which an antibody binds. Suitably, the antigen recognition domain binds, suitably specifically binds, to one region or epitope within HLA-A2.

The antigen recognition domain may comprise at least one CDR (e.g. CDR3), which can be predicted from an antibody which binds to an antigen, e.g. HLA-A2 (or a variant of such a predicted CDR (e.g. a variant with one, two or three amino acid substitutions)). It will be appreciated that molecules containing three or fewer CDR regions (e.g. a single CDR or even a part thereof) may be capable of retaining the antigen-binding activity of the antibody from which the CDR is derived. Molecules containing two CDR regions are described in the art as being capable of binding to a target antigen, e.g. in the form of a minibody (Vaughan and Sollazzo, 2001, Combinational Chemistry & High Throughput Screening, 4, 417-430). Molecules containing a single CDR have been described which can display strong binding activity to target (Nicaise et al, 2004, Protein Science, 13:1882-91).

In this respect, the antigen binding domain may comprise one or more variable heavy chain CDRs, e.g. one, two or three variable heavy chain CDRs. Alternatively, or additionally, the antigen binding domain may comprise one or more variable light chain CDRs, e.g. one, two or three variable light chain CDRs. The antigen binding domain may comprise three heavy chain CDRs and/or three light chain CDRs (and more particularly a heavy chain variable region comprising three CDRs and/or a light chain variable region comprising three CDRs) wherein at least one CDR, preferably all CDRs, may be from an antibody which binds to an antigen, e.g. HLA-A2, or may be selected from one of the CDR sequences disclosed in WO 2020/044055.

The antigen binding domain may comprise any combination of variable heavy and light chain CDRs, e.g. one variable heavy chain CDR together with one variable light chain CDR, two variable heavy chain CDRs together with one variable light chain CDR, two variable heavy chain CDRs together with two variable light chain CDRs, three variable heavy chain CDRs together with one or two variable light chain CDRs, one variable heavy chain CDR together with two or three variable light chain CDRs, or three variable heavy chain CDRs together with three variable light chain CDRs. Preferably, the antigen binding domain comprises three variable heavy chain CDRs (CDR1, CDR2 and CDR3) and/or three variable light chain CDRs (CDR1, CDR2 and CDR3).

The one or more CDRs present within the antigen binding domain may not all be from the same antibody, as long as the domain has the desired binding activity. Thus, one CDR may be predicted from the heavy or light chains of an antibody which binds to an antigen, e.g. HLA-A2 whilst another CDR present may be predicted from a different antibody which binds to the same antigen (e.g. HLA-A2). In this instance, it may be preferred that CDR3 be predicted from an antibody that binds to an antigen, e.g. HLA-A2. Particularly however, if more than one CDR is present in the antigen binding domain, it is preferred that the CDRs are predicted from antibodies which bind to the same antigen, e.g. HLA-A2. A combination of CDRs may be used from different antibodies, particularly from antibodies that bind to the same desired region or epitope.

In a particularly preferred embodiment, the antigen binding domain comprises three CDRs predicted from the variable heavy chain sequence of an antibody which binds to an antigen, e.g. HLA-A2 and/or three CDRs predicted from the variable light chain sequence of an antibody which binds to an antigen, e.g. HLA-A2 (preferably the same antibody).

In an embodiment, the antigen-binding domain comprises VH CDR1, 2 and 3 sequences as set forth in SEQ ID NOs. 11, 12 and 13 respectively and VL CDR1, 2 and 3 sequences as set forth in SEQ ID NOs. 14, 15 and 16 respectively, or the CDRs may contain 1 to 3, or more particularly 1 or 2 amino acid sequence modifications to the CDR sequences set out in any one or more of SEQ ID NO. 11, 12, 13, 14, 15, or 16.

More particularly, in such an embodiment the antigen binding domain of the CAR comprises a VH domain comprising the sequence as set forth in SEQ ID NO. 17, or a sequence having at least 70% sequence identity thereto, and a VL domain comprising the sequence as set forth in SEQ ID NO. 18, or a sequence having at least 70% sequence identity thereto.

In another embodiment, the antigen-binding domain comprises VH CDR1, 2 and 3 sequences as set forth in SEQ ID NOs. 20, 21 and 22 respectively and VL CDR1, 2 and 3 sequences as set forth in SEQ ID NOs. 23, 24, and 25 respectively, or the CDRs may contain 1 to 3, or more particularly 1 or 2 amino acid sequence modifications to the CDR sequences set out in any one or more of SEQ ID NO. 20, 21, 22, 23, 24, or 25.

Where a CDR does contain an amino acid sequence modification, this may be a deletion, addition, or substitution of an amino acid residue of the CDR sequence as set out in the above-mentioned SEQ ID NOs. More particularly, the modification may be an amino acid substitution, for example a conservative amino acid substitution, e.g. as set out above. A longer CDR may tolerate more amino acid residue modifications. In the case of CDRs which are 5 or 7 amino acid residues long, the modifications may be to or of 1 or 2, e.g. 1, residue. In general, there may be 0, 1, 2, or 3 modifications to any particular CDR sequence. Further, in an embodiment, CDRs 1 and 2 may be modified, and CDR3 may be unmodified. In another embodiment all 3 CDRs may be modified. In another embodiment, the CDRs are not modified.

More particularly, in such an embodiment the antigen binding domain of the CAR comprises a VH domain comprising the sequence as set forth in SEQ ID NO. 26, or a sequence having at least 70% sequence identity thereto, and a VL domain comprising the sequence as set forth in SEQ ID NO. 27, or a sequence having at least 70% sequence identity thereto.

The antigen binding domain may be in the form of a scFV comprising the VH and VL domain sequences as set out above, in either order, for example VH-VL. The VH and VL sequences may be linked by a linker sequence.

Suitable linkers can be readily selected and can be of any of a suitable length, such as from 1 amino acid (e.g. Gly) to 30 amino acids (SEQ ID NO: 98), e.g. from any one of 2, 3, 4, 5, 6, 7, 8, 9, or 10 amino acids to any one of 12, 15, 18, 20, 21, 25, 30 amino acids, for example, 5-30, 5-25, 6-25, 10-15, 12-25, 15 to 25 etc.

The linker may for example be a linker as discussed above in relation to the safety switch polypeptide. Exemplary flexible linkers include glycine polymers (G), glycine-serine polymers, where n is an integer of at least one, glycine-alanine polymers, alanine-serine polymers, and other flexible linkers known in the art, as discussed above. The linker may comprise 1 or more “GS” domains as discussed above.

Accordingly in one embodiment the antigen binding domain may comprise, or consist of, a sequence as set forth in SEQ ID NO. 19 which comprises the VH sequence of SEQ ID NO. 17 linked via a linker of sequence (X)n, where X is any amino acid and n is an integer of between 15 and 25, to the VL sequence of SEQ ID NO. 18. In a particular embodiment the antigen binding domain comprises, or consists of, a sequence as set forth in SEQ ID NO. 88 which corresponds to a sequence of SEQ ID NO. 19, wherein the linker has the sequence of SEQ ID NO. 89 (LVTVSSGGGGSGGGGSGGGGST). The antigen binding domain may comprise or consist of a sequence which is a variant of SEQ ID NO. 19 or 88 which has at least 70% sequence identity thereto.

In another embodiment the antigen binding domain may comprise a sequence as set forth in SEQ ID NO. 28, or a variant thereof having a sequence with at least 70% sequence identity thereto.

The variant sequences disclosed and described herein, including the variant VH, VL and antigen binding domain sequences, may have at least 75, 80, 85, 90, 92, 95, 96, 97, 98, or 99% sequence identity to the specified SEQ ID NOs.

The CAR also preferably comprises a hinge domain to hold the extracellular domain, particularly the antigen binding domain, away from the cell surface, and comprises a transmembrane domain. The hinge and transmembrane domains may comprise the hinge and transmembrane sequences from any protein which has a hinge domain and/or a transmembrane domain, including any of the type I, type II or type Ill transmembrane proteins. Typically, the hinge may be derived from CD8, particularly, CD8alpha.

The transmembrane domain of the CAR may also comprise an artificial hydrophobic sequence. The transmembrane domains of the CAR may be selected so as not to dimerize. Additional transmembrane domains will be apparent to those of skill in the art. Examples of transmembrane (TM) regions used in CAR constructs are: 1) The CD28 TM region (Pule et al, Mol Ther, 2005 November; 12 (5): 933-41; Brentjens et al, CCR, 2007 September 15; 13 (18 Pt 1): 5426-35; Casucci et al, Blood, 2013 November 14; 122 (20): 3461-72.); 2) The OX40 TM region (Pule et al, Mol Ther, 2005 November; 12 (5): 933-41); 3) The 41BB TM region (Brentjens et al, CCR, 2007 September 15; 13 (18 Pt 1): 5426-35); 4). The CD3 zeta TM region (Pule et al, Mol Ther, 2005 November; 12 (5): 933-41; Di Stasi et al, Blood, 2009 Jun. 18; 113 (25): 6392-402.); 5) The CD8a TM region (Maher et al, Nat Biotechnol, 2002 January; 20 (1): 70-5; Imai C, Leukemia, 2004 April; 18 (4): 676-84; Brentjens et al, CCR, 2007 Sep. 15; 13 (18 Pt 1): 5426-35; Milone et al, Mol Ther, 2009 August; 17 (8): 1453-64). Other transmembrane domains which may be used include those from CD4, CD45, CD9, CD16, CD22, CD33, CD64, CD80, CD86, or CD154.

A hinge domain may conveniently be obtained from the same protein as the transmembrane domain, although this is not essential.

By way of example the transmembrane domain may be derived from the CD28 transmembrane domain, and may comprise the amino acid sequence shown as SEQ ID NO. 73, or a variant which is at least 80% identical to SEQ ID NO. 73 The variant may be at least 80, 85, 90, 95, 97, 98 or 99% identical to SEQ ID NO. 73.

Alternatively, the CAR may comprise a domain derived from the CD8a transmembrane domain. Thus, the transmembrane domain may comprise the amino acid sequence shown as SEQ ID NO. 67 which represents amino acids 183 to 203 of human CD8α, or a variant which is at least 80% identical to SEQ ID NO. 67. Suitably, the variant may be at least 85, 90, 95, 97, 98 or 99% identical to SEQ ID NO. 67.

The CD8α transmembrane domain may be combined with a CD8α hinge domain. In an embodiment the CAR comprises a combined CD8α hinge and transmembrane domain sequence as shown in SEQ ID NO. 68, or a variant thereof which has at least 80% sequence identity thereto. The variant may be at least 85, 90, 95, 97, 98 or 99% identical to SEQ ID NO. 68. SEQ ID NO. 68 comprises a modified hinge domain comprising 2 amino acid substitutions of cysteine residues relative to the wild-type CD8α hinge sequence. The modified CD8α hinge domain sequence is shown in SEQ ID NO. 69. The wildtype CD8α hinge domain sequence is shown in SEQ ID NO. 70. A variant of such hinge sequences having at least 80% sequence identity to SEQ ID NO. 69 or 70 may be used.

An example of a CD28 hinge and transmembrane sequence which may be used is SEQ ID NO. 74 or a variant thereof which is at least 80% identical to SEQ ID NO. 74. The variant may be at least 85, 90, 95, 97, 98 or 99% identical to SEQ ID NO. 74.

By way of further example, the CAR may comprise a native or modified CD8α hinge domain and a CD28 transmembrane domain, or a CD28 hinge domain and CD8α transmembrane domain, for example based on the sequences given above.

Other hinge domains which may be used include those from CD4, CD7, or an immunoglobulin, or a part or variant thereof.

The CAR may further comprise a signal (or alternatively termed, leader) sequence which targets it to the endoplasmic reticulum pathway for expression on the cell surface. An illustrative signal/leader sequence is MALPVTALLLPLALLLHAAAP as shown in SEQ ID NO. 66. This comprises a single amino acid substitution compared to the wild type CD8α sequence MALPVTALLLPLALLLHAARP as shown in SEQ ID NO. 75. Either sequence, or a variant sequence having at least 70% sequence identity thereto may be used.

The endodomain of a CAR as described herein comprises motifs necessary to transduce the effector function signal and direct a cell expressing the CAR to perform its specialized function upon antigen binding. Particularly, the endodomain may comprise one or more (e.g. two or three) Immunoreceptor tyrosine-based activation motifs (ITAMs), typically comprising the amino acid sequence of YXXL/I, where X can be any amino acid. Examples of intracellular signaling domains include, but are not limited to, ζ chain endodomain of the T-cell receptor or any of its homologs (e.g., n chain, FcεR1γ and β chains, MB1 (Igα) chain, B29 (Igβ) chain, etc.), CD3 polypeptide domains (Δ, δ and ε), syk family tyrosine kinases (Syk, ZAP 70, etc.), src family tyrosine kinases (Lck, Fyn, Lyn, etc.) and other molecules involved in T-cell transduction, such as CD2, CD5 and CD28. The intracellular signaling domain may comprise human CD3 zeta chain endodomain, FcyRIII, FcsRI, cytoplasmic tails of Fc receptors, immunoreceptor tyrosine-based activation motif (ITAM) bearing cytoplasmic receptors or combinations thereof.

Commonly, the intracellular signaling domain comprises the intracellular signaling domain of a human CD3 zeta chain. The sequence of the intracellular signaling domain of human CD3 zeta chain is set out in SEQ ID NO. 76. A modified human CD3ζ intracellular domain sequence comprising an amino acid deletion relative to the wild-type is shown in SEQ ID NO. 72. The CAR may comprise a CD3ζ signalling domain comprising or consisting of a sequence as set out in SEQ ID NO. 72 or 76 or a sequence having at least 80, 85, 90, 95, 97, 98 or 99% identity to SEQ ID NO. 72 or 76. In an embodiment the signaling domain comprises or consists of SEQ ID NO. 72.

Other signaling domains which may be used include the signaling domains of CD28 or CD27 or variants thereof. Additional intracellular signaling domains will be apparent to those of skill in the art and may be used in connection with alternate embodiments of the invention. In one embodiment, the present CAR may not comprise a costimulatory domain derived from 41BB within the endodomain.

The present CAR may comprise a compound endodomain comprising a fusion of the intracellular part of a T-cell co-stimulatory molecule to that of e.g. CD3Z. Such a compound endodomain may be referred to as a second-generation CAR which can transmit an activating and co-stimulatory signal simultaneously after antigen recognition. The co-stimulatory domain most commonly used is that of CD28. This supplies the most potent co-stimulatory signal-namely immunological signal 2, which triggers T-cell proliferation. The CAR endodomain may also comprise one or more TNF receptor family signalling domain, such as the signalling domain of OX40, 4-1BB, ICOS or TNFRSF25, although preferably the CAR may not comprise an endodomain comprising the signalling domains of both CD28 and 41BB.

The intracellular signaling domain of CD28 which may be used as a co-stimulatory domain is shown in SEQ ID NO. 77. A variant CD28 co-stimulatory domain comprising an additional 2 amino acids (WV) at the N-terminus, derived from the transmembrane domain of CD28, is shown in SEQ ID NO. 71. Illustrative sequences for OX40, 4-1BB, ICOS and TNFRSF25 signalling domains which are shown in SEQ ID NO. 79 to 81 respectively. The CAR may comprise one or more co-stimulatory domains comprising or consisting the sequence of any one of SEQ ID NO. 71, 77, or 79 to 81 or a variant thereof having at least 80, 85, 90, 95, 97, 98 or 99% sequence identity thereto.

In an embodiment the CAR comprises a co-stimulatory domain comprising or consisting of SEQ ID NO. 71.

In one embodiment the CAR comprises a human CD8 hinge domain or a variant thereof and a human CD8 transmembrane domain. Alternatively, or additionally, the CAR comprises an endodomain comprising a human CD28 co-stimulatory domain and a human CD34 signalling domain.

In one preferred embodiment the CAR comprises a hinge, transmembrane, and intracellular (or endo) domains as follows:
(i) a CD8α hinge and transmembrane domain sequence comprising or consisting of the sequence as set forth in SEQ ID NO. 68, or a sequence having at least 80% sequence identity thereto;
(ii) a CD28 co-stimulatory domain comprising or consisting of the sequence as set forth in SEQ ID NO. 71, or a sequence having at least 80% sequence identity thereto;
(iii) a CD3ζ signalling domain comprising or consisting of the sequence as set forth in SEQ ID NO. 72, or a sequence having at least 80% sequence identity thereto.

The CAR, as encoded and expressed, may further comprise a leader sequence comprising or consisting of a sequence as set out in SEQ ID NO. 66, or a sequence having at least 80% sequence identity thereto.

The antigen binding domain of the CAR may comprise or consist of a sequence as set out in SEQ ID NO. 19, 88 or 28, or a sequence having at least 80% sequence identity thereto which is capable of binding to HLA-A2.

Thus, in its entirety one preferred representative CAR may comprise;
(i) a leader sequence comprising or consisting of a sequence as set out in SEQ ID NO. 66, or a sequence having at least 80% sequence identity thereto;
(ii) an antigen binding domain comprising or consisting of a sequence as set out in SEQ ID NO. 19 or 88, or a sequence having at least 80% sequence identity thereto;
(iv) a CD8α hinge and transmembrane domain sequence comprising or consisting of the sequence as set forth in SEQ ID NO. 68, or a sequence having at least 80% sequence identity thereto;
(v) a CD28 co-stimulatory domain comprising or consisting of the sequence as set forth in SEQ ID NO. 71, or a sequence having at least 80% sequence identity thereto;
(vi) a CD3ζ signalling domain comprising or consisting of the sequence as set forth in SEQ ID NO. 72, or a sequence having at least 80% sequence identity thereto. The CAR should be capable of binding to HLA-A2 and of transducing a signal into a cell in which it is expressed.

The endodomain of a CAR herein may contain further domains. For example, it may comprise a domain comprising a STAT5 association motif, a JAK1 and/or JAK 2 binding motif and optionally a JAK 3 binding motif. In such an embodiment the endodomain may comprise one or more sequences from an endodomain of a cytokine receptor, for example an interleukin receptor (IL) receptor. Such CARs are described in WO 2020/044055 (also incorporated herein by reference). The inclusion of such domains confers the ability on the CAR to provide a productive IL signal to the cell in which it is expressed in an antigen-specific manner, without requiring exogenous IL to be administered. For example, IL-2 is important for the survival, proliferation and persistence of Treg cells, but IL2 levels may frequently be low or impaired in patients needing treatment. A CAR may thus comprise a sequence corresponding to all or part of a β chain endodomain of an IL receptor or a variant thereof, such as the IL2 receptor, optionally in combination with the γ chain endodomain of an IL receptor or a variant thereof, such as the IL2 receptor.

By way of example, the CAR endodomain may comprise a domain comprising a sequence from the human IL-2 receptor β chain, or a variant thereof, as follows:
a sequence as set forth in, SEQ ID NO. 84, which represents amino acid numbers 266 to 551 of human IL-2 receptor β chain (NCBI REFSEQ: NP_000869.1), or a sequence with at least 80% sequence identity to SEQ ID NO. 84; or
a sequence as set forth in SEQ ID NO. 85, which represents a truncated and sequence modified variant of SEQ ID NO. 84 (substitution Y510), or a sequence with at least 80% sequence identity to SEQ ID NO. 85; or
a sequence as set forth in SEQ ID NO. 86, which represents a truncated and sequence modified variant of SEQ ID NO. 84 (substitutions Y510 and Y392), or a sequence with at least 80% sequence identity to SEQ ID NO. 86.

The nucleic acid molecule may comprise nucleotide sequences encoding self-cleavage sequences in between the three encoded polypeptides. Particularly, the self-cleaving sequences are self-cleaving peptides. Such sequences auto-cleave during protein production. Self-cleaving peptides which may be used are 2A peptides or 2A-like peptides which are known and described in the art, for example in Donnelly et al., Journal of General Virology, 2001, 82, 1027-1041, herein incorporated by reference. As noted above, 2A and 2A-like peptides are believed to cause ribosome skipping, and result in a form of cleavage in which a ribosome skips the formation of peptide bond between the end of a 2A peptide and the downstream amino acid sequence. The “cleavage” occurs between the Glycine and Proline residues at the C-terminus of the 2A peptide meaning the upstream cistron will have a few additional residues added to the end, while the downstream cistron will start with the Proline.

Suitable self-cleaving domains include P2A, T2A, E2A, and F2A sequences as shown in SEQ ID NO. 29-32 respectively. The sequences may be modified to include the amino acids GSG at the N-terminus of the 2A peptides. Thus, also included as possible options are sequences corresponding to SEQ ID NOs. 29-32, but with GSG at the N termini thereof. Such modified alternative 2A sequences are known and reported in the art. Alternative 2A-like sequences which may be used are shown in Donnelly et al (supra), for example a TaV sequence.

The self-cleaving sequences included in the nucleic acid molecule may be the same or different. In an embodiment they are both 2A sequences, in particular P2A and/or T2A sequences. In an embodiment the self-cleaving sequence between the safety switch polypeptide and FOXP3 is FOXP3 is a P2A sequence and the self-cleaving sequence between FOXP3 and the CAR is a T2A sequence.

The self-cleaving sequence may include an additional cleavage site, which may be cleaved by common enzymes present in the cell. This may assist in achieving complete removal of the 2A sequences after translation. Such an additional cleavage site may for example comprise a Furin cleavage site RXXR (SEQ ID NO. 82), for example RRKR (SEQ ID NO. 83).

In a representative embodiment, the nucleic acid molecule comprises 5′ to 3′:
(i) a first nucleotide sequence encoding a safety switch polypeptide comprising the sequence of SEQ ID NO. 10, or a sequence with at least 80% sequence identity thereto;
(ii) a nucleotide sequence encoding a P2A cleavage sequence having the sequence of SEQ ID NO. 29;
(iii) a second nucleotide sequence encoding a FOXP3 polypeptide comprising the sequence of SEQ ID NO. 2, or a sequence with at least 70% sequence identity thereto;
(iv) a nucleotide sequence encoding a T2A cleavage sequence having the sequence of SEQ ID NO. 30; and
(vi) a third nucleotide sequence encoding a CAR directed against HLA-A2.

In such an embodiment, the CAR may comprise:
(a) a leader sequence comprising or consisting of a sequence as set out in SEQ ID NO. 66, or a sequence having at least 80% sequence identity thereto;
(b) an antigen binding domain comprising or consisting of a sequence as set out in SEQ ID NO. 19 or 88, or a sequence having at least 80% sequence identity thereto;
(c) a CD8α hinge and transmembrane domain sequence comprising or consisting of the sequence as set forth in SEQ ID NO. 68, or a sequence having at least 80% sequence identity thereto;
(d) a C28 co-stimulatory domain comprising or consisting of the sequence as set forth in SEQ ID NO. 71, or a sequence having at least 80% sequence identity thereto;
(e) a CD3ζ signalling domain comprising or consisting of the sequence as set forth in SEQ ID NO. 72, or a sequence having at least 80% sequence identity thereto.

As is clear from the above description in addition to the specific polypeptide and nucleotide sequences mentioned herein, also encompassed is the use of variants, or derivatives and fragments thereof.

The term “derivative” or “variant” as used interchangeably herein, in relation to proteins or polypeptides of the present invention includes any substitution of, variation of, modification of, replacement of, deletion of and/or addition of one (or more) amino acid residues from or to the sequence providing that the resultant protein or polypeptide retains the desired function (for example, where the derivative or variant is an antigen binding domain, the desired function may be the ability of the antigen binding domain to bind its target antigen (for example, a variant of an antigen binding domain which binds to HLA-A2 retains the ability to bind HLA-A2), where the derivative or variant is a signalling domain, the desired function may be the ability of that domain to signal (e.g. activate or inactivate a downstream molecule), where the derivative or variant is a transcription factor (e.g. FOXP3), the desired function may be the ability of the transcription factor to bind to target DNA and/or to induce transcription or where the derivative or variant is a safety switch polypeptide, the desired function may be the ability of that polypeptide to induce cell death e.g. upon binding of a molecule thereto. Alternatively viewed, the variants or derivatives referred to herein are functional variants or derivatives. For example, variant or derivative may have at least at least 10%, at least 20%, at least 30%, at least 40%, at least 50%, at least 60%, at least 70%, at least 80%, or at least 90% function compared to the corresponding, reference sequence. The variant or derivative may have a similar or the same level of function as compared to the corresponding, reference sequence or may have an increased level of function (e.g. increased by at least 10%, at least 20%, at least 30%, at least 40% or at least 50%).

Typically, amino acid substitutions may be made, for example from 1, 2 or 3 to 10 or 20 substitutions provided that the modified sequence retains the required activity or ability. Amino acid substitutions may include the use of non-naturally occurring analogues. For example, the variant or derivative may have at least 10%, at least 20%, at least 30%, at least 40%, at least 50%, at least 60%, at least 70%, at least 80%, or at least 90% activity or ability compared to the corresponding, reference sequence. The variant or derivative may have a similar or the same level of activity or ability as compared to the corresponding, reference sequence or may have an increased level of activity or ability (e.g. increased by at least 10%, at least 20%, at least 30%, at least 40% or at least 50%).

Proteins or peptides may also have deletions, insertions or substitutions of amino acid residues which produce a silent change and result in a functionally equivalent protein. Deliberate amino acid substitutions may be made on the basis of similarity in polarity, charge, solubility, hydrophobicity, hydrophilicity and/or the amphipathic nature of the residues as long as the endogenous function is retained. For example, negatively charged amino acids include aspartic acid and glutamic acid; positively charged amino acids include lysine and arginine; and amino acids with uncharged polar head groups having similar hydrophilicity values include asparagine, glutamine, serine, threonine and tyrosine.

Conservative substitutions may be made, for example according to Table 2 above.

The derivative may be a homologue. The term “homologue” as used herein means an entity having a certain homology with the wild type amino acid sequence and the wild type nucleotide sequence. The term “homology” can be equated with “identity”.

A homologous or variant sequence may include an amino acid sequence which may be at least 70%, 75%, 85% or 90% identical, preferably at least 95%, 96%, 97%, 98% or 99% identical to the subject sequence. Typically, the variants will comprise the same active sites etc. as the subject amino acid sequence. Although homology can also be considered in terms of similarity (i.e. amino acid residues having similar chemical properties/functions), in the context herein it is preferred to express homology in terms of sequence identity.

Homology comparisons can be conducted by eye or, more usually, with the aid of readily available sequence comparison programs. These commercially available computer programs can calculate percentage homology or identity between two or more sequences.

Percentage homology or sequence identity may be calculated over contiguous sequences, i.e. one sequence is aligned with the other sequence and each amino acid in one sequence is directly compared with the corresponding amino acid in the other sequence, one residue at a time. This is called an “ungapped” alignment. Typically, such ungapped alignments are performed only over a relatively short number of residues.

Although this is a very simple and consistent method, it fails to take into consideration that, for example, in an otherwise identical pair of sequences, one insertion or deletion in the nucleotide sequence may cause the following codons to be put out of alignment, thus potentially resulting in a large reduction in percent homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local homology.

However, these more complex methods assign “gap penalties” to each gap that occurs in the alignment so that, for the same number of identical amino acids, a sequence alignment with as few gaps as possible, reflecting higher relatedness between the two compared sequences, will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, it is preferred to use the default values when using such software for sequence comparisons. For example, when using the GCG Wisconsin Bestfit package the default gap penalty for amino acid sequences is −12 for a gap and −4 for each extension.

Calculation of maximum percentage homology/sequence identity therefore firstly requires the production of an optimal alignment, taking into consideration gap penalties. A suitable computer program for carrying out such an alignment is the GCG Wisconsin Bestfit package (University of Wisconsin, U.S.A.; Devereux et al. (1984) Nucleic Acids Res. 12:387). Examples of other software that can perform sequence comparisons include, but are not limited to, the BLAST package (see Ausubel et al. (1999) ibid—Ch. 18), FASTA (Atschul et al. (1990) J. Mol. Biol. 403-410) and the GENEWORKS suite of comparison tools. Both BLAST and FASTA are available for offline and online searching (see Ausubel et al. (1999) ibid, pages 7-58 to 7-60). However, for some applications, it is preferred to use the GCG Bestfit program. Another tool, called BLAST 2 Sequences is also available for comparing protein and nucleotide sequences (see FEMS Microbiol. Lett. (1999) 174:247-50; FEMS Microbiol. Lett. (1999) 177:187-8).

Although the final percentage homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see the user manual for further details). For some applications, it is preferred to use the public default values for the GCG package, or in the case of other software, the default matrix, such as BLOSUM62. Suitably, the percentage identity is determined across the entirety of the reference and/or the query sequence. Once the software has produced an optimal alignment, it is possible to calculate percentage homology, preferably percentage sequence identity. The software typically does this as part of the sequence comparison and generates a numerical result.

“Fragment” typically refers to a selected region of the polypeptide or polynucleotide that is of interest functionally, e.g. is functional or encodes a functional fragment. “Fragment” thus refers to an amino acid or nucleic acid sequence that is a portion (or part) of a full-length polypeptide or polynucleotide.

Such variants, derivatives and fragments may be prepared using standard recombinant DNA techniques such as site-directed mutagenesis. Where insertions are to be made, synthetic DNA encoding the insertion together with 5′ and 3′ flanking regions corresponding to the naturally-occurring sequence either side of the insertion site may be made. The flanking regions will contain convenient restriction sites corresponding to sites in the naturally-occurring sequence so that the sequence may be cut with the appropriate enzyme(s) and the synthetic DNA ligated into the cut. The DNA is then expressed in accordance with the invention to make the encoded protein. These methods are only illustrative of the numerous standard techniques known in the art for manipulation of DNA sequences and other known techniques may also be used.

Nucleic acid molecules and polynucleotides/nucleic acid sequences as defined herein may comprise DNA or RNA. They may be single-stranded or double-stranded. It will be understood by a skilled person that numerous different nucleic acid molecules/polynucleotides can encode the same polypeptide as a result of the degeneracy of the genetic code. In addition, it is to be understood that the skilled person may, using routine techniques, make nucleotide substitutions that do not affect the polypeptide sequence encoded by the nucleic acid molecules/polynucleotides/nucleotide sequences as defined herein to reflect the codon usage of any particular host organism in which the polypeptides of the invention are to be expressed.

The nucleic acid molecules/polynucleotides may be modified by any method available in the art. Such modifications may be carried out in order to enhance the in vivo activity or lifespan of the nucleic acid molecules/polynucleotides as defined herein.

Nucleic acid molecules/polynucleotides/nucleotide sequences such as DNA nucleic acid molecules/polynucleotides/sequences may be produced recombinantly, synthetically or by any means available to those of skill in the art. They may also be cloned by standard techniques.

Longer nucleic acid molecules/polynucleotides/nucleotide sequences will generally be produced using recombinant means, for example using polymerase chain reaction (PCR) cloning techniques. This will involve making a pair of primers (e.g. of about 15 to 30 nucleotides) flanking the target sequence which it is desired to clone, bringing the primers into contact with mRNA or cDNA obtained from an animal or human cell, performing a polymerase chain reaction under conditions which bring about amplification of the desired region, isolating the amplified fragment (e.g. by purifying the reaction mixture with an agarose gel) and recovering the amplified DNA. The primers may be designed to contain suitable restriction enzyme recognition sites so that the amplified DNA can be cloned into a suitable vector.

The present nucleic acid molecule/polynucleotide may further comprise a nucleic acid sequence encoding a selectable marker. Suitably selectable markers are well known in the art and include, but are not limited to, fluorescent proteins-such as GFP. Suitably, the selectable marker may be a fluorescent protein, for example GFP, YFP, RFP, tdTomato, dsRed, or variants thereof. In some embodiments the fluorescent protein is GFP or a GFP variant. The nucleic acid sequence encoding a selectable marker may be provided in combination with a nucleic acid molecule herein in the form of a nucleic acid construct. Such a nucleic acid construct may be provided in a vector.

Suitably, the selectable marker/reporter domain may be a luciferase-based reporter, a PET reporter (e.g. Sodium Iodide Symporter (NIS)), or a membrane protein (e.g. CD34, low-affinity nerve growth factor receptor (LNGFR)).

The nucleic acid sequences encoding one or more selectable markers may be separated from the present nucleic acid molecule, and/or from each other, by one or more co-expression sites which enables expression of each polypeptide as a discrete entity. Suitable co-expression sites are known in the art and include, for example, internal ribosome entry sites (IRES) and self-cleaving sites such as those included in the present nucleic acid molecules, and as defined above. In an embodiment this may be a 2A cleavage sites, as discussed above.

The use of a selectable marker is advantageous as it allows cells (e.g. Tregs) in which a nucleic acid molecule, construct or vector of the present invention has been successfully introduced (such that the encoded safety switch polypeptide, FOXP3 and CAR are expressed) to be selected and isolated from a starting cell population using common methods, e.g. flow cytometry.

The nucleic acid molecule/polynucleotides used in the present invention may be codon-optimised. Codon optimisation has previously been described in WO 1999/41397 and WO 2001/79518. Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available.

The first nucleotide sequence, the second nucleotide sequence and the third nucleotide sequence may be provided in construct in which they are operably linked to the same promoter. A “promoter” is a region of DNA that leads to initiation of transcription of a gene. Promoters are located near the transcription start sites of genes, upstream on the DNA (towards the 5′ region of the sense strand). Any suitable promoter may be used, the selection of which may be readily made by the skilled person. The promoter may be from any source, and may be a viral promoter, or a eukaryotic promoter, including mammalian or human promoters (i.e. a physiological promoter). In an embodiment the promoter is a viral promoter.

Particular promoters include LTR promoters, EFS (or functional truncations thereof), SFFV, PGK, and CMV. In an embodiment the promoter is SFFV or a viral LTR promoter. Particularly, a SFFV promoter may be used within a nucleic acid molecule, construct or vector of the invention to allow initiation of transcription of (i) the nucleotide sequence encoding the safety switch polypeptide comprising a suicide moiety;
(ii) the nucleotide sequence encoding FOXP3; and
(iii) the nucleotide sequence encoding a chimeric antigen receptor (CAR). Thus, alternatively viewed, (i) the nucleotide sequence encoding the safety switch polypeptide comprising a suicide moiety;
(ii) the nucleotide sequence encoding FOXP3; and
(iii) the nucleotide sequence encoding a chimeric antigen receptor (CAR), may be operably linked to a SFFV promoter. The SFFV promoter may comprise a nucleotide sequence as set out in SEQ ID NO. 87. “Operably linked to the same promoter” means that transcription of the polynucleotide sequences may be initiated from the same promoter (e.g. transcription of the first, second and third polynucleotide sequences is initiated from the same promoter) and that the nucleotide sequences are positioned and oriented for transcription to be initiated from the promoter. Polynucleotides operably linked to a promoter are under transcriptional regulation of the promoter.

A vector is a tool that allows or facilitates the transfer of an entity from one environment to another. As used herein, and by way of example, some vectors used in recombinant nucleic acid techniques allow entities, such as a segment of nucleic acid (e.g. a heterologous DNA segment, such as a heterologous cDNA segment), to be transferred into a target cell. Vectors may be non-viral or viral. Examples of vectors used in recombinant nucleic acid techniques include, but are not limited to, plasmids, mRNA molecules (e.g. in vitro transcribed mRNAs), chromosomes, artificial chromosomes and viruses. The vector may also be, for example, a naked nucleic acid (e.g. DNA). In its simplest form, the vector may itself be a nucleotide of interest.

The vectors used herein may be, for example, plasmid, mRNA or virus vectors and may include a promoter (as described above) for the expression of a nucleic acid molecule/polynucleotide and optionally a regulator of the promoter.

In an embodiment the vector is a viral vector, for example a retroviral, e.g. a lentiviral vector or a gamma retroviral vector.

The vectors may further comprise additional promoters, for example, in one embodiment, the promoter may be a LTR, for example, a retroviral LTR or a lentiviral LTR. Long terminal repeats (LTRs) are identical sequences of DNA that repeat hundreds or thousands of times found at either end of retrotransposons or proviral DNA formed by reverse transcription of retroviral RNA. They are used by viruses to insert their genetic material into the host genomes. Signals of gene expression are found in LTRs: enhancer, promoter (can have both transcriptional enhancers or regulatory elements), transcription initiation (such as capping), transcription terminator and polyadenylation signal.

Suitably, the vector may include a 5′LTR and a 3′LTR.

The vector may comprise one or more additional regulatory sequences which may act pre- or post-transcriptionally. “Regulatory sequences” are any sequences which facilitate expression of the polypeptides, e.g. act to increase expression of a transcript or to enhance mRNA stability. Suitable regulatory sequences include for example enhancer elements, post-transcriptional regulatory elements and polyadenylation sites. Suitably, the additional regulatory sequences may be present in the LTR(s).

Suitably, the vector may comprise a Woodchuck Hepatitis Virus Posttranscriptional Regulatory Element (WPRE), e.g. operably linked to the promoter.

Vectors comprising the present nucleic acid molecules/polynucleotides may be introduced into cells using a variety of techniques known in the art, such as transformation and transduction. Several techniques are known in the art, for example infection with recombinant viral vectors, such as retroviral, lentiviral, adenoviral, adeno-associated viral, baculoviral and herpes simplex viral vectors; direct injection of nucleic acids and biolistic transformation.

Non-viral delivery systems include but are not limited to DNA transfection methods. Here, transfection includes a process using a non-viral vector to deliver a gene to a target cell. Non-viral delivery systems can include liposomal or amphipathic cell penetrating peptides, preferably complexed with a nucleic acid molecule or construct.

Typical transfection methods include electroporation, DNA biolistics, lipid-mediated transfection, compacted DNA-mediated transfection, liposomes, immunoliposomes, lipofectin, cationic agent-mediated transfection, cationic facial amphiphiles (CFAs) (Nat. Biotechnol. (1996) 14:556) and combinations thereof.

Although the present nucleic acid molecules are designed to be used as single constructs, and this would be contained in a single vector, it is not precluded that they are introduced into a cell in conjunction with other vectors, for example encoding other polypeptides it may be desired also to introduce into the cell.

Engineered cells may be generated by introducing a nucleic acid molecule, construct, or vector as defined herein, by one of many means including transduction with a viral vector, and transfection with DNA or RNA.

The present cell may be made by: introducing to a cell (e.g. by transduction or transfection) the nucleic acid molecule/polynucleotide, construct or vector as defined herein.

Suitable cells are discussed further below, but the cell may be from a sample isolated from a subject. The subject may be a donor subject, or a subject for therapy (i.e. the cell may be an autologous cell, or a donor cell, for introduction to another recipient, e.g. an allogeneic cell).

The cell may be generated by a method comprising the following steps:
(i) isolation of a cell-containing sample from a subject or provision of a cell-containing sample; and
(ii) introduction into (e.g. by transduction or transfection) the cell-containing sample of a nucleic acid molecule, construct, or vector as defined herein, to provide a population of engineered cells.

A target cell-enriched sample may be isolated from, enriched, and/or generated from the cell-containing sample prior to and/or after step (ii) of the method. For example, isolation, enrichment and/or generation of Tregs (or other target cells) may be performed prior to and/or after step (ii) to isolate, enrich or generate a Treg-enriched sample. Isolation and/or enrichment from a cell-containing sample may be performed after step (ii) to enrich for cells and/or Tregs (or other target cells) comprising the CAR, the nucleic acid molecule/polynucleotide, the construct and/or the vector as described herein.

A Treg-enriched sample may be isolated or enriched by any method known to those of skill in the art, for example by FACS and/or magnetic bead sorting. A Treg-enriched sample may be generated from the cell-containing sample by any method known to those of skill in the art, for example, from Tcon cells by introducing DNA or RNA coding for FOXP3 and/or from ex-vivo differentiation of inducible progenitor cells or embryonic progenitor cells. Methods for isolating and/or enriching other target cells are known in the art.

Suitably, an engineered target cell may be generated by a method comprising the following steps:
(i) isolation of a target-cell enriched sample from a subject or provision of a target cell-enriched sample; and
(ii) introduction into (e.g. by transduction or transfection) the target cell-enriched sample of a nucleic acid, construct or vector as defined herein, to provide a population of engineered target cells.

The target cell may be a Treg cell, or precursor or a progenitor therefor.

An “engineered cell” means a cell which has been modified to comprise or express a polynucleotide which is not naturally encoded by the cell. Methods for engineering cells are known in the art and include, but are not limited to, genetic modification of cells e.g. by transduction such as retroviral or lentiviral transduction, transfection (such as transient transfection-DNA or RNA based) including lipofection, polyethylene glycol, calcium phosphate and electroporation, as discussed above. Any suitable method may be used to introduce a nucleic acid sequence into a cell. Non-viral technologies such as amphipathic cell penetrating peptides may be used to introduce nucleic acid.

Accordingly, the nucleic acid molecule as described herein is not naturally expressed by a corresponding, unmodified cell. Indeed, the nucleic acid molecule encoding the CAR is an artificial construct, and in an embodiment the safety switch polypeptide is an artificial construct, such they could not occur or be expressed naturally. Suitably, an engineered cell is a cell which has been modified e.g. by transduction or by transfection. Suitably, an engineered cell is a cell which has been modified or whose genome has been modified e.g. by transduction or by transfection. Suitably, an engineered cell is a cell which has been modified or whose genome has been modified by retroviral transduction. Suitably, an engineered cell is a cell which has been modified or whose genome has been modified by lentiviral transduction.

As used herein, the term “introduced” refers to methods for inserting foreign nucleic acid, e.g. DNA or RNA, into a cell. As used herein the term introduced includes both transduction and transfection methods. Transfection is the process of introducing nucleic acids into a cell by non-viral methods. Transduction is the process of introducing foreign DNA or RNA into a cell via a viral vector. Engineered cells may be generated by introducing a nucleic acid as described herein by one of many means including transduction with a viral vector, transfection with DNA or RNA. Cells may be activated and/or expanded prior to, or after, the introduction of a nucleic acid as described herein, for example by treatment with an anti-CD3 monoclonal antibody or both anti-CD3 and anti-CD28 monoclonal antibodies. The cells may also be expanded in the presence of anti-CD3 and anti-CD28 monoclonal antibodies in combination with IL-2. Suitably, IL-2 may be substituted with IL-15. Other components which may be used in a cell (e.g. Treg) expansion protocol include, but are not limited to rapamycin, all-trans retinoic acid (ATRA) and TGFβ. As used herein “activated” means that a cell has been stimulated, causing the cell to proliferate. As used herein “expanded” means that a cell or population of cells has been induced to proliferate. The expansion of a population of cells may be measured for example by counting the number of cells present in a population. The phenotype of the cells may be determined by methods known in the art such as flow cytometry.

The cell may be an immune cell, or a precursor therefor. A precursor cell may be a progenitor cell. Representative immune cells thus include T-cells, in particular, cytotoxic T-cells (CTLs; CD8+ T-cells), helper T-cells (HTLs; CD4+ T-cells) and regulatory T cells (Tregs). Other populations of T-cells are also useful herein, for example naive T-cells and memory T-cells. Other immune cells include NK cells, NKT cells, dendritic cells, MDSC, neutrophils, and macrophages. Precursors of immune cells include pluripotent stem cells, e.g. induced PSC (iPSC), or more committed progenitors including multipotent stem cells, or cells which are committed to a lineage. Precursor cells can be induced to differentiate into immune cells in vivo or in vitro. In one aspect, a precursor cell may be a somatic cell which is capable of being transdifferentiated to an immune cell of interest.

Most notably, the immune cell may be an NK cell, a dendritic cell, a MDSC, or a T cell, such as a cytotoxic T lymphocyte (CTL) or a Treg cell.

In a preferred embodiment the immune cell is a Treg cell. “Regulatory T cells (Treg) or T regulatory cells” are immune cells with immunosuppressive function that control cytopathic immune responses and are essential for the maintenance of immunological tolerance. As used herein, the term Treg refers to a T cell with immunosuppressive function.

A T cell as used herein is a lymphocyte including any type of T cell, such as an alpha beta T cell (e.g. CD8 or CD4+), a gamma delta T cell, a memory T cell, a Treg cell

Suitably, immunosuppressive function may refer to the ability of the Treg to reduce or inhibit one or more of a number of physiological and cellular effects facilitated by the immune system in response to a stimulus such as a pathogen, an alloantigen, or an autoantigen. Examples of such effects include increased proliferation of conventional T cell (Tconv) and secretion of proinflammatory cytokines. Any such effects may be used as indicators of the strength of an immune response. A relatively weaker immune response by Tconv in the presence of Tregs would indicate an ability of the Treg to suppress immune responses. For example, a relative decrease in cytokine secretion would be indicative of a weaker immune response, and thus indicative of the ability of Tregs to suppress immune responses. Tregs can also suppress immune responses by modulating the expression of co-stimulatory molecules on antigen presenting cells (APCs), such as B cells, dendritic cells and macrophages. Expression levels of CD80 and CD86 can be used to assess suppression potency of activated Tregs in vitro after co-culture.

Assays are known in the art for measuring indicators of immune response strength, and thereby the suppressive ability of Tregs. In particular, antigen-specific Tconv cells may be co-cultured with Tregs, and a peptide of the corresponding antigen added to the co-culture to stimulate a response from the Tconv cells. The degree of proliferation of the Tconv cells and/or the quantity of the cytokine IL-2 they secrete in response to addition of the peptide may be used as indicators of the suppressive abilities of the co-cultured Tregs.

Antigen-specific Tconv cells co-cultured with Tregs as disclosed herein may proliferate 5%, 10%, 20%, 30%, 40%, 50%, 60%, 70%, 90%, 95% or 99% less than the same Tconv cells cultured in the absence of the Tregs. For example, antigen-specific Tconv cells co-cultured with the present Tregs may proliferate 5%, 10%, 20%, 30%, 40%, 50%, 60%, 70%, 90%, 95% or 99% less than the same Tconv cells cultured in the presence of non-engineered Tregs. The cells comprising the nucleic acid, expression construct or vector as defined herein, e.g. Tregs may have an increased suppressive activity as compared to non-engineered Tregs or to Tregs comprising a nucleic acid construct having a different arrangement of the three polynucleotide sequences encoding the three polypeptides (i.e. the safety switch polypeptide, FOXP3 and CAR), e.g. as compared to a construct comprising polynucleotides 5′ to 3′ encoding FOXP3, safety switch polypeptide and CAR. (e.g. an increased suppressive activity of at least 5, 10, 20, 30, 40, 50, 60, 70, 80 or 90%). Antigen-specific Tconv cells co-cultured with the Tregs herein may express at least 10%, at least 20%, at least 30%, at least 40%, at least 50%, or at least 60% less effector cytokine than corresponding Tconv cells cultured in the absence of the Tregs (e.g. in the presence of non-engineered Tregs, or Tregs comprising a nucleic acid construct having a different arrangement of the three polynucleotide sequences encoding the three polypeptides (i.e. the safety switch polypeptide, FOXP3 and CAR), e.g. as compared to a construct comprising polynucleotides 5′ to 3′ encoding FOXP3, safety switch polypeptide and CAR). The effector cytokine may be selected from IL-2, IL-17, TNFα, GM-CSF, IFN-γ, IL-4, IL-5, IL-9, IL-10 and IL-13. Suitably the effector cytokine may be selected from IL-2, IL-17, TNFα, GM-CSF and IFN-γ.

Several different subpopulations of Tregs have been identified which may express different or different levels of particular markers. Tregs generally are T cells which express the markers CD4, CD25 and FOXP3 (CD4+CD25+FOXP3+).

Tregs may also express CTLA-4 (cytotoxic T-lymphocyte associated molecule-4) or GITR (glucocorticoid-induced TNF receptor).

Treg cells are present in the peripheral blood, lymph nodes, and tissues and

Tregs for use herein include thymus-derived, natural Treg (nTreg) cells, peripherally generated Tregs, and induced Treg (iTreg) cells.

A Treg may be identified using the cell surface markers CD4 and CD25 in the absence of or in combination with low-level expression of the surface protein CD127 (CD4⁺CD25⁺CD127⁻ or CD4⁺CD25⁺CD127^{low}). The use of such markers to identify Tregs is known in the art and described in Liu et al. (JEM; 2006; 203; 7 (10); 1701-1711), for example.

A Treg may be a CD4⁺CD25⁺FOXP3⁺ T cell, a CD4⁺CD25⁺CD127⁻ T cell, or a CD4⁺CD25⁺FOXP3⁺CD127^{-/low} T cell.

Suitably, the Treg may be a natural Treg (nTreg). As used herein, the term “natural T reg” means a thymus-derived Treg. Natural T regs are CD4⁺CD25⁺FOXP3⁺ Helios⁺ Neuropilin 1⁺. Compared with iTregs, nTregs have higher expression of PD-1 (programmed cell death-1, pdcd1), neuropilin 1 (Nrp1), Helios (Ikzf2), and CD73. nTregs may be distinguished from iTregs on the basis of the expression of Helios protein or Neuropilin 1 (Nrp1) individually.

The Treg may have a demethylated Treg-specific demethylated region (TSDR). The TSDR is an important methylation-sensitive element regulating Foxp3 expression (Polansky, J. K., et al., 2008. European journal of immunology, 38 (6), pp. 1654-1663).

Further suitable Tregs include, but are not limited to, Tr1 cells (which do not express Foxp3, and have high IL-10 production); CD8⁺FOXP3⁺ T cells; and γδ FOXP3⁺ T cells.

Different subpopulations of Tregs are known to exist, including naïve Tregs (CD45RA⁺FoxP3^{low}), effector/memory Tregs (CD45RA⁻FoxP3^{high}) and cytokine-producing Tregs (CD45RA⁻FoxP3^{low}). “Memory Tregs” are Tregs which express CD45RO and which are considered to be CD45RO⁺. These cells have increased levels of CD45RO as compared to naïve Tregs (e.g. at least 10, 20, 30, 40, 50, 60, 70, 80 or 90% more CD45RO) and which preferably do not express or have low levels of CD45RA (mRNA and/or protein) as compared to naïve Tregs (e.g. at least 80, 90 or 95% less CD45RA as compared to naïve Tregs). “Cytokine-producing Tregs” are Tregs which do not express or have very low levels of CD45RA (mRNA and/or protein) as compared to naïve Tregs (e.g. at least 80, 90 or 95% less CD45RA as compared to naïve Tregs), and which have low levels of FOXP3 as compared to Memory Tregs, e.g. less than 50, 60, 70, 80 or 90% of the FOXP3 as compared to Memory Tregs. Cytokine-producing Tregs may produce interferon gamma and may be less suppressive in vitro as compared to naïve Tregs (e.g. less than 50, 60, 70, 80 or 90% suppressive than naïve Tregs. Reference to expression levels herein may refer to mRNA or protein expression. Particularly, for cell surface markers such as CD45RA, CD25, CD4, CD45RO etc, expression may refer to cell surface expression, i.e. the amount or relative amount of a marker protein that is expressed on the cell surface. Expression levels may be determined by any known method of the art. For example, mRNA expression levels may be determined by Northern blotting/array analysis, and protein expression may be determined by Western blotting, or preferably by FACS using antibody staining for cell surface expression.

Particularly, the Treg may be a naïve Treg. “A naïve regulatory T cell, a naïve T regulatory cell, or a naïve Treg” as used interchangeably herein refers to a Treg cell which expresses CD45RA (particularly which expresses CD45RA on the cell surface). Naïve Tregs are thus described as CD45RA+. Naïve Tregs generally represent Tregs which have not been activated through their endogenous TCRs by peptide/MHC, whereas effector/memory Tregs relate to Tregs which have been activated by stimulation through their endogenous TCRs. Typically, a naïve Treg may express at least 10, 20, 30, 40, 50, 60, 70, 80 or 90% more CD45RA than a Treg cell which is not naïve (e.g. a memory Treg cell). Alternatively viewed, a naïve Treg cell may express at least 2, 3, 4, 5, 10, 50 or 100-fold the amount of CD45RA as compared to a non-naïve Treg cell (e.g. a memory Treg cell). The level of expression of CD45RA can be readily determined by methods of the art, e.g. by flow cytometry using commercially available antibodies. Typically, non-naïve Treg cells do not express CD45RA or low levels of CD45RA.

Particularly, naïve Tregs may not express CD45RO, and may be considered to be CD45RO. Thus, naïve Tregs may express at least 10, 20, 30, 40, 50, 60, 70, 80 or 90% less CD45RO as compared to a memory Treg, or alternatively viewed at least 2, 3, 4, 5, 10, 50 or 100 fold less CD45RO than a memory Treg cell.

Although naïve Tregs express CD25 as discussed above, CD25 expression levels may be lower than expression levels in memory Tregs, depending on the origin of the naïve Tregs. For example, for naïve Tregs isolated from peripheral blood, expression levels of CD25 may be at least 10, 20, 30, 40, 50, 60, 70, 80 or 90% lower than memory Tregs. Such naïve Tregs may be considered to express intermediate to low levels of CD25. However, a skilled person will appreciate that naïve Tregs isolated from cord blood may not show this difference.

Typically, a naïve Treg as defined herein may be CD4⁺, CD25⁺, FOXP3⁺, CD127^{low}, CD45RA⁺.

Low expression of CD127 as used herein refers to a lower level of expression of CD127 as compared to a CD4⁺ non-regulatory or Tcon cell from the same subject or donor. Particularly, naïve Tregs may express less than 90, 80, 70, 60, 50, 40, 30, 20 or 10% CD127 as compared to a CD4⁺ non-regulatory or Tcon cell from the same subject or donor. Levels of CD127 can be assessed by methods standard in the art, including by flow cytometry of cells stained with an anti-CD127 antibody.

Typically, naïve Tregs do not express, or express low levels of CCR4, HLA-DR, CXCR3 and/or CCR6. Particularly, naïve Tregs may express lower levels of CCR4, HLA-DR, CXCR3 and CCR6 than memory Tregs, e.g. at least 10, 20, 30, 40, 50, 60, 70, 80 or 90% lower level of expression.

Naïve Tregs may further express additional markers, including CCR7⁺ and CD31⁺

Isolated naïve Tregs may be identified by methods known in the art, including by determining the presence or absence of a panel of any one or more of the markers discussed above, on the cell surface of the isolated cells. For example, CD45RA, CD4, CD25 and CD127 low can be used to determine whether a cell is a naïve Treg. Methods of determining whether isolated cells are naïve Tregs or have a desired phenotype can be carried out as discussed below in relation to additional steps which may be carried out, and methods for determining the presence and/or levels of expression of cell markers are well-known in the art and include, for example, flow cytometry, using commercially available antibodies.

Suitably, the cell, such as a Treg, is isolated from peripheral blood mononuclear cells (PBMCs) obtained from a subject. Suitably the subject from whom the PBMCs are obtained is a mammal, preferably a human. Suitably the cell is matched (e.g. HLA matched) or is autologous to the subject to whom the engineered cell is to be administered. Suitably, the subject to be treated is a mammal, preferably a human. The cell may be generated ex vivo either from a patient's own peripheral blood (1st party), or in the setting of a haematopoietic stem cell transplant from donor peripheral blood (2nd party), or peripheral blood from an unconnected donor (3rd party). Suitably the cell is autologous to the subject to whom the engineered cell is to be administered.

Suitably, the Treg is part of a population of cells. Suitably, the population of Tregs comprises at least 70% Tregs, such as at least 75, 85, 90, 95, 97, 98 or 99% Tregs. Such a population may be referred to as an “enriched Treg population”.

In some aspects, the Treg may be derived from ex-vivo differentiation of inducible progenitor cells (e.g. iPSCs) or embryonic progenitor cells to the Treg. A nucleic acid molecule or vector as described herein may be introduced into the inducible progenitor cells or embryonic progenitor cells prior to, or after, differentiation to a Treg. Suitable methods for differentiation are known in the art and include that disclosed in Haque et al, J Vis Exp., 2016, 117, 54720 (incorporated herein by reference).

As used herein, the term “conventional T cell” or Tcon or Tconv (used interchangeably herein) means a T lymphocyte cell which expresses an αβ T cell receptor (TCR) as well as a co-receptor which may be cluster of differentiation 4 (CD4) or cluster of differentiation 8 (CD8) and which does not have an immunosuppressive function. Conventional T cells are present in the peripheral blood, lymph nodes, and tissues. Suitably, the engineered Treg may generated from a Tcon by introducing the nucleic acid which includes a sequence coding for FOXP3 Alternatively, the engineered Treg may be generated from a Tcon by in vitro culture of CD4+ CD25−FOXP3− cells in the presence of IL-2 and TGF-β.

A Treg herein may have increased persistence as compared to a Treg cell without exogenous FOXP3. “Persistence” as used herein defines the length of time that Tregs can survive in a particular environment, e.g. in vivo (e.g. in a human patient or animal model). A Treg as disclosed herein may have at least 10, 20, 30, 40, 50, 60, 70, 80 or 90% increased persistence as compared to a Treg which does not comprise the nucleic acid molecule herein.

In another embodiment the target cell into which the nucleic acid molecule, construct or vector is introduced is not a cell intended for therapy. In an embodiment the cell is a production host cell. The cell may be for production of the nucleic acid, e.g. cloning, or vector, or polypeptides.

The invention also provides a cell population comprising a cell as defined or described herein. It will be appreciated that a cell population may comprise both cells of the invention comprising a nucleic acid molecule, expression construct or vector as defined herein, and cells which do not comprise a nucleic acid molecule, expression construct or vector of the invention, e.g. untransduced or untransfected cells. Although in a preferred embodiment, all the cells in a population may comprise a nucleic acid, expression construct or vector of the invention, cell populations having at least 10, 20, 30, 40, 50, 60, 70, 80, 90, 95 or 99% of cells comprising a nucleic acid, expression construct or vector of the invention are provided.

There is also provided a pharmaceutical composition comprising a cell or cell population as defined or described herein, a vector as defined herein. The vector may be used for gene therapy. Thus, rather than administering a cell, a vector may be administered instead, to modify endogenous cells in the subject to express the introduced nucleic acid molecule. Vectors suitable for use in gene therapy are known in the art, and include viral vectors.

A pharmaceutical composition is a composition that comprises or consists of a therapeutically effective amount of a pharmaceutically active agent i.e. the cell (e.g. Treg), cell population or vector. It preferably includes a pharmaceutically acceptable carrier, diluent or excipient (including combinations thereof). Acceptable carriers or diluents for therapeutic use are well known in the pharmaceutical art, and are described, for example, in Remington's Pharmaceutical Sciences, Mack Publishing Co. (A. R. Gennaro edit. 1985). The choice of pharmaceutical carrier, excipient or diluent can be selected with regard to the intended route of administration and standard pharmaceutical practice. The pharmaceutical compositions may comprise as—or in addition to—the carrier, excipient or diluent any suitable binder(s), lubricant(s), suspending agent(s), coating agent(s) or solubilising agent(s).

By “pharmaceutically acceptable” is included that the formulation is sterile and pyrogen free. The carrier, diluent, and/or excipient must be “acceptable” in the sense of being compatible with the cell or vector and not deleterious to the recipients thereof. Typically, the carriers, diluents, and excipients will be saline or infusion media which will be sterile and pyrogen free, however, other acceptable carriers, diluents, and excipients may be used.

Examples of pharmaceutically acceptable carriers include, for example, water, salt solutions, alcohol, silicone, waxes, petroleum jelly, vegetable oils, polyethylene glycols, propylene glycol, liposomes, sugars, gelatin, lactose, amylose, magnesium stearate, talc, surfactants, silicic acid, viscous paraffin, perfume oil, fatty acid monoglycerides and diglycerides, petroethral fatty acid esters, hydroxymethyl-cellulose, polyvinylpyrrolidone, and the like.

The cells, cell population or pharmaceutical compositions may be administered in a manner appropriate for treating and/or preventing the desired disease or condition. The quantity and frequency of administration will be determined by such factors as the condition of the subject, and the type and severity of the subject's disease or condition, although appropriate dosages may be determined by clinical trials. The pharmaceutical composition may be formulated accordingly.

The cell, cell population or pharmaceutical composition as described herein can be administered parenterally, for example, intravenously, or they may be administered by infusion techniques. The cell, cell population or pharmaceutical composition may be administered in the form of a sterile aqueous solution which may contain other substances, for example, enough salts or glucose to make the solution isotonic with blood. The aqueous solution may be suitably buffered (preferably to a pH of from 3 to 9). The pharmaceutical composition may be formulated accordingly. The preparation of suitable parenteral formulations under sterile conditions is readily accomplished by standard pharmaceutical techniques well-known to those skilled in the art.

The pharmaceutical compositions may comprise cells in infusion media, for example sterile isotonic solution. The pharmaceutical composition may be enclosed in ampoules, disposable syringes or multiple dose vials made of glass or plastic.

The cell, cell population or pharmaceutical composition may be administered in a single or in multiple doses. Particularly, the cell, cell population or pharmaceutical composition may be administered in a single, one off dose. The pharmaceutical composition may be formulated accordingly.

The pharmaceutical composition may further comprise one or more active agents. The pharmaceutical composition may further comprise one or more other therapeutic agents, such as lympho-depletive agents (e.g. thymoglobulin, campath-1H, anti-CD2 antibodies, anti-CD3 antibodies, anti-CD20 antibodies, cyclophosphamide, fludarabine), inhibitors of mTOR (e.g. sirolimus, everolimus), drugs inhibiting costimulatory pathways (e.g. anti-CD40/CD40L, CTAL4Ig), and/or drugs inhibiting specific cytokines (IL-6, IL-17, TNFalpha, IL18).

Depending upon the disease/condition and subject to be treated, as well as the route of administration, the cell, cell population or pharmaceutical composition may be administered at varying doses (e.g. measured in cells/kg or cells/subject). The physician in any event will determine the actual dosage which will be most suitable for any individual subject and it will vary with the age, weight and response of the particular subject. Typically, however, for the cells herein, doses of 5×10⁷ to 3×10⁹ cells, or 10⁸ to 2×10⁹ cells per subject may be administered.

The cell may be appropriately modified for use in a pharmaceutical composition. For example, cells may be cryopreserved and thawed at an appropriate time, before being infused into a subject.

The invention further includes the use of kits comprising the cell, cell population and/or pharmaceutical composition herein. Preferably said kits are for use in the methods and uses as described herein, e.g., the therapeutic methods as described herein. Preferably said kits comprise instructions for use of the kit components.

The cells, cell populations, compositions and vectors herein may be for use in treating or preventing a disease a condition, notably a disease or condition which may be treated by or with the CAR. The cells and compositions containing them are for adoptive cell therapy (ACT). Various conditions may be treated by administration of cells, including particularly Treg cells, expressing a CAR according to the present disclosure. As noted above, this may be conditions responsive to immunosuppression, and particularly the immunosuppressive effects of Tregs cells. The cells, cell populations, compositions and vectors described herein may thus be used for inducing, or achieving, immunosuppression in a subject. The Treg cells administered, or modified in vivo, may be targeted by expression of the CAR. Conditions suitable for such treatment include infectious, neurodegenerative or inflammatory disease, or more broadly a condition associated with any undesired or unwanted or deleterious immune response.

Conditions to be treated or prevented include inflammation, or alternatively put, a condition associated with or involving inflammation. Inflammation may be chronic or acute. Furthermore, the inflammation may be low-level or systemic inflammation. For example the inflammation may be inflammation which occurs in the context of a metabolic disorder, for example metabolic syndrome, or in the context of insulin resistance, or type II diabetes or obesity and such like.

In particular, the cells, cell populations, vectors and pharmaceutical compositions provide a means for inducing tolerance to a transplant; treating and/or preventing cellular and/or humoral transplant rejection; treating and/or preventing graft-versus-host disease (GvHD), an autoimmune or allergic disease; or to promote tissue repair and/or tissue regeneration; or to ameliorate inflammation. The cells, cell populations, vectors and pharmaceutical compositions may be used in a method which comprises the step of administering a cell, cell populations, vector or a pharmaceutical composition as described herein to a subject.

As used herein, “inducing tolerance to a transplant” refers to inducing tolerance to a transplanted organ in a recipient. In other words, inducing tolerance to a transplant means to reduce the level of a recipient's immune response to a donor transplant organ. Inducing tolerance to a transplanted organ may reduce the amount of immunosuppressive drugs that a transplant recipient requires, or may enable the discontinuation of immunosuppressive drugs.

For example, the engineered cells, e.g. Tregs, may be administered to a subject with a disease in order to lessen, reduce, or improve at least one symptom of disease such as jaundice, dark urine, itching, abdominal swelling or tenderness, fatigue, nausea or vomiting, and/or loss of appetite. The at least one symptom may be lessened, reduced, or improved by at least 10%, at least 20%, at least 30%, at least 40%, or at least 50%, or the at least one symptom may be completely alleviated.

The engineered cells, e.g. Tregs may be administered to a subject with a disease in order to slow down, reduce, or block the progression of the disease. The progression of the disease may be slowed down, reduced, or blocked by at least 10%, at least 20%, at least 30%, at least 40%, or at least 50% compared to a subject in which the engineered cells are not administered, or progression of the disease may be completely stopped.

In one embodiment, the subject is a transplant recipient undergoing immunosuppression therapy.

Suitably, the subject is a mammal. Suitably, the subject is a human.

The transplant may be selected from a liver, kidney, heart, lung, pancreas, intestine, stomach, bone marrow, vascularized composite tissue graft, and skin transplant.

Suitably, the CAR may comprise an antigen binding domain which is capable of specifically binding to a HLA antigen that is present in the graft (transplant) donor but not in the graft (transplant) recipient.

Suitably, the transplant is a liver transplant. In embodiments where the transplant is a liver transplant, the antigen may be a HLA antigen present in the transplanted liver but not in the patient, a liver-specific antigen such as NTCP, or an antigen whose expression is up-regulated during rejection such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11.

As discussed above, in one representative and preferred embodiment the antigen is HLA-A2.

A method for treating a disease or condition relates to the therapeutic use of the cells herein. In this respect, the cells may be administered to a subject having an existing disease or condition in order to lessen, reduce or improve at least one symptom associated with the disease or condition and/or to slow down, reduce or block the progression of the disease.

Suitably, treating and/or preventing cellular and/or humoral transplant rejection may refer to administering an effective amount of the cells (e.g. Tregs) such that the amount of immunosuppressive drugs that a transplant recipient requires is reduced, or may enable the discontinuation of immunosuppressive drugs.

Preventing a disease or condition relates to the prophylactic use of the cells herein. In this respect, the cells may be administered to a subject who has not yet contracted or developed the disease or condition and/or who is not showing any symptoms of the disease or condition to prevent the disease or condition or to reduce or prevent development of at least one symptom associated with the disease or condition. The subject may have a predisposition for, or be thought to be at risk of developing, the disease or condition.

The autoimmune or allergic disease may be selected from inflammatory skin diseases including psoriasis and dermatitis (e.g. atopic dermatitis); responses associated with inflammatory bowel disease (such as Crohn's disease and ulcerative colitis); dermatitis; allergic conditions such as food allergy, eczema and asthma; rheumatoid arthritis; systemic lupus erythematosus (SLE) (including lupus nephritis, cutaneous lupus); diabetes mellitus (e.g. type 1 diabetes mellitus or insulin dependent diabetes mellitus); multiple sclerosis; neurodegenerative disease, for example, Amyotrophic Lateral Sclerosis (ALS); Chronic inflammatory demyelinating polyneuropathy (CIPD) and juvenile onset diabetes.

The medical use of or method herein may involve the steps of:
(i) isolating a cell-containing sample or providing a cell-containing sample;
(ii) introducing a nucleic acid molecule, construct or a vector as defined herein to the cell; and
(iii) administering the cells from (ii) to a subject.

The cell may be a Treg as defined herein. An enriched Treg population may be isolated and/or generated from the cell containing sample prior to, and/or after, step (ii) of the method. For example, isolation and/or generation may be performed prior to and/or after step (ii) to isolate and/or generate an enriched Treg sample. Enrichment may be performed after step (ii) to enrich for cells and/or Tregs comprising the CAR, the polynucleotide, and/or the vector as described herein.

Suitably, the cell may be autologous. Suitably, the cell may be allogenic. Suitably, the cell (e.g. the engineered Treg) may be administered is combination with one or more other therapeutic agents, such as lympho-depletive agents (e.g. as discussed above). The engineered cell, e.g. Treg, may be administered simultaneously with or sequentially with (i.e. prior to or after) the one or more other therapeutic agents.

Suitably the subject is a mammal. Suitably the subject is a human.

Cells, e.g. Tregs, may be activated and/or expanded prior to, or after, the introduction of a nucleic acid molecule as described herein, for example by treatment with an anti-CD3 monoclonal antibody or both anti-CD3 and anti-CD28 monoclonal antibodies. Expansion protocols are discussed above.

The cell, e.g. Tregs, may be washed after each step of the method, in particular after expansion.

The population of engineered cells, e.g., Treg cells may be further enriched by any method known to those of skill in the art, for example by FACS or magnetic bead sorting.

The steps of the method of production may be performed in a closed and sterile cell culture system.

The present invention also provides a method or a means for deleting cells which express a safety switch polypeptide as defined herein, e.g. at the cell surface. The cell may be cell which comprises a nucleic acid molecule or vector or recombinant construct as defined or described herein, i.e. a cell into which the nucleic acid molecule or vector or construct has been introduced e.g. a cell transduced by a vector herein.

Generally, the method comprises exposing the target cells to a condition or agent which allows the safety switch polypeptide to elicit its effect. Such conditions are permissive conditions as referred to herein. This may involve the contact of the cells with, including the administration to the subject of, an agent which acts together with the safety switch polypeptide to cause deletion of the cell. For example, the agent may be an activating molecule or substrate for the safety switch polypeptide. In the context of the Rituximab-epitope containing switches described above, the method comprises the step of exposing the cells to Rituximab, or an antibody having the binding specificity of Rituximab (i.e. an equivalent antibody).

Typically, Rituximab exerts its effects through complement-mediated cell killing, although other mechanisms may be involved, for example ADCC. Accordingly, in one embodiment the cells may be exposed to complement and Rituximab, or an equivalent antibody.

The method includes a method carried out in vitro to delete cells, e.g. in culture. However, the primary use is to delete cells in vivo, i.e. to delete cells which have previously been administered to a subject.

It will be appreciated that in vivo this may be achieved by administering the Rituximab or equivalent antibody to a subject to whom the cell has previously been administered, in other words a subject who has previously received ACT with a cell as defined herein which expresses the safety switch polypeptide or a vector for modification of endogenous cells to express the safety with polypeptide. Complement may be present endogenously in the subject.

Thus, Rituximab, or an antibody having the binding specificity thereof, may be provided for use in ACT in combination with a cell of the invention. The cell or nucleic acid or vector or construct for production of the cell and the Rituximab or equivalent antibody may be provided in a kit, or as a combination product.

When the safety switch polypeptide is expressed at the surface of a cell, binding of Rituximab or equivalent antibody to the R epitopes of the polypeptide causes lysis of the cell.

An antibody which has the binding specificity of Rituximab is an antibody which is capable of binding to the same natural epitope as does Rituximab. In particular, the antibody is capable of binding to the epitopes R1 and R2.

An antibody having the binding specificity of Rituximab may comprise an antigen binding domain of or from Rituximab. More particularly, it may comprise a VL and a VH domain from Rituximab, or the CDRs of Rituximab. Further, the antigen binding domain of Rituximab may be modified (e.g. by amino acid substitution, deletion or insertion) as long as the binding specificity of Rituximab is retained.

As noted above, biosimilars for Rituximab are available and may be used. A person of skill in the art is readily able to use routine methods to prepare an antibody having the binding specificity of Rituximab using the available amino acid sequences therefor.

In an embodiment the antibody having the binding specificity of Rituximab is in the conventional immunoglobulin format. That is it may comprise light and heavy chains and both constant and variable regions. The antibody may be bivalent, that is it may comprise two antigen binding sites. Other antibody formats may also be used, including for example a single chain format, or a monovalent format. The antibody may thus be of any class or type, or format.

More than one molecule of Rituximab or equivalent antibody may bind per safety switch polypeptide expressed at the cell surface. Each R epitope of the polypeptide may bind a separate molecule of Rituximab or equivalent antibody.

The decision to delete the transferred cells may arise from undesirable effects being detected in the subject which are attributable to the transferred cells. For example, unacceptable levels of toxicity may be detected.

CD20-expressing cells may be selectively ablated by treatment with the antibody Rituximab. As CD20 expression is absent from plasma cells, humoral immunity is retained following Rituximab treatment despite deletion of the B-cell compartment.

The invention may also provide a method for increasing the stability and/or suppressive function of a cell comprising the step of introducing a nucleic acid molecule, an expression construct or vector as provided herein into the cell. An increase in suppressive function can be measured as discussed above, for example by co-culturing activated antigen-specific Tconv cells with cells of the invention, and for example measuring the levels the cytokines produced by the Tconv cells. An increase in suppressive function may be an increase of at least 10, 20, 30, 40, 50, 60, 70, 80, or 90% as compared to a non-engineered Treg or a Treg comprising a nucleic acid construct having a different arrangement of the three polynucleotide sequences encoding the three polypeptides (i.e. the safety switch polypeptide, FOXP3 and CAR), e.g. a construct comprising polynucleotides 5′ to 3′ encoding FOXP3, safety switch polypeptide and CAR.

An increase in stability of a cell, e.g. a Treg as defined herein, refers to an increase in the persistence or survival of those cells or to an increase in the proportion of cells retaining a Treg phenotype over a time period (e.g. to cells retaining Treg markers such as FOXP3 and Helios) as compared to a non-engineered Treg or a Treg comprising a nucleic acid construct having a different arrangement of the three polynucleotide sequences encoding the three polypeptides (i.e. the safety switch polypeptide, FOXP3 and CAR), e.g. a construct comprising polynucleotides 5′ to 3′ encoding FOXP3, safety switch polypeptide and CAR. An increase in stability may be an increase in stability of at least 10, 20, 30, 40, 50, 60, 70, 80 or 90%, and may be measured by techniques known in the art, e.g. staining of Treg cell markers within a population of cells, and analysis by FACS.

The invention may also provide for a method of enhancing the expression of FOXP3 from a nucleic acid molecule encoding a chimeric antigen receptor (CAR), a safety switch and FOXP3 in a cell, comprising selecting a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a CAR,
and introducing said nucleic acid molecule into said cell.

The method may further include a step of producing the nucleic acid molecule (e.g. of positioning a nucleotide sequence encoding FOXP3 downstream of a nucleotide sequence encoding a safety switch polypeptide comprising a suicide moiety and upstream of a nucleotide sequence encoding a CAR). Thus, it is the 5′ to 3′ order of (i), (ii) and (iii) as set out above within the nucleic acid molecule which provides the enhanced expression of FOXP3 after introduction of the nucleic acid molecule into the cell. As discussed previously, particularly the CAR may target HLA A2. Additionally or alternatively, the nucleic acid molecule may comprise a SFFV promoter, wherein (i), (ii) and (ii) may be operably linked to said promoter.

In an additional aspect, the present invention provides the use of a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR) for expression of a suicide moiety, FOXP3 and a CAR within a cell, wherein expression of FOXP3 is enhanced.

The method or use relates to “enhancing” the expression of FOXP3 from a nucleic acid molecule encoding a safety switch, CAR and FOXP3. In this respect, “increasing” or “improving” may be used interchangeably with “enhancing” and as discussed previously above relates to an increased level of mRNA or protein of FOXP3 within a transduced cell, as compared to expression levels of FOXP3 mRNA or protein obtained in a cell transduced using a nucleic acid molecule wherein (i), (ii) and (iii) are placed in a different order to the nucleic acid molecule of the invention (i.e. are not in the 5′ to 3′ order of (i), (ii) and (iii)). A nucleic acid molecule of the invention can thus be used to enhance expression of FOXP3 in a cell, as compared to other comparative nucleic acid molecules comprising the same nucleotide sequences encoding a safety switch polypeptide, FOXP3 and a CAR but present in a different gene order.

In another aspect, the inventors have further shown that a cell transduced with a nucleic acid molecule (e.g. a nucleic acid molecule of the invention) expressing a safety switch polypeptide comprising a suicide moiety of at least one CD20 epitope recognised by Rituximab may have an increased sensitivity to Rituximab when the nucleotide sequence encoding the safety switch polypeptide is operably linked to a SFFV promoter. In this regard, the invention additionally provides a method of increasing the sensitivity of a cell expressing a safety switch polypeptide comprising a suicide moiety of at least one CD20 epitope recognised by Rituximab, to Rituximab, comprising introducing into said cell a nucleic acid molecule comprising a nucleotide sequence encoding said safety switch polypeptide wherein said nucleotide sequence is operably linked to a SFFV promoter.

Alternatively viewed, the invention provides for a method of increasing the sensitivity of a cell expressing a safety switch polypeptide comprising a suicide moiety of at least one CD20 epitope recognised by Rituximab, to Rituximab, comprising expressing said safety switch polypeptide from a nucleotide sequence operably linked to a SFFV promoter. Particular safety switch polypeptides comprising a CD20 epitope recognised by Rituximab are as described previously above.

The invention further provides a use of a nucleic acid molecule comprising a nucleotide sequence operably linked to a SFFV promoter, wherein said nucleotide sequence encodes a safety switch polypeptide comprising a suicide moiety of at least one CD20 epitope recognised by Rituximab, for increasing the sensitivity of a cell to Rituximab.

“Increasing the sensitivity of a cell to Rituximab” as used herein means to increase the ability of a cell to be deleted or depleted by Rituximab. Thus, increasing the sensitivity to Rituximab can in one aspect refer to the increased ability of a particular concentration of Rituximab to delete or deplete cells (e.g. to delete or deplete an increased number or percentage of cells or to delete or deplete a particular number or percentage of cells over a shorter time period). Alternatively viewed, increasing the sensitivity to Rituximab can refer to the ability of reduced concentrations of Rituximab to delete or deplete cells. The increased sensitivity of a cell to deletion or depletion by Rituximab, is typically in comparison with a cell transduced with a nucleic acid molecule comprising a nucleotide sequence encoding a safety switch polypeptide comprising a suicide moiety of at least one CD20 epitope recognised by Rituximab (particularly the same nucleotide sequence as the nucleic acid described above), wherein said nucleotide sequence is operably linked to a promoter which is not SFFV, e.g. to a promoter which is EFS or PGS. Thus, particularly, increased sensitivity may refer to the increased percentage deletion or depletion obtained in a cell population expressing the safety switch polypeptide under the control of an SFFV promoter after treatment with a particular concentration of Rituximab as compared to the percentage deletion or depletion obtained in a cell population expressing the safety switch polypeptide under the control of a promoter which is not SFFV (e.g. EFS or PGS) after treatment with the same concentration of Rituximab. Typically, the increased percentage deletion of depletion may be at least 10, 20, 30, 40, 50 or 60%. Alternatively, increased sensitivity to Rituximab can refer to the ability of reduced concentrations of Rituximab (e.g., 10, 20, 30, 40, 50, 60, 70, 80 or 90% less Rituximab) to delete or deplete cells in a cell population expressing the safety switch polypeptide under the control of an SFFV promoter as compared to the concentration of Rituximab required to achieve the same level (i.e. the same amount) of deletion or depletion in a cell population expressing the safety switch polypeptide under the control of a promoter which is not SFFV (e.g. EFS or PGS).

Deletion or depletion of cells can be determined and measured as described previously above.

This disclosure is not limited by the exemplary methods and materials disclosed herein, and any methods and materials similar or equivalent to those described herein can be used in the practice or testing of embodiments of this disclosure. Numeric ranges are inclusive of the numbers defining the range. Unless otherwise indicated, any nucleic acid sequences are written left to right in 5′ to 3′ orientation; amino acid sequences are written left to right in amino to carboxy orientation, respectively.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within this disclosure. The upper and lower limits of these smaller ranges may independently be included or excluded in the range, and each range where either, neither or both limits are included in the smaller ranges is also encompassed within this disclosure, subject to any specifically excluded limit in the stated range. Where the stated range includes one or both of the limits, ranges excluding either or both of those included limits are also included in this disclosure. It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes” or “containing”, “contains”, and are inclusive or open-ended and do not exclude additional, non-recited members, elements or method steps. The terms “comprising”, “comprises” and “comprised of” also include the term “consisting of”.

The publications discussed herein are provided solely for their disclosure prior to the filing date of the present application. Nothing herein is to be construed as an admission that such publications constitute prior art to the claims appended hereto. The invention will now be further described by way of Examples, which are meant to serve to assist one of ordinary skill in the art in carrying out the invention and are not intended in any way to limit the scope of the invention.

The invention will now be further described by way of Examples, which are meant to serve to assist one of ordinary skill in the art in carrying out the invention and are not intended in any way to limit the scope of the invention.

### Example 1

### Materials and Methods

### Cloning:

10 constructs (FIG. 1) were designed in house, and whole sequences were codon optimised for expression in human cells and manufactured. Constructs were cloned into the pMP71 backbone and D5a high efficiency bacteria were transformed with plasmid and grown with the selection agent ampicillin. DNA was extracted using a Miniprep Kit (Qiagen). Inserts were transferred into a lentiviral backbone by PCR cloning. Construct 1 represents a construct of the present disclosure as claimed herein. Constructs II-VI are comparative constructs and constructs VII-X are control constructs.

### Collection of PBMCs:

Leukocyte cones were supplied by NHS blood and transplant. PBMC were isolated using a density centrifugation protocol. Briefly, blood was diluted 1:1 with 1×PBS and layered over Ficoll-Paque (GE Healthcare). Samples were centrifuged and the leukocyte layer was removed and washed in PBS.

### Treg and Tconv Isolation Protocol:

Blood cones from HLA-A*02 negative donors were used to derive Treg and Teff populations. Blood cones were subjected to CD4 enrichment via negative selection using RosetteSep™ Human CD4+ T Cell Enrichment Cocktail. Subsequently, CD4+ cells were isolated using density centrifugation. CD4+CD25+ T cells were then isolated via positive selection using CD25 microbeads II (Miltenyi). The CD4+ CD25− fraction of cells were retained to serve as conventional T cell (Tconv) populations. The CD4+CD25+ fraction was stained with flow cytometry antibodies CD4 FITC (OKT4, Biolegend), CD25 PE-Cy7 (BC96, Biolegend), CD127 BV421 (A019D5, Biolegend), CD45RA BV510 (HI100, Biolegend) and the LIVE/DEAD™ Fixable Near-IR—Dead Cell Stain (Thermofisher) before FACS sorting. Where indicated, CD4+CD25+ CD127low (Bulk Tregs) or CD4+CD25+CD127low CD45RA+ (CD45RA+ Tregs) were sorted and used.

### Phoenix Cells

The retroviral packaging line Phoenix-GP (stably expressing Gag Pol) were purchased from ATCC and cultured.

### T Conv Culture Media:

Human Tconv were grown in RPMI-1640 (Gibco) supplemented with 10% heat inactivated foetal bovine serum; penicillin; streptomycin; L-glutamine (Gibco).

### T Reg Culture Media and Expansion:

Human Regulatory T cells were cultured in Texmacs media (Miltenyi) supplemented with IL-2 and activated with Human T-Activator CD3/CD28 Dynabeads™ (Gibco). Cells were re-fed every 2 to 3 days with Treg culture media supplemented with IL-2. A second round of stimulation with Dynabeads™ was performed to promote further expansion of Treg cells.

### Transfection and Viral Particle Production

### Retrovirus:

Phoenix-GP cells were seeded and cultured for 24 hours. On day 1 media was changed before transfection with relevant plasmid DNA and envelope plasmid. Transfection mixture was added drop-wise over the entire surface of the adherent Phoenix cells and incubated for a further 24 hours. On day 2 media was changed and incubated for a further 24 hours. On day 3 supernatant containing retrovirus was removed from the Phoenix cells and centrifuged to remove any cellular debris.

### Lentivirus:

HEK293T cells were seeded and cultured in DMEM (Dulbecco's Modified Eagle's Medium)+10% Fetal Bovine Serum (FBS) for 24 hours. Transfection reagents were brought to room temperature and were mixed with DNA construct/plasmid of interest, packaging plasmid (pD8.91) and viral envelope (pVSV-G). PEI was added to the diluted DNA and mixed and added to HEK293 Ts. Supernatant was harvested 48 hours post-transfection, filtered and virus concentrated.

### Transduction of T Cells

Tconv were activated with anti-CD3 and anti-CD28 Dynabeads (Gibco) and resuspended in T cell culture media. Non-tissue culture-treated 24-well plates were prepared by coating with Retronectin (Takahara-bio-Otsu, Japan), cell suspension was added together with retroviral/lentiviral supernatant. Cells were incubated and media exchange was performed on alternate days. Cells were used for experiments 7 days post-transduction.

### Flow Cytometry Staining to Determine CAR and RQR8 Expression

T cells were removed from culture and washed in FACS buffer and stained for HLA-A*02 specific CAR using an HLA-A*02 specific Dextramer (WB2720-APC, Immudex) in FACS buffer. Subsequently, cells were stained with the LIVE/DEAD™ Fixable Near-IR-Dead Cell Stain (Thermofisher) in PBS first and then with anti-CD4 AF700 (RPA-T4, BD), anti-CD34 FITC (QBEND/10, Thermofisher) and anti-CD3 PE-Cy7 in FACS staining buffer. For intracellular staining of FOXP3, cells were fixed and permeabilized and stained with the anti-Foxp3 PE (150D/E4, Thermofisher) antibody. Cells were analysed on a BD LSRII flow cytometer.

### Flow Cytometry Phenotyping of Transduced Cells

T cells were removed from culture and stained for the CAR and live cells using the Dextramer and LIVE/DEAD™ Fixable Near-IR-Dead Cell Stain as described above. Surface staining of the cells was performed using anti-CD4 AF700, anti-CD25 PE-Cy7 (BC96, Biolegend), anti-CD39 PerCPCy5.5 (A1, Biolegend), anti-CD62L PE-CF594 (DREG-56, BD), anti-TIM3 BV786 (7D3, BD), anti-TIGIT BV605 (A15153G, Biolegend), anti-CD45RO BUV395 (UCHL1, BD), anti-CD279 BUV737 (EH12.1, BD) and anti-CD223 BV711 (11C3C65, Biolegend). Cells were permeabilised and stained with anti-Foxp3 PE (150D/E4, Thermofisher).

### IL-2 Starvation Assay

Transduced Treg cells expanded in culture until day 15 were removed from culture and enriched using magnetic activated cell sorting (Miltenyi). Briefly, cells were first stained with anti-CD34 (QBEND/10)—FITC and subsequently stained with Anti-FITC Microbeads (Miltenyi). The cells were then passed through the MS columns (Miltenyi) along the octoMACS magnet (Miltenyi) to separate the RQR8 expressing cells. These enriched T cells were cultured with Human T-Activator CD3/CD28 Dynabeads™ with varying concentrations of IL-2. The IL-2 concentrations tested were 300 IU/ml, 150 IU/ml, 75 IU/ml, 30 IU/ml, 10 IU/ml and 1 IU/ml. Following incubation, cells were removed and stained to determine CAR and RQR8 expression as described above. Dead cells were identified using the dead cell stain as described.

### CAR-Specific Treg Activation Assay

Transduced Tregs were expanded in culture for 14 days and rested for 24 hours before culturing with anti-CD3/28 beads, K562 cells expressing HLA-A1 (A1) or HLA-A2 (A2) for a further 18 hours. Cells were stained with fixable viability dye, CD4, RQR8 and CD69.

### CAR-Specific Activation and Proliferation Assay

Tregs were isolated, transduced with construct I and expanded for 14 days. To perform the assay, cells were rested for 24 hours before being stained with Cell Trace Violet (CTV) and cultured with anti-CD3/28 beads, K562 cells expressing HLA-A1 (A1) or HLA-A2 (A2) for 5 days. Cells were stained with fixable viability dye, CD4 and RQR8.

### RQR8 Functional Assessment

CD3+ lymphocytes from the spleens and lymph nodes of CD45.1 mice were isolated and transduced with a construct expressing RQR8. Male CD45.2 mice were irradiated and 24 hours later 10×10-6 transduced cells were transferred via intravenous injection. At day 7 post-transfer blood was collected from the mice via tail vein bleed. Subsequently half of the mice were treated with murine aCD20 antibody (150 ug/mouse) on day 8, 11 and 13. On day 15, all mice were sacrificed and liver, spleen and blood tissues harvested. Tissues were processed to single cell suspension and prepared for flow cytometry analysis by staining with CD45.1 and RQR8 (Qbend).

### Rituximab Mediated CAR-Treg Depletion

After 14 days of expansion Tregs transduced with SFFV or EFS were incubated with complement and decreasing concentrations of Rituximab antibody for 4 hours. Cells were stained with fixable live/dead dye, CD4 and RQR8.

### Data Analysis

Flow cytometric data was analysed using the Flow Cytometry Analysis software FlowJo (Flowjo, LLC). All statistical analyses were performed using Graphpad Prism v.5 (Graphpad, Software).

### Results

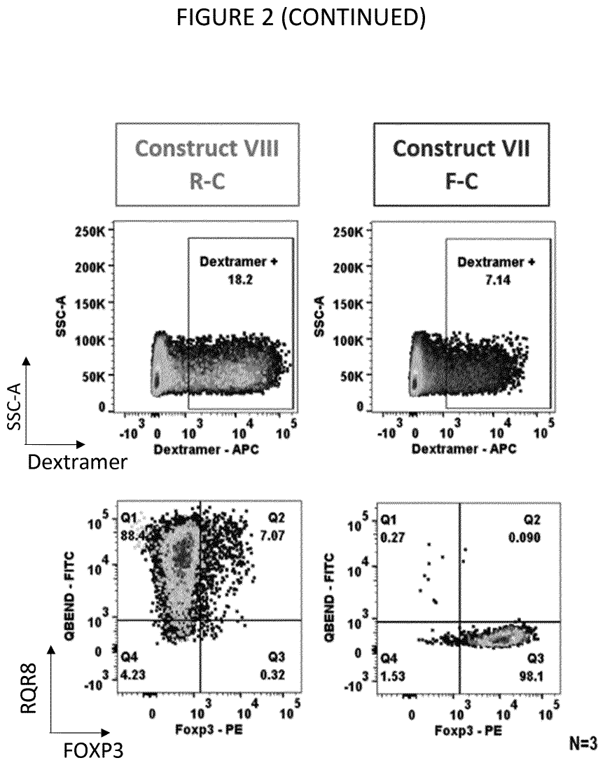

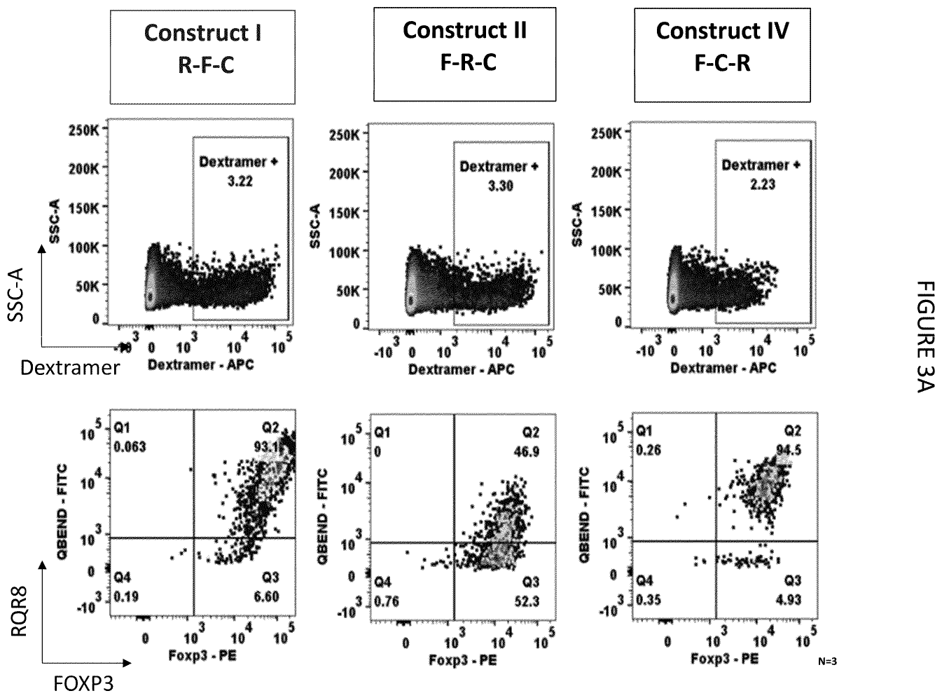

FIG. 2 shows the differential expression of the components encoded by the experimental and control constructs of FIG. 1 in T-effector cells (transduced with γ-retrovirus (γRV), as detected by expression of the encoded components of each construct. Detection (i.e. presence) of Dextramer confirms expression of the CAR, and expression of FOXP3 and the safety switch polypeptide (RQR8) is shown. Construct I shows expression of CAR (detection of Dextramer), safety switch polypeptide and FOXP3. Dextramer levels are particularly high for construct I as compared to constructs II and IV, and FOXP3 levels for construct I are significantly increased.

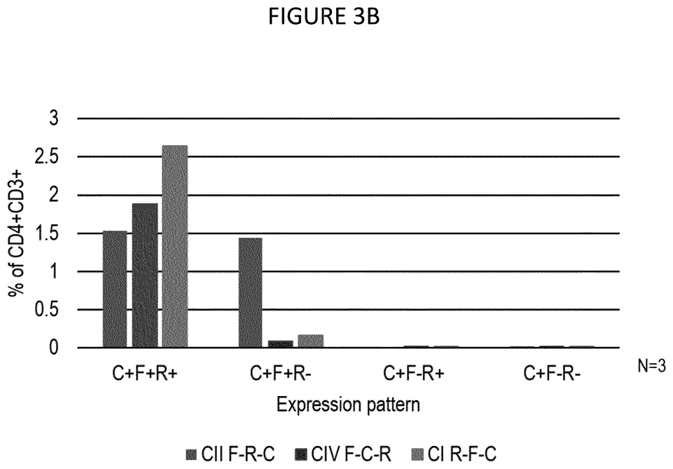

FIGS. 3 A and B shows that the expression patterns are maintained in Tregs low-transduced with γRV. As shown in FIG. 2 and confirmed in FIG. 3, expression of the safety switch and particularly of FOXP3 is increased from construct I compared to other constructs. In particular, construct I shows strong expression of C+F+R+, that is all 3 desired components, without associated expression of undesired variants such as C+F+R−, as shown in FIG. 3B. The expression of the desired expression pattern is increased from construct I. With other constructs which encode the components in a different order the desired expression pattern is reduced and increased undesired expression patterns may be seen.

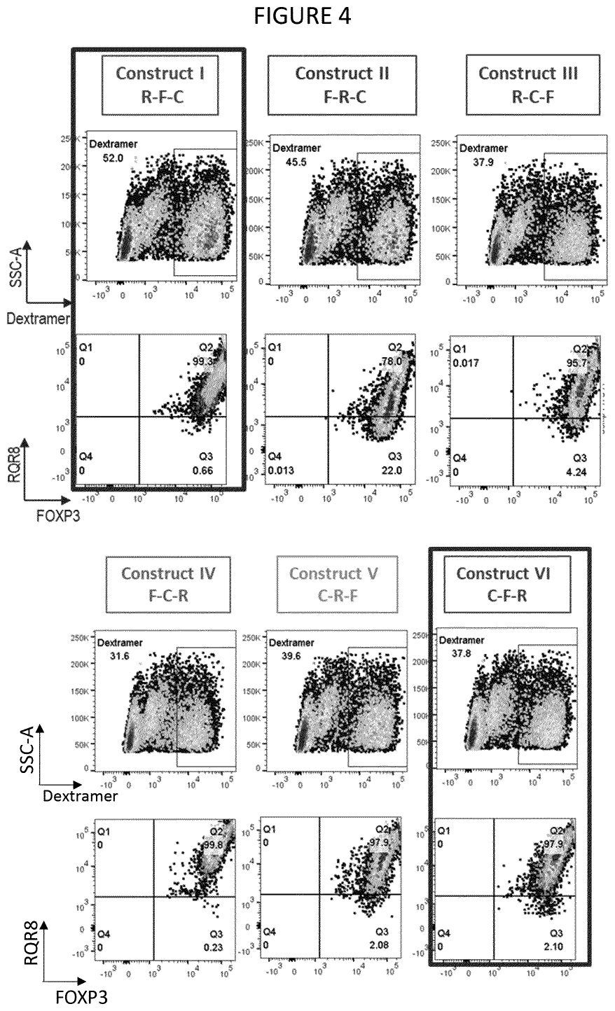

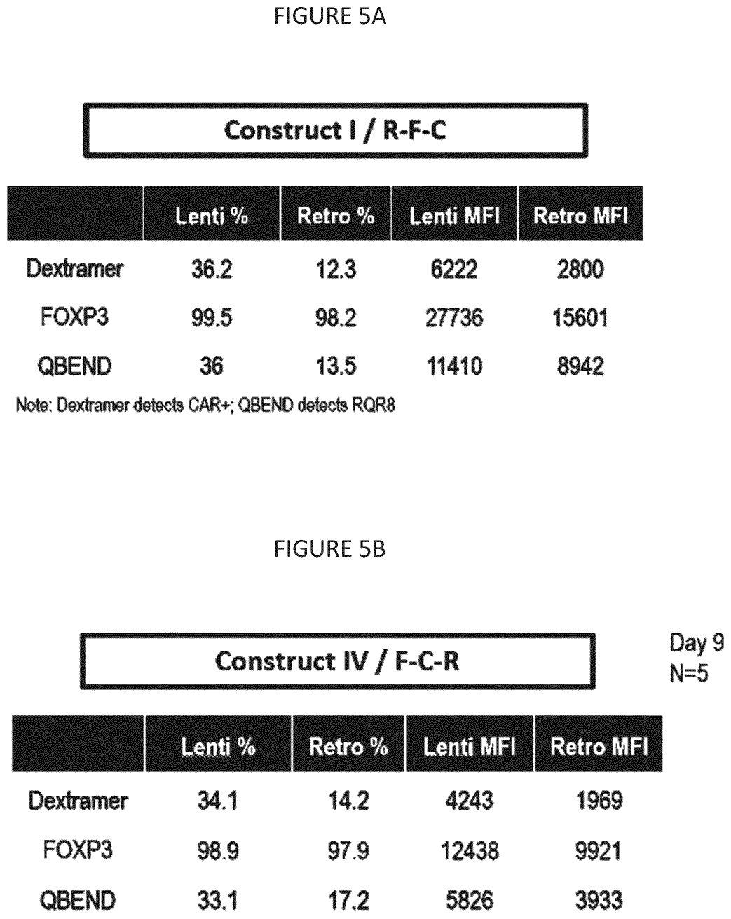

In the next step, transduction efficiency was investigated. FIG. 4 shows the expression results following transduction with a lentiviral vector. Good expression levels are shown, particularly for FOXP3 expression from construct I. Very high Dextramer can be seen for construct I as compared to the other experimental constructs. FIG. 5 sets out the comparison between γRV and lentiviral performance (showing a side-by-side comparison between the lentiviral and γRV vectors for constructs I (FIG. 5A) and IV (FIG. 5B). This shows that the lentiviral vector provides efficient cell transduction and gene expression. The results further show that the differential expression of the transduced components depends on the construct gene order, and is not dependent on the viral vector used. Particularly, the order of genes within construct I results in a higher expression of all three components as compared to construct IV.

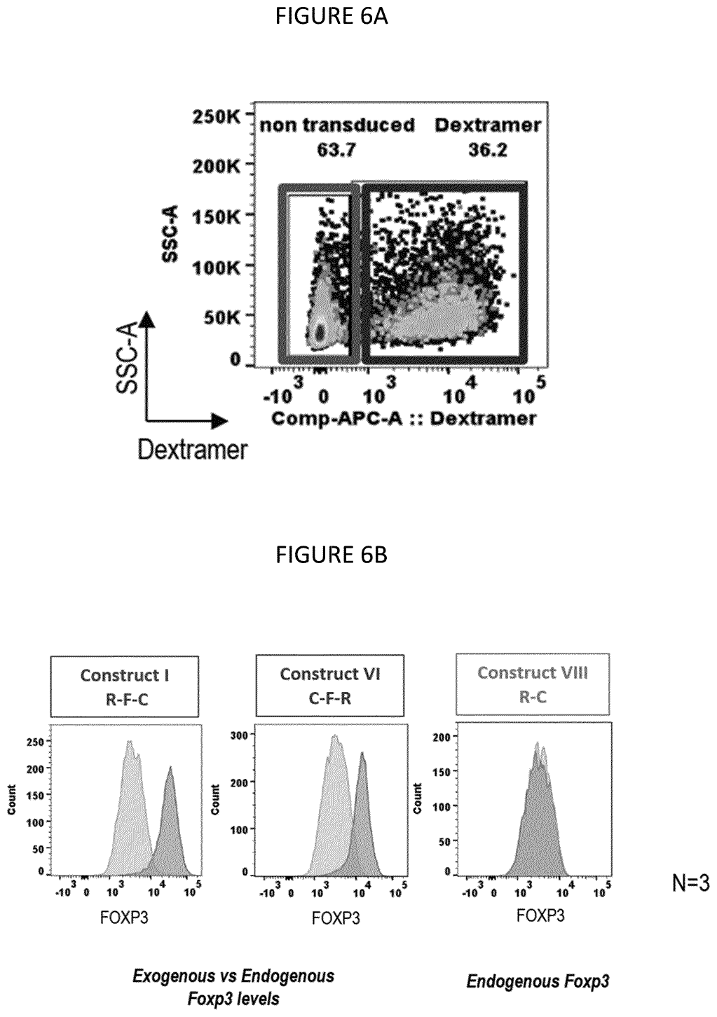

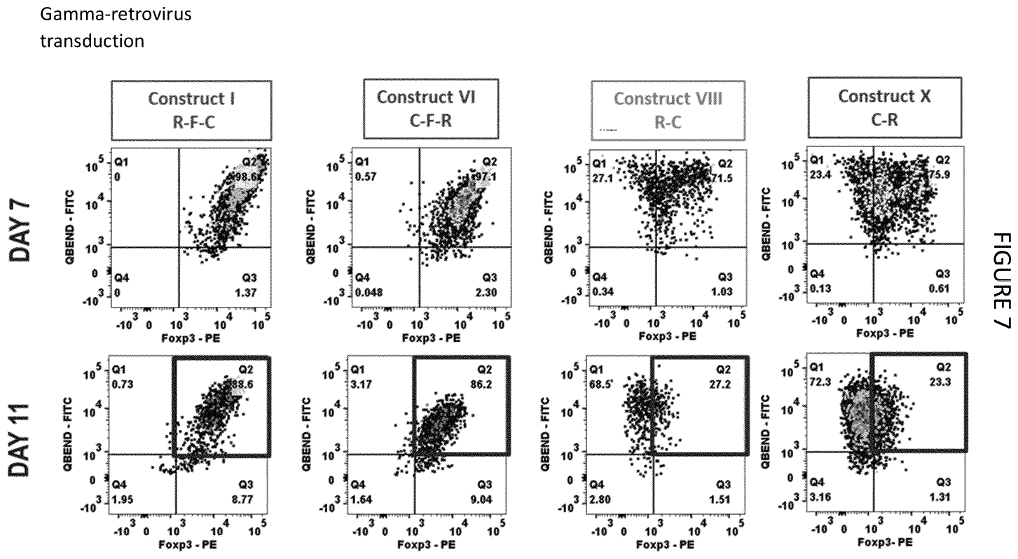

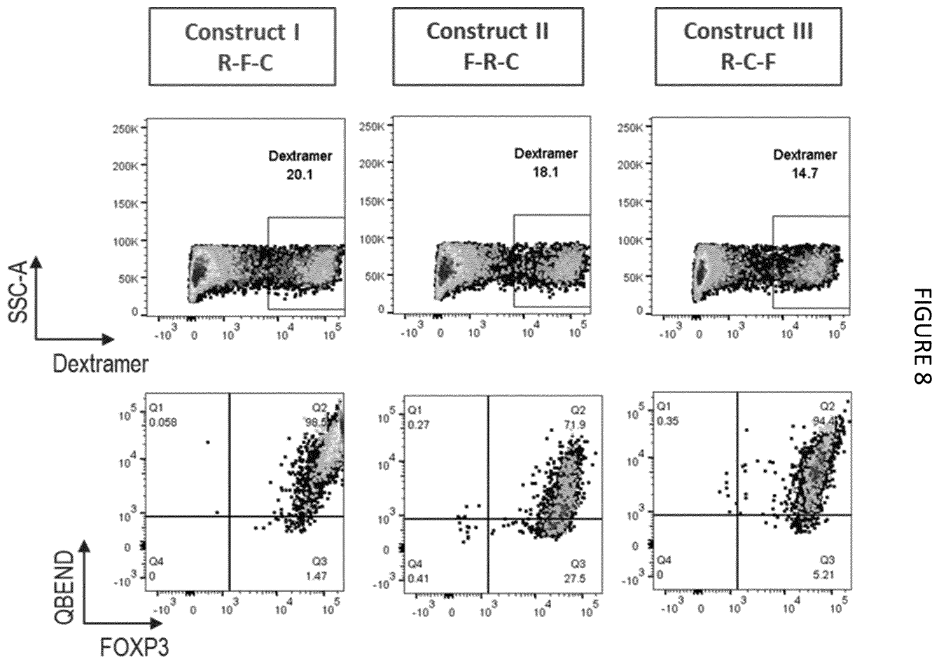

FIG. 6 shows that FOXP3-containing constructs enhance the level of FOXP3 protein in transduced cells, and this is particularly seen in FIG. 6B for construct I, which shows higher FOXP3 in transduced cells (exogenous and endogenous FOXP3), compared to non-transduced cells (which express only endogenous FOXP3), as demonstrated by the shift of the transduced peak to the right as compared to constructs VI and VIII (control construct VIII does not encode FOXP3).

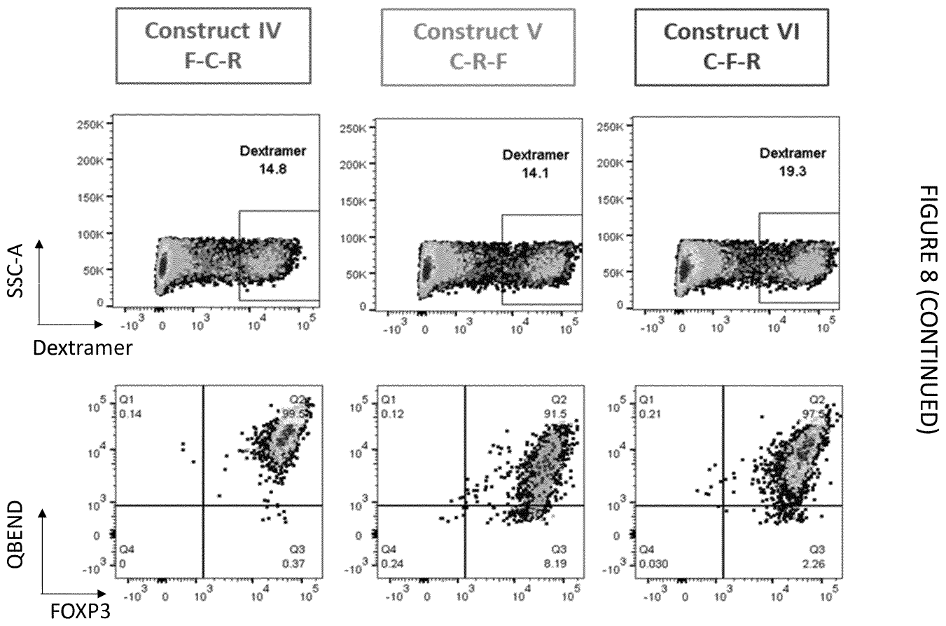

FIG. 7 shows exogenous FOXP3 expression in transduced Treg cells under lineage instability and that transduced FOXP3 confers stability in Tregs. Construct I in particular confers sustained FOXP3 expression independently of endogenous FOXP3 expression. Expression can still be seen at day 11.

FIG. 8 presents the results of further validation studies of the γRV expression constructs (Phoenix GP production/pMP71 backbone/RD114a envelope/LTR promoter). This again shows differential expression of the modules (encoded components) between constructs. Construct I was selected based on the improved expression levels and module convergence that can be seen. Levels of FOXP3 expression obtained with construct I are significantly higher than that seen for the other experimental constructs.

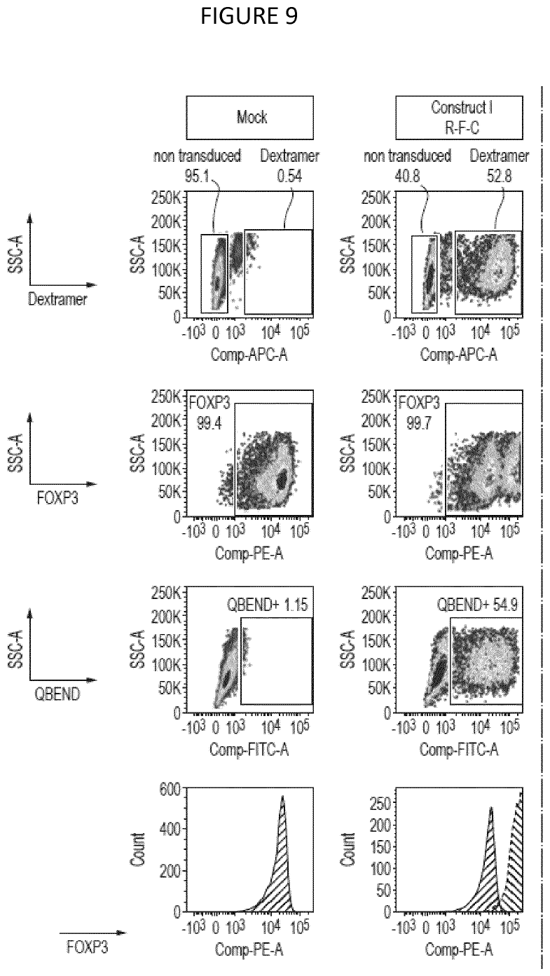

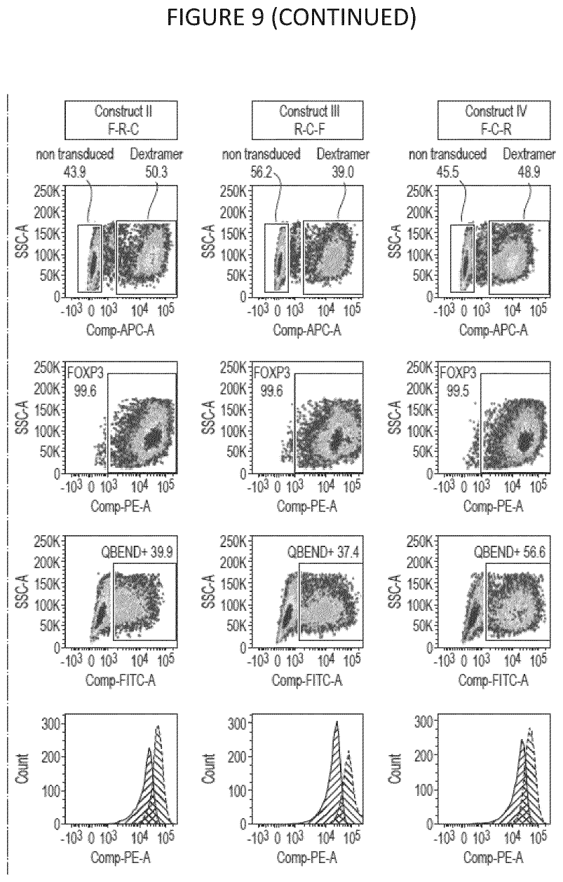

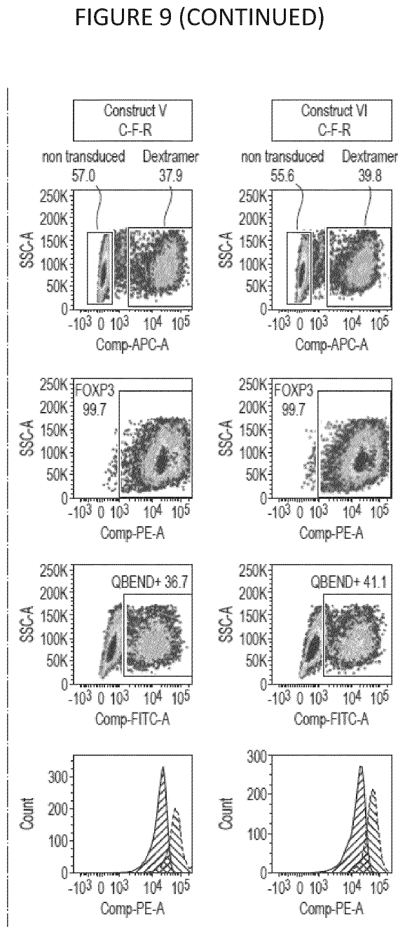

FIG. 9 confirms the expression patterns of the modules from the constructs using a lentiviral vector (HEK293T production/pLNT backbone/VSVg envelope/SFFV promoter). Again, differential expression is shown, and that the best expression is achieved with construct I.

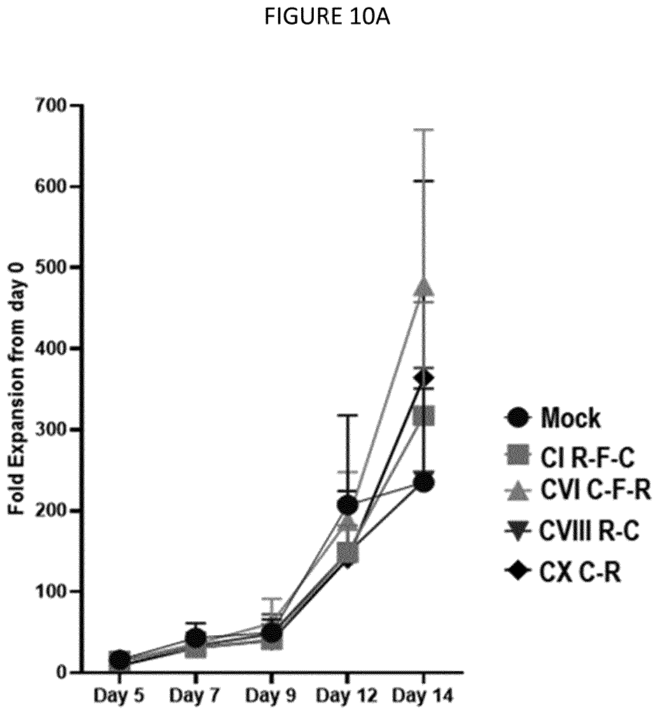

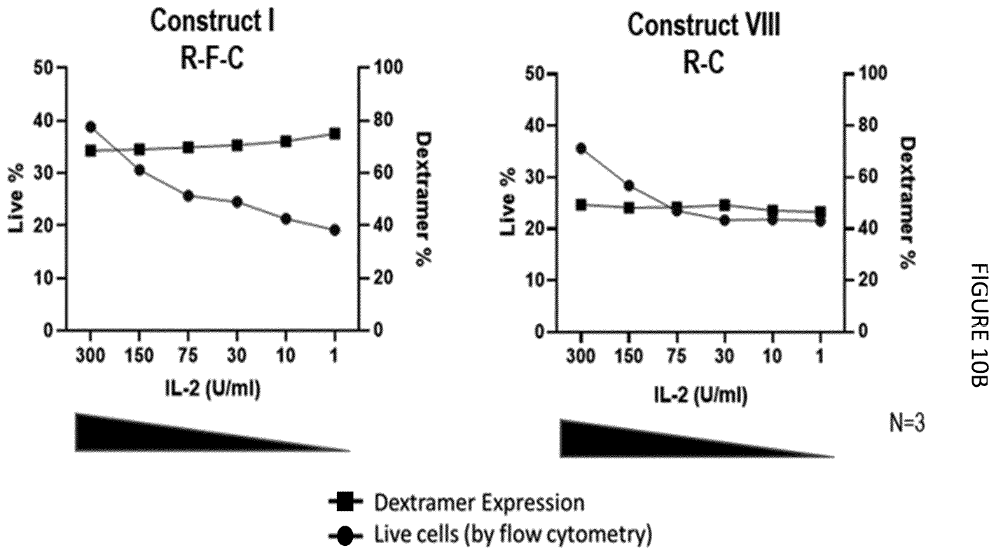

Possible effects on Treg homeostasis of the high FOXP3 expression levels conferred by construct I were investigated. FIG. 10 shows that FOXP3 expression does not seem to affect expansion and cell survival; cells expressing CAR and FOXP3 or CAR alone had similar overall expansion and survival upon IL-2 starvation. Thus the high FOXP3 expression levels achieved from construct I are not detrimental to the cells.

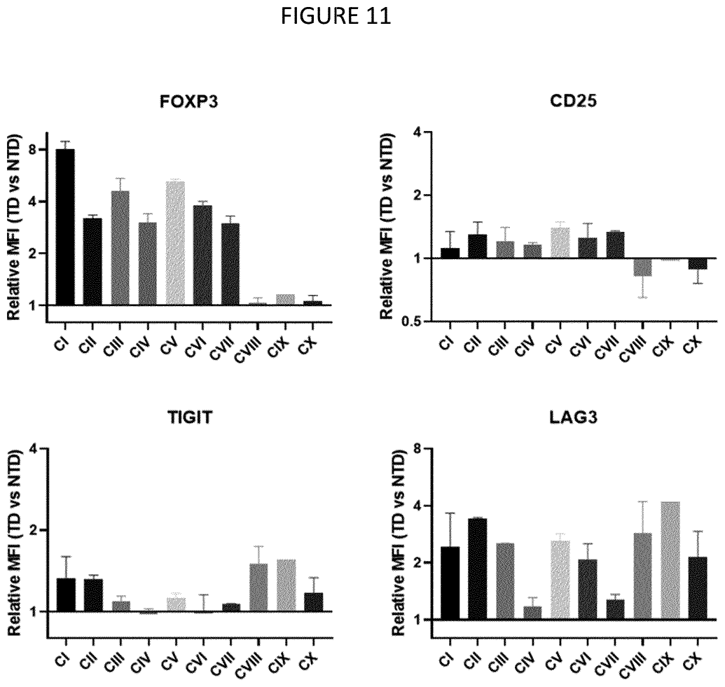

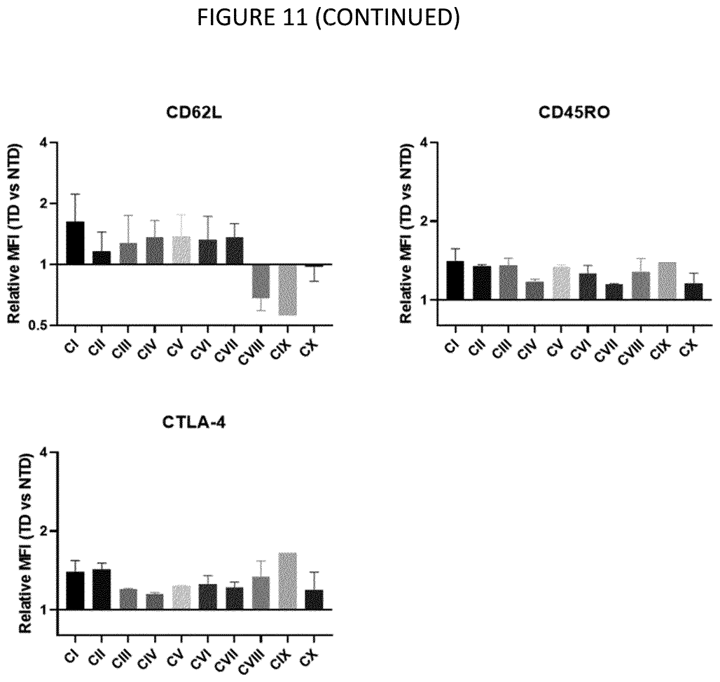

The expression of various markers (CD25, CD62L, TIGIT, LAG3, CTLA4 and CD45RO) was investigated to study the effects of FOXP3 expression on Treg phenotype. The results are shown FIG. 11. This shows that Treg cells transduced with construct I maintain Treg phenotypic lineage while demonstrating enhanced FOXP3 expression. There is no effect on Treg phenotype other than improved expression of FOXP3.

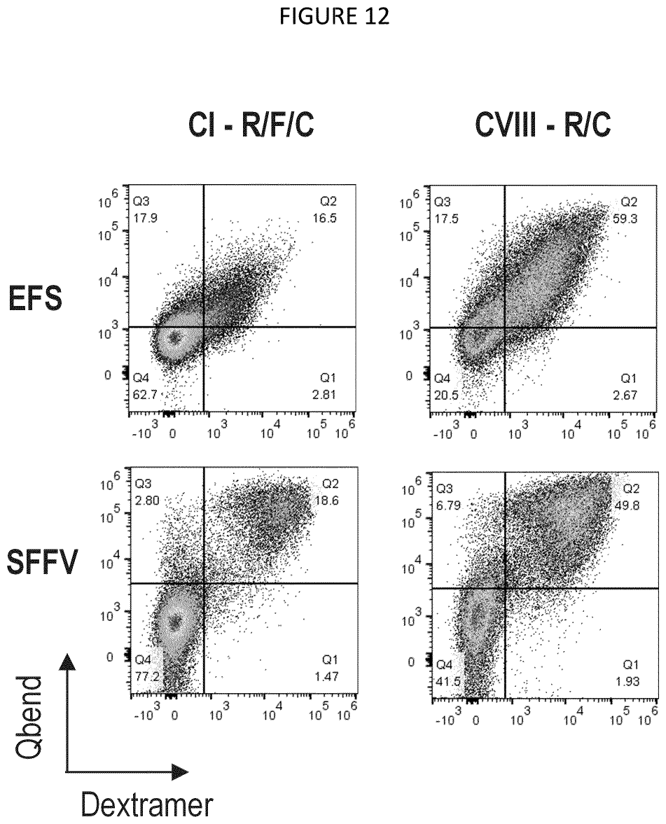

FIG. 12 shows the transduction efficiency seen when using different promoters together with construct I or construct VIII. The FACS plots show RQR8 expression (y-axis) and dextramer expression (x-axis) on CD4+ cells (Construct I is shown on the left and Construct VIII on the right). The data show that similar transduction efficiencies were obtained when using either the EFS or the SFFV promoter to express each constructs. When looking at transduction levels between constructs I and VIII, construct VIII demonstrated higher transduction, showing how transduction efficiency is affected by construct size (Construct VIII has 2 genes rather than three genes in Construct I).

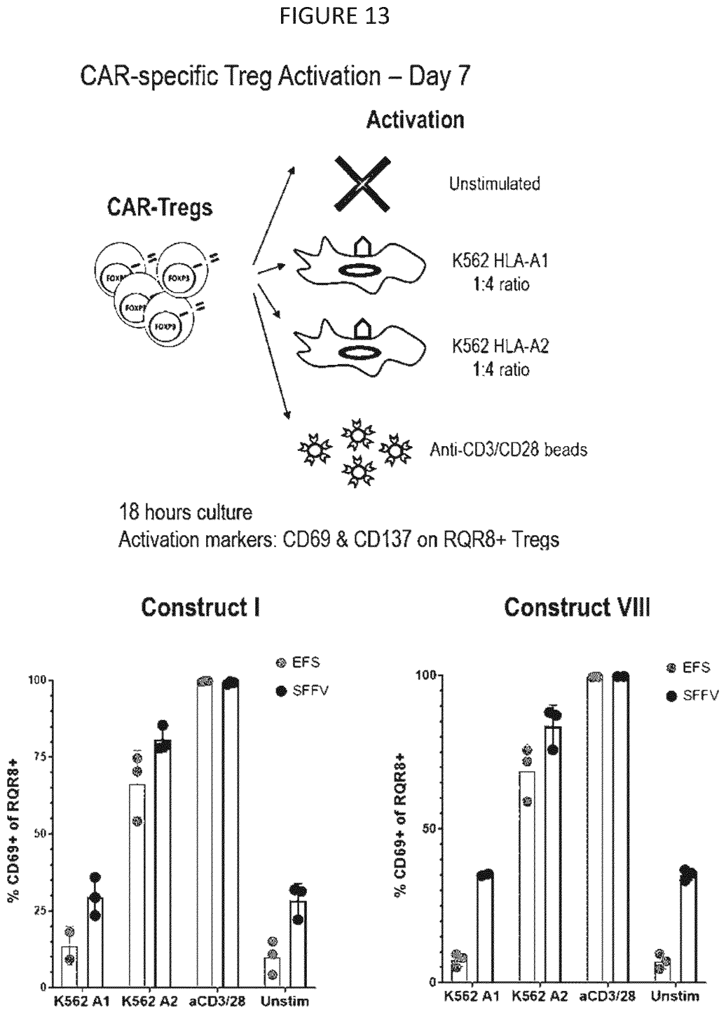

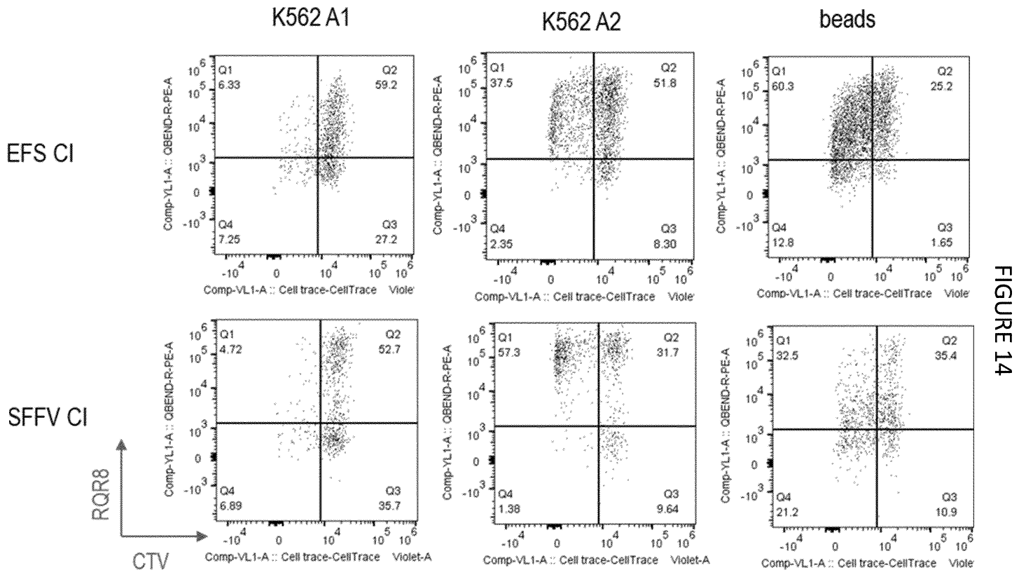

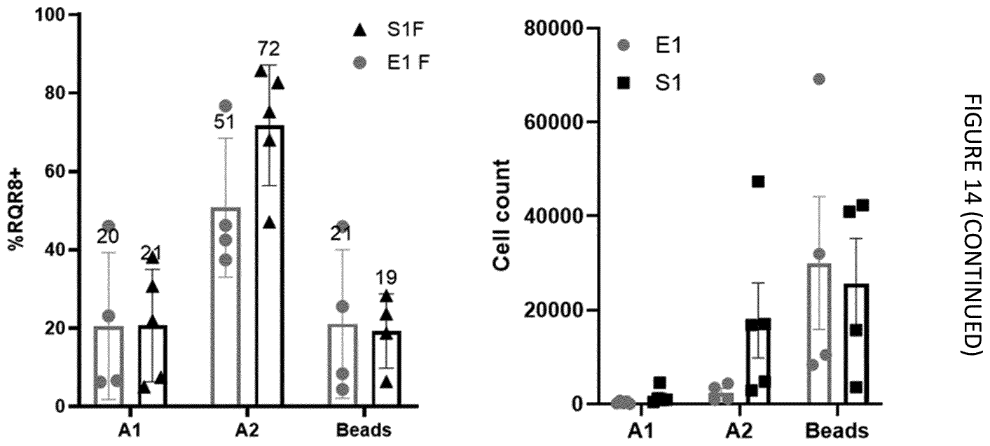

Activation of Tregs expressing the CAR from either Construct I or VIII was investigated when expression is controlled by the EFS or the SFFV promoter. FIG. 13 shows that Tregs transduced with either Construct I or VIII are activated through the CAR with HLA-A2 antigen, when either EFS or SFFV promoters are used. However, Treg activation is much higher when the SFFV promoter is used. A proliferation assessment of transduced Tregs (Construct I) was carried out and the results can be seen in FIG. 14. Representative FACS plots show co-staining of CTV and RQR8 for EFS-transduced (top panel) and SFFV-transduced cells (bottom panel). Graphs show frequency of RQR8+ cells and the total number of RQR8 cells after 5 days. Grey bars show EFS-transduced cells, black bars show SFFV-transduced cells. Dots represent individual donors and error bars show standard deviation (n=4-5). The data show that CAR-Tregs can proliferate after antigen specific stimulation and that use of the SFFV promoter enhances the proliferation.

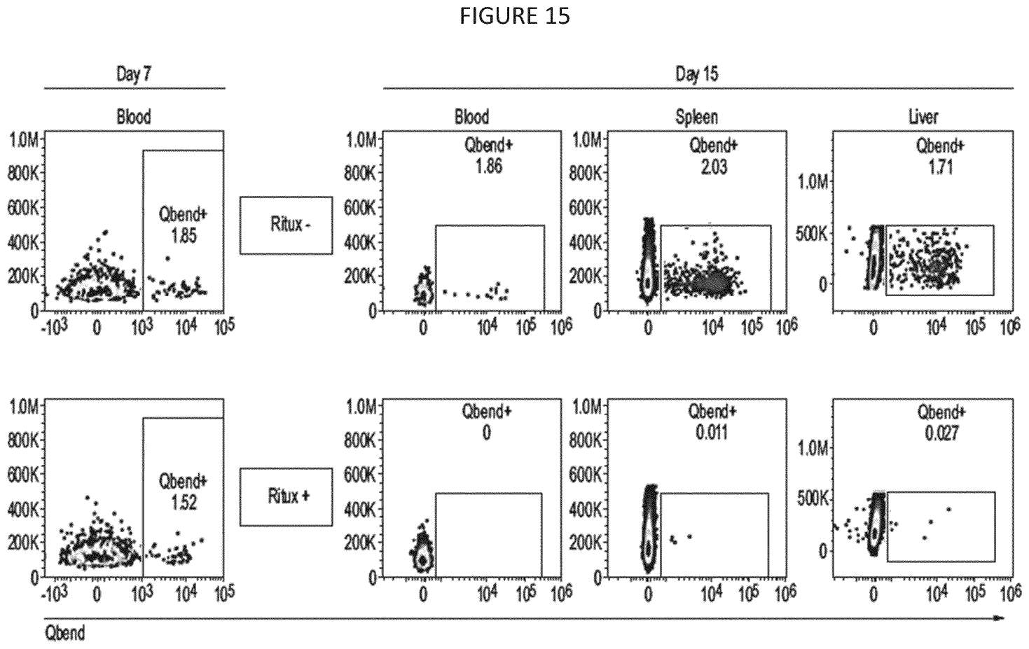

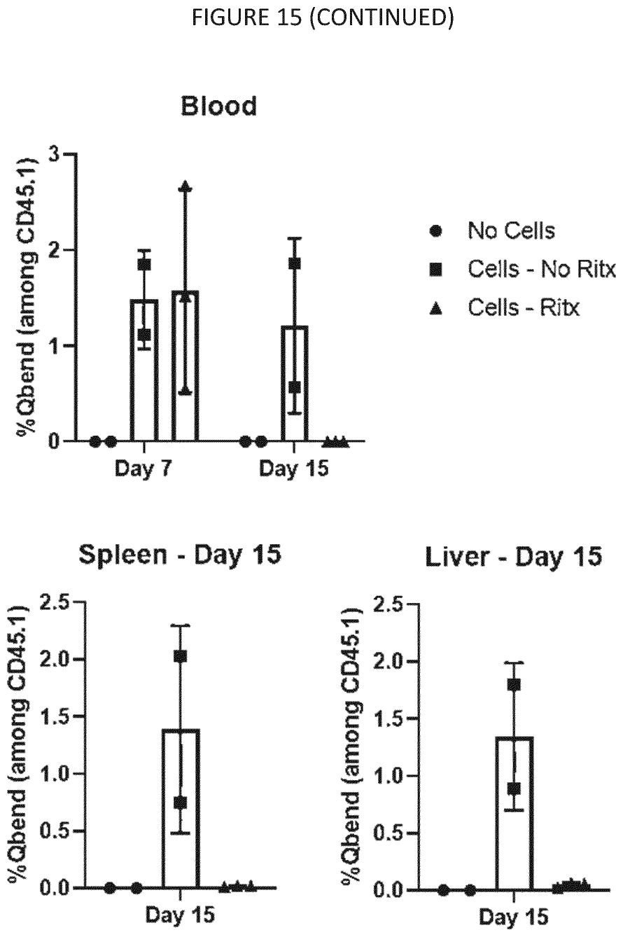

RQR8 is used within Construct I as a safety switch. To demonstrate its effectiveness in liver, murine T cells were transduced with a construct expressing RQR8 and were tracked in CD45.2 mice. FACS plots (FIG. 15) show staining with RQR8 antibody in the indicated tissues at day 7 and day 15 in the absence of aCD20 (top panel) and the presence of aCD20 (bottom panel). Graphs show cumulative data of the Qbend fraction within the CD45.1 gate at day 7 and day 15 for the blood and at day 15 for the spleen and lymph nodes. Dots indicate individual mice. Thus, it can be seen that cells can be detected in the liver at Day 15 in the absence of Rituximab, but that upon addition of Rituximab, cells are successfully deleted from the liver.

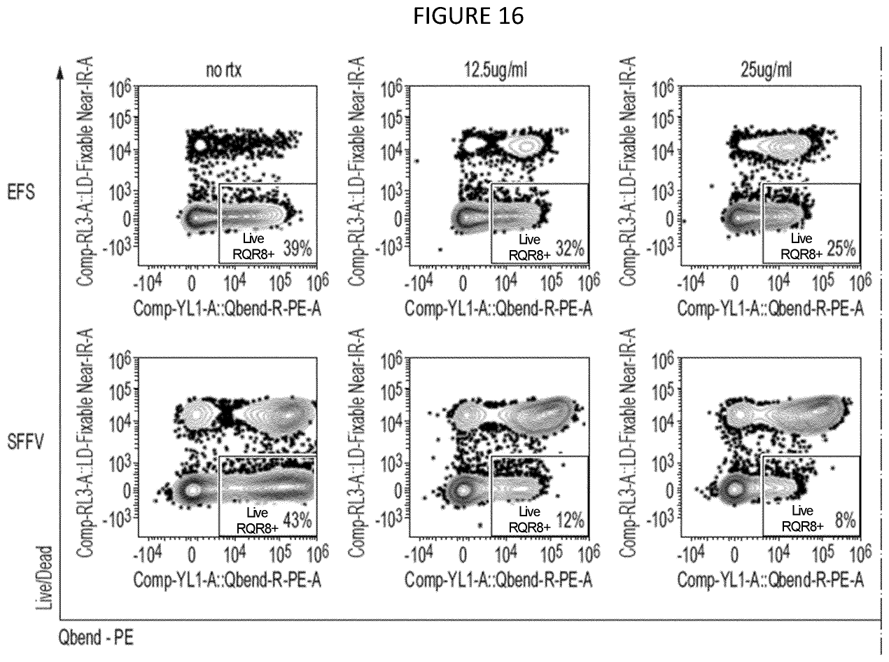

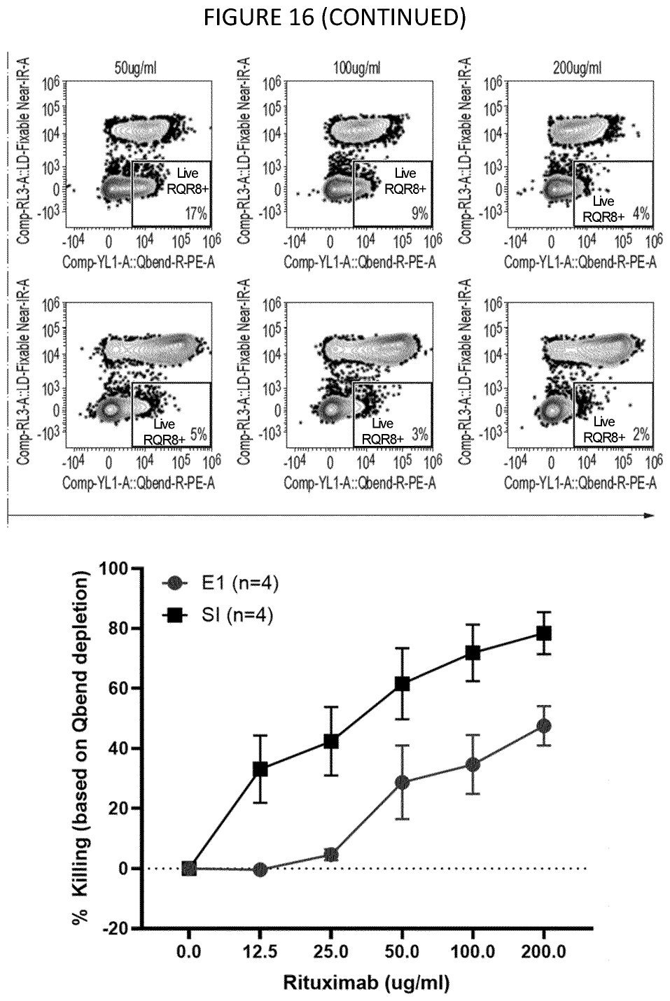

Further, an in vitro assay using cells transduced with Construct I with either EFS or SFFV promoters, shows that Rituximab can effectively deplete cells, but that increased sensitivity is provided when Construct I is expressed from an SFFV promoter (FIG. 16). FACS plots show percentage of live RQR8 (Qbend+) cells at indicated concentrations of Rituximab for EFS-transduced cells (top panel) and SFFV-transduced cells (bottom panel). Graphs show percentage killing calculated based on proportion of live RQR8+ cells in each condition relative to cells treated with complement alone. Graphs show the average of 4 individual experiments+/−standard deviation.

### CONCLUSION

In conclusion the results presented in FIGS. 2 to 16 confirm that improved expression of the desired components CAR (C), safety switch (R) and FOXP3 (F) may be obtained using construct I which encodes the components in the order R-F-C, as compared to constructs which encode the components in a different order, such as construct IV (F-C-R), or construct II (F-R-C), and with reduced incidence of undesired expression patterns, where for example, the expression of one of the components is significantly reduced or absent. Unusually and surprisingly high levels of FOXP3 expression are achieved, and these are shown not to be detrimental to the Treg expansion and survival.

Further, the data show that there are clear additional advantages to using a SFFV promoter for construct expression, including an unexpectedly improved activation of the CAR in the presence of antigen, increased proliferation of transduced Tregs and increased sensitivity of the transduced cells to Rituximab mediated depletion.

1. An expression construct comprising a nucleic acid molecule comprising 5′ to 3′:
(i) a first nucleotide sequence encoding a safety switch polypeptide comprising a suicide moiety;
(ii) a second nucleotide sequence encoding FOXP3; and
(iii) a third nucleotide sequence encoding a chimeric antigen receptor (CAR);
wherein said first, second, and third nucleotide sequences are operably linked to an SFFV promoter.

2. The expression construct of claim 1, wherein:
(a) the safety switch polypeptide, FOXP3 and the CAR are expressible from the nucleic acid molecule as separate polypeptide entities;
(b) said first, second, and third nucleotide sequences are separated by nucleotide sequences encoding self-cleavage sequences;
(c) the nucleic acid molecule does not comprise any other coding nucleotide sequence;
(d) the safety switch polypeptide comprises a suicide moiety which is recognised by an antibody, and wherein binding of the antibody to the safety switch polypeptide, when expressed on the surface of a cell, causes the cell to be eliminated, optionally wherein the suicide moiety is a CD20 epitope which is recognised by the antibody Rituximab;
(e) the safety switch polypeptide comprises a sequence having the formula:
R1-L-R2-St
wherein R1 and R2 are Rituximab-binding epitopes;
St is a stalk sequence which, when the polypeptide is expressed at the surface of a cell, causes the R1 and R2 epitopes to be projected from the cell surface; and
L is a linker sequence
(f) the safety switch polypeptide of part (e), wherein the safety switch polypeptide is:
(i) the polypeptide RQR8 having the sequence of SEQ ID NO. 1, or a sequence with at least 80% sequence identity thereto; or
(ii) a polypeptide having the sequence of SEQ ID NO. 92 or 93, or a sequence with at least 80% sequence identity to SEQ ID NO. 92 or 93; and/or
(g) said first, second and third nucleotide sequences are separated by nucleotide sequences encoding self-cleavage sequences which are 2A sequences, optionally wherein the self-cleavage sequences are 2A sequences, optionally wherein the self-cleavage sequence between the safety switch polypeptide and FOXP3 is a P2A sequence and the self-cleavage sequence between FOXP3 and the CAR is a T2A sequence.

3-8. (canceled)

9. The expression construct of claim 1, wherein:
(a) the FOXP3 is a polypeptide comprising an amino acid sequence having at least 70% sequence identity to SEQ ID NO. 2 or 7, or a polypeptide comprising an amino acid sequence which comprises a deletion of amino acids 72-106 or 246-272 relative to SEQ ID NO. 2 or 7, preferably wherein the FOXP3 is a polypeptide comprising or consisting of SEQ ID NO. 2;
(b) (i) the CAR is directed against an HLA molecule, optionally wherein the HLA molecule is HLA-A2; or
(ii) the CAR is not directed against MHC class II;
(c) (i) the CAR comprises an antigen binding domain which comprises VH CDR1, 2 and 3 sequences as set forth in SEQ ID NOs. 11, 12 and 13 respectively and VL CDR1, 2 and 3 sequences as set forth in SEQ ID NOs. 14, 15, 16 respectively; or
(ii) the CAR comprises an antigen binding domain which comprises VH CDR1, 2 and 3 sequences as set forth in SEQ ID NOs. 20, 21 and 22 respectively and VL CDR1, 2 and 3 sequences as set forth in SEQ ID NOs. 23, 24, 25 respectively;
wherein one or more of said CDR sequences of (i) or (ii) may optionally comprise 1 to 3 amino acid modifications relative to an aforementioned CDR sequence, particularly wherein one or more of said CDR sequences may optionally be modified by substitution, addition or deletion of 1 to 3 amino acids;
(d) (i) the CAR comprises an antigen binding domain which comprises a VH domain comprising the sequence as set forth in SEQ ID NO. 17, or a sequence having at least 70% sequence identity thereto, and a VL domain comprising the sequence as set forth in SEQ ID NO. 18, or a sequence having at least 70% sequence identity thereto; or
(ii) the CAR comprises an antigen binding domain which comprises a VH domain comprising the sequence as set forth in SEQ ID NO. 26, or a sequence having at least 80% sequence identity thereto, and a VL domain comprising the sequence as set forth in SEQ ID NO. 27, or a sequence having at least 70% sequence identity thereto;
(e) the CAR comprises an antigen binding domain in the form of a scFv;
(f) the CAR comprises an antigen binding domain which comprises:
(i) the sequence as set forth in SEQ ID NO. 19 or 88 or a sequence having at least 80% sequence identity thereto; or
(ii) the sequence as set forth in SEQ ID NO. 28 or a sequence having at least 80% sequence identity thereto;
(g) the CAR comprises:
(i) a hinge domain selected from the hinge regions of CD8, CD28, CD4, CD7, or an immunoglobulin, or a part or variant thereof;
(ii) a transmembrane domain selected from the transmembrane domains of CD8α, CD28, CD4, CD3ζ CD45, CD9, CD16, CD22, CD33, CD64, CD80, CD86, CD134 (OX40), CD137 (4-1BB), or CD154, or a part thereof or variant thereof;
(iii) optionally a co-stimulatory domain selected from the intracellular domains of CD28, OX40, 41BB, ICOS or TNFRSF25 and
(iv) an intracellular signaling domain selected from the endodomains of the ζ chain of the T-cell receptor or any of its homologs, CD3 polypeptide endodomains, syk family tyrosine kinases), src family tyrosine kinases CD2, CD5 and CD28, FcγRIII, FcsRI, cytoplasmic tails of Fc receptors, immunoreceptor tyrosine-based activation motif (ITAM) bearing cytoplasmic receptors or combinations thereof; and/or
(h) (i) the CAR comprises a human CD8 hinge domain or a variant thereof and a human CD8 transmembrane domain; and/or
(ii) the CAR comprises an endodomain comprising a human CD28 co-stimulatory domain and a human CD3ζ signaling domain; and/or
(iii) the CAR comprises an endodomain comprising a STAT5 association motif, a JAK1 and/or JAK 2 binding motif and optionally a JAK 3 binding motif, preferably wherein the endodomain of the CAR comprises one or more sequences from an endodomain of an interleukin receptor (IL) receptor.

10-17. (canceled)

18. The expression construct of claim 1, wherein the nucleic acid molecule comprises 5′ to 3′:
(i) a first nucleotide sequence encoding a safety switch polypeptide comprising the sequence of SEQ ID NO. 10, 94, or 95, or a sequence with at least 80% sequence identity thereto;
(ii) a nucleotide sequence encoding a P2A cleavage sequence;
(iii) a second nucleotide sequence encoding a FOXP3 polypeptide comprising the sequence of SEQ ID NO. 2, or a sequence with at least 70% sequence identity thereto;
(iv) a nucleotide sequence encoding a T2A cleavage sequence; and
(vi) a third nucleotide sequence encoding a CAR directed against HLA-A2, wherein the CAR comprises:
(a) a leader sequence comprising or consisting of a sequence as set out in SEQ ID NO. 66, or a sequence having at least 80% sequence identity thereto;
(b) an antigen binding domain comprising or consisting of a sequence as set out in SEQ ID NO. 19 or 88, or a sequence having at least 80% sequence identity thereto;
(c) a CD8α hinge and transmembrane domain sequence comprising or consisting of the sequence as set forth in SEQ ID NO. 68, or a sequence having at least 80% sequence identity thereto;
(d) a CD28 co-stimulatory domain comprising or consisting of the sequence as set forth in SEQ ID NO. 71, or a sequence having at least 80% sequence identity thereto;
(c) a CD3ζ signalling domain comprising or consisting of the sequence as set forth in SEQ ID NO. 72, or a sequence having at least 80% sequence identity thereto.

19-20. (canceled)

21. A vector comprising the expression construct of claim 1 and, optionally
wherein the vector is a viral vector, further optionally a lentiviral vector or gamma-retroviral vector.

22. (canceled)

23. A cell comprising:
(a) the expression construct of claim 1; or
(b) a vector comprising said expression construct, and optionally wherein the vector is a viral vector, further optionally a lentiviral vector or gamma-retroviral vector, optionally
wherein the cell is a production host cell.

24. The cell of claim 23, wherein:
(a) the cell co-expresses the safety switch polypeptide, and the CAR at the cell surface;
(b) the cell is:
(i) an immune cell or a progenitor or precursor therefor, preferably a T cell or a precursor therefor, more preferably a Treg or Tcon cell or a precursor therefor; or
(ii) a stem cell, preferably an iPSC cell; and/or
(c) the cell is a Treg cell.

25-26. (canceled)

27. A cell population comprising a cell of claim 23.

28. A pharmaceutical composition comprising:
(a) a cell comprising:
(i) the expression construct of claim 1; or
(ii) a vector comprising said expression construct, and optionally wherein the vector is a viral vector, further optionally a lentiviral vector or gamma-retroviral vector, optionally
wherein the cell is a production host cell;
(b) a cell population comprising said cell; or
(c) said vector.

29. A method of treating or preventing a disease which comprises the step of administering to a subject:
(a) a cell comprising:
(i) the expression construct of claim 1; or
(ii) a vector comprising said expression construct, and optionally wherein the vector is a viral vector, further optionally a lentiviral vector or gamma-retroviral vector, optionally wherein the cell is a production host cell;
(b) a cell population comprising said cell; or
(c) a pharmaceutical composition comprising:
(i) said cell;
(ii) a cell population comprising said cell; or
(iii) said vector.

30. The method according to claim 29, wherein:
(a) the disease is treated by adoptive cell transfer therapy; or
(b) the disease is an infectious, neurodegenerative or inflammatory disease,
(c) the disease is treated by inducing immunosuppression.

31-32. (canceled)

33. A method of inducing tolerance to a transplant; treating and/or preventing graft-versus-host disease (GvHD), an autoimmune or allergic disease; or to promote tissue repair and/or tissue regeneration; or to ameliorate inflammation which comprises the step of administering to a subject:
(a) a cell, particularly a Treg cell, comprising:
(i) the expression construct of claim 1; or
(ii) a vector comprising said expression construct, and optionally wherein the vector is a viral vector, further optionally a lentiviral vector or gamma-retroviral vector, optionally
wherein the cell is a production host cell;
(b) a cell population comprising said cell, particularly comprising a Treg cell; or
(c) a pharmaceutical composition comprising:
(i) said cell;
(ii) a cell population comprising said cell; or
(iii) said vector,
wherein the pharmaceutical composition particularly comprises a Treg cell.

34. A method according to claim 33 which comprises the following steps:
(i) isolation or provision of a Treg-enriched cell sample optionally from a subject;
(ii) introduction into the Treg cells of said expression construct or said vector; and
(iii) administering the Treg cells from (ii) to the subject.

35. (canceled)

36. A method according to claim 33, wherein:
(a) the subject is a transplant recipient undergoing immunosuppression therapy;
(b) the subject is a transplant recipient undergoing immunosuppression therapy and the transplant is selected from a liver, kidney, heart, lung, pancreas, intestine, stomach, bone marrow, vascularized composite tissue graft, and skin transplant;
(c) the subject is a liver transplant recipient undergoing immunosuppression therapy and the CAR comprises an antigen binding domain which is capable of specifically binding to an antigen selected from: a HLA antigen present in the transplanted liver but not in the recipient, or a liver-specific antigen such as NTCP;
(d) the CAR comprises an antigen binding domain which is capable of specifically binding to an antigen whose expression is up-regulated during rejection or tissue inflammation such as CCL19, MMP9, SLC1A3, MMP7, HMMR, TOP2A, GPNMB, PLA2G7, CXCL9, FABP5, GBP2, CD74, CXCL10, UBD, CD27, CD48, CXCL11;
(e) the CAR comprises an antigen binding domain which is capable of specifically binding to a HLA antigen that is present in the graft donor but not in the graft recipient;
(f) the CAR comprises an antigen binding domain which is capable of specifically binding to the HLA antigen HLA-A2 that is present in the graft donor but not in the graft recipient; and/or
(g) the CAR comprises an antigen binding domain comprises a sequence as set forth in SEQ ID NO. 19, 88 or 28, or a sequence with at least 80% identity to SEQ ID NO. 19, 88 or 28.

37. (canceled)

38. The method according to claim 32, wherein the autoimmune or allergic disease is selected from inflammatory skin diseases including psoriasis and dermatitis; responses associated with inflammatory bowel disease, including Crohn's disease and ulcerative colitis; dermatitis; allergic conditions including food allergy, eczema and asthma; rheumatoid arthritis; systemic lupus erythematosus (SLE) including lupus nephritis and cutaneous lupus; diabetes mellitus including type 1 diabetes mellitus or insulin dependent diabetes mellitus; CIPD, multiple sclerosis, neurodegenerative diseases including. ALS; and juvenile onset diabetes.

39. A method for making a cell, wherein the cell comprises;
(a) the expression construct of claim 1; or
(b) a vector comprising said expression construct, and optionally wherein the vector is a viral vector, further optionally a lentiviral vector or gamma-retroviral vector, and
wherein the method comprises the step of introducing into the cell:
(i) said expression construct; or
(ii) said vector, optionally
wherein the cell is a Treg cell and the method comprises isolating or providing a cell-containing sample comprising Tregs, and/or Tregs are enriched and/or generated from the cell-containing sample prior to or after the step of introducing the expression construct or vector in the cells.

40. (canceled)

41. A method of enhancing the expression of FOXP3 from a nucleic acid molecule encoding a chimeric antigen receptor (CAR), a safety switch and FOXP3 in a cell, comprising selecting a nucleic acid molecule as defined in claim 1, preparing an expression construct comprising said nucleic acid molecule, and introducing said expression construct into said cell, optionally wherein expression of the CAR from said nucleic acid molecule is also enhanced in said cell.

42-44. (canceled)

45. The method of claim 41, wherein the method comprises producing a nucleic acid molecule comprising said first, second and third nucleotide sequences in the specified order and preparing an expression construct comprising said nucleic acid molecule comprising said first, second, and third nucleotide sequences operably linked to a SFFV promoter.

46. A method of increasing the sensitivity of a cell expressing a safety switch polypeptide comprising a suicide moiety of at least one CD20 epitope recognised by Rituximab, to Rituximab, comprising introducing into said cell a nucleic acid molecule comprising a nucleotide sequence encoding said safety switch polypeptide wherein said nucleotide sequence is operably linked to a SFFV promoter.

47. (canceled)
